(19) United States
(12) Patent Application Publication
(10) Pub. No.: US 2024/0382488 A1
(43) Pub. Date: Nov. 21, 2024

(54) NEK7 INHIBITORS

(71) Applicant: Halia Therapeutics, Inc., Lehi, UT (US)

(72) Inventors: David James BEARSS, Lehi, UT (US); John Sai Keong KAUWE, III, Lehi, UT (US); Alexis Henri Abel MOLLARD, Lehi, UT (US)

(21) Appl. No.: 18/553,128

(22) PCT Filed: Mar. 29, 2022
(86) PCT No.: PCT/US2022/022283

§ 371 Date: Sep. 28, 2023

Related U.S. Application Data
(60) Provisional application No. 63/185,274, filed on May 6, 2021.
(60) Provisional application No. 63/167,523, filed on Mar. 29, 2021.

(51) Int. Cl. A61K 31/519; A61K 31/437; C07D 471/04; C07D 487/04

(52) U.S. Cl. CPC A61K 31/519; A61K 31/437; C07D 471/04; C07D 487/04

(57) ABSTRACT

Compounds having activity as inhibitors of NEK7 are provided. The compounds have Structure (I): or a pharmaceutically acceptable salt, stereoisomer or prodrug thereof, wherein A, X, Y, R¹, R², R³, R⁴, R⁵, R⁶, R⁷, and R⁸ are as defined herein. Methods associated with preparation and use of such compounds, pharmaceutical compositions comprising such compounds and methods to treat diseases or disorders (e.g., cancer) are also provided.

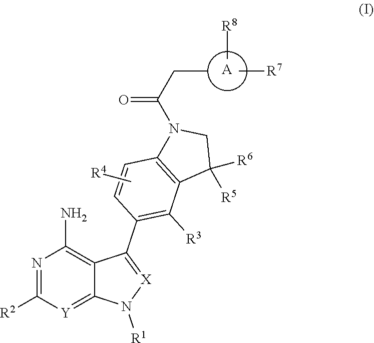

### BACKGROUND

### Technical Field

Embodiments of the present disclosure are generally directed to compounds and methods for their preparation and use as therapeutic or prophylactic agents, for example for treatment of disease (e.g., cancer).

### Description of the Related Art

NEK7 is a member of the family of NIMA-related kinases (NEKs). NEK7 is a serine/threonine kinase essential for mitotic entry, cell cycle progression, cell division, and mitotic progression. It is expressed in a variety of tissues such as the brain, heart, lung, liver, and spleen. Overexpression of NEK7 induces the production of abnormal cells, which has an intimate connection to tumors, such as retinoblastoma, gallbladder cancer and carcinoma of the head and neck.

Accordingly, there is a need to develop inhibitors that will directly target NEK7 as potential treatments for known diseases (e.g., cancer). Embodiments of the present disclosure fulfill this need and provide further related advantages.

### BRIEF SUMMARY

In brief, embodiments of the present disclosure provide compounds, including pharmaceutically acceptable salts, stereoisomers, and prodrugs thereof, which are useful as treatments for diseases (e.g., cancer).

One embodiment provides compounds of Structure (I):

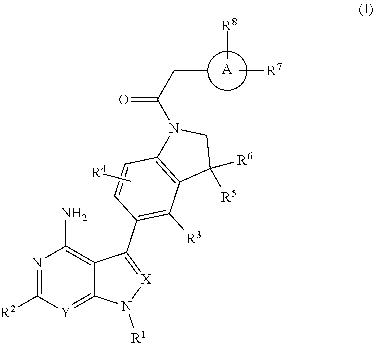

or pharmaceutically acceptable salts, stereoisomers or prodrug thereof, wherein each of A, X, Y, R¹, R², R³, R⁴, R⁵, R⁶, R⁷, and R⁸ are as defined herein.

In another embodiment, pharmaceutical compositions comprising the disclosed compounds, and methods of use of the same for treatment of inflammation are also provided.

### DETAILED DESCRIPTION

In the following description, certain specific details are set forth in order to provide a thorough understanding of various embodiments of the disclosure. However, one skilled in the art will understand that the disclosure may be practiced without these details.

Unless the context requires otherwise, throughout the present specification and claims, the word “comprise” and variations thereof, such as, “comprises” and “comprising” are to be construed in an open, inclusive sense, that is, as “including, but not limited to”.

In the present description, any concentration range, percentage range, ratio range, or integer range is to be understood to include the value of any integer within the recited range and, when appropriate, fractions thereof (such as one tenth and one hundredth of an integer), unless otherwise indicated. As used herein, the terms “about” and “approximately” mean±20%, ±10%, ±5%, or ±1% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination thereof of the alternatives.

Reference throughout this specification to “one embodiment” or “an embodiment” means that a particular feature, structure or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

Unless defined otherwise, all technical and scientific terms used herein have the same meaning as is commonly understood by one of skill in the art to which this disclosure belongs. As used in the specification and claims, the singular form “a”, “an” and “the” include plural references unless the context clearly dictates otherwise.

“Amino” refers to the —NH₂ radical.

“Carboxy” or “carboxyl” refers to the —CO₂H radical.

“Cyano” refers to the —CN radical.

“Hydroxy” or “hydroxyl” refers to the —OH radical.

“Nitro” refers to the −NO₂ radical.

“Oxo” refers to the ═0 substituent.

“Thiol” refers to the —SH substituent.

“Thioxo” refers to the ═S substituent.

“Alkyl” refers to a saturated, straight or branched hydrocarbon chain radical consisting solely of carbon and hydrogen atoms, having, for example, from one to twelve carbon atoms (C₁-C₁₂ alkyl), one to eight carbon atoms (C₁-C₈ alkyl) or one to six carbon atoms (C₁-C₆ alkyl), or any value within these ranges, such as C₄-C₆ alkyl and the like, and which is attached to the rest of the molecule by a single bond, e.g., methyl, ethyl, n-propyl, 1-methylethyl (iso-propyl), n-butyl, n-pentyl, 1,1-dimethylethyl (t-butyl), 3-methylhexyl, 2-methylhexyl and the like. The number of carbons referred to relates to the carbon backbone and carbon branching, but does not include carbon atoms belonging to any substituents. Unless stated otherwise specifically in the specification, an alkyl group is optionally substituted.

“Alkenyl” refers to an unsaturated, straight or branched hydrocarbon chain radical consisting solely of carbon and hydrogen atoms, which contains one or more carbon-carbon double bonds, having from two to twelve carbon atoms (C₂-C₁₂ alkenyl), two to eight carbon atoms (C₂-C₅ alkenyl) or two to six carbon atoms (C₂-C₆ alkenyl), or any value within these ranges, and which is attached to the rest of the molecule by a single bond, e.g., ethenyl, prop-1-enyl, but-1-enyl, pent-1-enyl, penta-1,4-dienyl, and the like. The number of carbons referred to relates to the carbon backbone and carbon branching, but does not include carbon atoms belonging to any substituents. Unless stated otherwise specifically in the specification, an alkenyl group is optionally substituted.

The term “alkynyl” refers to unsaturated straight or branched hydrocarbon radical, having 2 to 12 carbon atoms (C₂-C₁₂ alkynyl), two to nine carbon atoms (C₂-C₉ alkynyl), or two to six carbon atoms (C₂-C₆ alkynyl), or any value within these ranges, and having at least one carbon-carbon triple bond. Examples of alkynyl groups may be selected from the group consisting of ethynyl, propargyl, but-1-ynyl, but-2-ynyl and the like. The number of carbons referred to relates to the carbon backbone and carbon branching, but does not include carbon atoms belonging to any substituents. Unless stated otherwise specifically in the specification, an alkynyl group is optionally substituted.

“Alkoxy” refers to a radical of the formula —ORₐ where Rₐ is an alkyl radical as defined above containing one to twelve carbon atoms (C₁-C₁₂ alkoxy), one to eight carbon atoms (C₁-C₈ alkoxy) or one to six carbon atoms (C₁-C₆ alkoxy), or any value within these ranges. Unless stated otherwise specifically in the specification, an alkoxy group is optionally substituted.

“Aminyl” refers to a radical of the formula —NRₐR_{b}, where Rₐ and R_{b} are each independently H or C₁-C₆ alkyl as defined above. When both of Rₐ and R_{b} are H, an “aminyl” group is the same as an “amino” group as defined above. The C₁-C₆ alkyl portion of an aminyl group is optionally substituted unless stated otherwise.

“Aromatic ring” refers to a cyclic planar molecule or portion of a molecule (i.e., a radical) with a ring of resonance bonds that exhibits increased stability relative to other connective arrangements with the same sets of atoms. Generally, aromatic rings contain a set of covalently bound co-planar atoms and comprises a number of 7-electrons (for example, alternating double and single bonds) that is even but not a multiple of 4 (i.e., 4n+2 π-electrons, where n=0, 1, 2, 3, etc.). Aromatic rings include, but are not limited to, phenyl, naphthenyl, imidazolyl, pyrrolyl, pyridinyl, pyrimidinyl, pyrazinyl, pyridonyl, pyridazinyl, pyrimidonyl. Unless stated otherwise specifically in the specification, an “aromatic ring” includes all radicals that are optionally substituted.

“Aryl” refers to a carbocyclic ring system radical comprising 6 to 18 carbon atoms, for example 6 to 10 carbon atoms (C₆-C₁₀ aryl) and at least one carbocyclic aromatic ring. For purposes of embodiments of this disclosure, the aryl radical is a monocyclic, bicyclic, tricyclic or tetracyclic ring system, which may include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl group is optionally substituted.

“Carbocyclic” or “carbocycle” refers to a ring system, wherein each of the ring atoms are carbon.

“Cycloalkyl” refers to a non-aromatic monocyclic or polycyclic carbocyclic radical consisting solely of carbon and hydrogen atoms, which may include fused or bridged ring systems, having from three to fifteen ring carbon atoms (C₃-C₁₅ cycloalkyl), from three to ten ring carbon atoms (C₃-C₁₀ cycloalkyl), or from three to eight ring carbon atoms (C₃-C₈ cycloalkyl), or any value within these ranges such as three to four carbon atoms (C₃-C₄ cycloalkyl), and which is saturated or partially unsaturated and attached to the rest of the molecule by a single bond. Monocyclic radicals include, for example, cyclopropyl, cyclobutyl, cyclopentyl, cyclohexyl, cycloheptyl, and cyclooctyl. Polycyclic radicals include, for example, adamantyl, norbornyl, decalinyl, 7,7-dimethyl-bicyclo[2.2.1]heptanyl, and the like. Unless otherwise stated specifically in the specification, a cycloalkyl group is optionally substituted.

“Fused” refers to any ring structure described herein which is fused to another ring structure.

“Halo” refers to bromo, chloro, fluoro, or iodo.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group is optionally substituted.

“Halocycloalkyl” refers to a cycloalkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a halocycloalkyl group is optionally substituted.

“Hydroxylalkyl” refers to an alkyl radical, as defined above that is substituted by one or more hydroxyl radical. The hydroxyalkyl radical is joined at the main chain through the alkyl carbon atom. Unless stated otherwise specifically in the specification, a hydroxylalkyl group is optionally substituted.

“Heterocyclyl” refers to a 3- to 18-membered, for example 3- to 10-membered or 3- to 8-membered, non-aromatic ring radical having one to ten ring carbon atoms (e.g., two to ten) and from one to six ring heteroatoms selected from the group consisting of nitrogen, oxygen and sulfur. Unless stated otherwise specifically in the specification, the heterocyclyl radical is partially or fully saturated and is a monocyclic, bicyclic, tricyclic or tetracyclic ring system, which may include fused, spirocyclic and/or bridged ring systems. Nitrogen, carbon and sulfur atoms in a heterocyclyl radical are optionally oxidized, and nitrogen atoms may be optionally quaternized. Examples of such heterocyclyl radicals include, but are not limited to, dioxolanyl, thienyl[1,3]dithianyl, decahydroisoquinolyl, furanonyl, imidazolinyl, imidazolidinyl, isothiazolidinyl, isoxazolidinyl, morpholinyl, octahydroindolyl, octahydroisoindolyl, hexahydro-1H-pyrrolizine, 2-oxopiperazinyl, 2-oxopiperidinyl, 2-oxopyrrolidinyl, oxazolidinyl, oxiranyl, piperidinyl, piperazinyl, 4-piperidonyl, azetidinyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group is optionally substituted.

“Heterocyclylalkyl” refers to an alkyl radical, as defined above that is substituted by a heterocyclyl radical. Unless stated otherwise specifically in the specification, a heterocyclylalkyl group is optionally substituted.

“Heteroaryl” refers to a 5- to 18-membered, for example 5- to 6-membered, ring system radical comprising one to thirteen ring carbon atoms, one to six ring heteroatoms selected from the group consisting of nitrogen, oxygen and sulfur, and at least one aromatic ring. Heteroaryl radicals may be a monocyclic, bicyclic, tricyclic or tetracyclic ring system, which may include fused or bridged ring systems; and the nitrogen, carbon or sulfur atoms in the heteroaryl radical may be optionally oxidized; the nitrogen atom may be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl group is optionally substituted.

The term “substituted” as used herein means any of the above groups (e.g., alkyl, alkenyl, alkylene, alkylcarbonyl, alkoxy, alkoxyalkyl, aminylalkyl, aryl, cyanoalkyl, cycloalkyl, haloalkyl, heterocyclyl, heterocyclene, heterocyclylalkyl, heteroaryl, heteroarylalkyl and/or hydroxylalkyl) wherein at least one hydrogen atom (e.g., 1, 2, 3 or all hydrogen atoms) is replaced by a bond to a non-hydrogen substituent. Examples of non-hydrogen substituents include, but are not limited to: amino, carboxyl, cyano, hydroxyl, halo, nitro, oxo, thiol, thioxo, alkyl, alkenyl, alkylcarbonyl, alkoxy, aryl, cyanoalkyl, cycloalkyl, haloalkyl, heterocyclyl, heterocyclylalkyl, heteroaryl, heteroarylalkyl and/or hydroxylalkyl substituents, each of which may also be optionally substituted with one or more of the above substituents.

In some specific embodiments, the optional substitutions are independently selected from the group consisting of halo, hydroxyl, cyano, aminyl, C₁-C₆ alkyl, C₂-C₆ alkenyl, C₂-C₆ alkynyl, C₁-C₆ haloalkyl, C₃-C₅ cycloalkyl, C₃-C₈ halocycloalkyl, C₆-C₁₀ aryl, 5- or 6-membered heteroaryl, C₁-C₆ alkoxy and 3-8 membered heterocyclyl.

The term “effective amount” or “therapeutically effective amount” refers to that amount of a compound described herein that is sufficient to effect the intended application including but not limited to disease treatment, as defined below. The therapeutically effective amount may vary depending upon the intended treatment application (in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of platelet adhesion and/or cell migration. The specific dose will vary depending on the particular compounds chosen, the dosing regimen to be followed, whether it is administered in combination with other compounds, timing of administration, the tissue to which it is administered, and the physical delivery system in which it is carried.

As used herein, “treatment” or “treating” refer to an approach for obtaining beneficial or desired results with respect to a disease, disorder or medical condition including but not limited to a therapeutic effect and/or a prophylactic effect. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the subject, notwithstanding that the subject may still be afflicted with the underlying disorder. A prophylactic effect includes delaying or eliminating the appearance of a disease or condition, delaying or eliminating the onset of symptoms of a disease or condition, slowing, halting, or reversing the progression of a disease or condition, or any combination thereof. In certain embodiments, for prophylactic benefit, the compositions are administered to a subject at risk of developing a particular disease, or to a subject reporting one or more of the physiological symptoms of a disease, even though a diagnosis of this disease may not have been made.

The term “co-administration,” “administered in combination with,” and their grammatical equivalents, as used herein, encompass administration of two or more agents to an animal, including humans, so that both agents and/or their metabolites are present in the subject at the same time. Co-administration includes simultaneous administration in separate compositions, administration at different times in separate compositions, or administration in a composition in which both agents are present.

“Pharmaceutically acceptable salt” includes both acid and base addition salts.

“Pharmaceutically acceptable acid addition salt” refers to those salts which retain the biological effectiveness of the free bases, which are biologically tolerable, or otherwise biologically suitable for administration to the subject. See, generally, S. M. Berge, et al., “Pharmaceutical Salts”, J. Pharm. Sci., 1977, 66:1-19, and Handbook of Pharmaceutical Salts, Properties, Selection, and Use, Stahl and Wermuth, Eds., Wiley-VCH and VHCA, Zurich, 2002. Preferred pharmaceutically acceptable acid addition salts are those that are pharmacologically effective and suitable for contact with the tissues of patients without undue toxicity, irritation, or allergic response. Pharmaceutically acceptable acid addition salts which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness of the free acids, which are biologically tolerable, or otherwise biologically suitable for administration to the subject. See, generally, S. M. Berge, et al., “Pharmaceutical Salts”, J. Pharm. Sci., 1977, 66:1-19, and Handbook of Pharmaceutical Salts, Properties, Selection, and Use, Stahl and Wermuth, Eds., Wiley-VCH and VHCA, Zurich, 2002. Preferred pharmaceutically acceptable base addition salts are those that are pharmacologically effective and suitable for contact with the tissues of patients without undue toxicity, irritation, or allergic response. Pharmaceutically acceptable base addition salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

In some embodiments, pharmaceutically acceptable salts include quaternary ammonium salts such as quaternary amine alkyl halide salts (e.g., methyl bromide).

The terms “antagonist” and “inhibitor” are used interchangeably, and they refer to a compound having the ability to inhibit a biological function of a target protein, whether by inhibiting the activity or expression of the protein, NEK7. Accordingly, the terms “antagonist” and “inhibitors” are defined in the context of the biological role of the target protein. While preferred antagonists herein specifically interact with (e.g., bind to) the target, compounds that inhibit a biological activity of the target protein by interacting with other members of the signal transduction pathway of which the target protein is a member are also specifically included within this definition. A preferred biological activity inhibited by an antagonist is associated with the development, growth, or spread of a tumor.

The term “agonist” as used herein refers to a compound having the ability to initiate or enhance a biological function of a target protein, whether by inhibiting the activity or expression of the target protein. Accordingly, the term “agonist” is defined in the context of the biological role of the target polypeptide. While preferred agonists herein specifically interact with (e.g., bind to) the target, compounds that initiate or enhance a biological activity of the target polypeptide by interacting with other members of the signal transduction pathway of which the target polypeptide is a member are also specifically included within this definition.

“Signal transduction” is a process during which stimulatory or inhibitory signals are transmitted into and within a cell to elicit an intracellular response.

The term “selective inhibition” or “selectively inhibit” refers to a biologically active agent refers to the agent's ability to preferentially reduce the target signaling activity as compared to off-target signaling activity, via direct or indirect interaction with the target.

“Subject” refers to an animal, such as a mammal, for example a human. The methods described herein can be useful in both human therapeutics and veterinary applications. In some embodiments, the subject is a mammal, and in some embodiments, the subject is human.

“Mammal” includes humans and both domestic animals such as laboratory animals and household pets (e.g., cats, dogs, swine, cattle, sheep, goats, horses, rabbits), and non-domestic animals such as wildlife and the like.

“Prodrug” is meant to indicate a compound that may be converted under physiological conditions or by solvolysis to a biologically active compound described herein (e.g., compounds of Structure (I)). Thus, the term “prodrug” refers to a precursor of a biologically active compound that is pharmaceutically acceptable. In some aspects, a prodrug is inactive when administered to a subject, but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein. The term “prodrug” is also meant to include any covalently bonded carriers, which release the active compound in vivo when such prodrug is administered to a mammalian subject. Prodrugs of an active compound, as described herein, are typically prepared by modifying functional groups present in the active compound in such a way that the modifications are cleaved, either in routine manipulation or in vivo, to the parent active compound. Prodrugs include compounds wherein a hydroxy, amino or thiol group is bonded to any group that, when the prodrug of the active compound is administered to a mammalian subject, cleaves to form a free hydroxy, free amino or free mercapto group, respectively. Examples of prodrugs include, but are not limited to, acetate, formate and benzoate derivatives of a hydroxy functional group, or acetamide, formamide and benzamide derivatives of an amine functional group in the active compound and the like.

The term “in vivo” refers to an event that takes place in a subject's body.

Embodiments disclosed herein are also meant to encompass all pharmaceutically acceptable compounds of Structure (I).

Certain embodiments are also meant to encompass the in vivo metabolic products of the disclosed compounds. Such products may result from, for example, the oxidation, reduction, hydrolysis, amidation, esterification, and the like of the administered compound, primarily due to enzymatic processes. Accordingly, embodiments include compounds produced by a process comprising administering a compound of this disclosure to a mammal for a period of time sufficient to yield a metabolic product thereof. Such products are typically identified by administering a radiolabeled compound of the disclosure in a detectable dose to an animal, such as rat, mouse, guinea pig, monkey, or to human, allowing sufficient time for metabolism to occur, and isolating its conversion products from the urine, blood or other biological samples.

“Stable compound” and “stable structure” are meant to indicate a compound that is sufficiently robust to survive isolation to a useful degree of purity from a reaction mixture, and formulation into an efficacious therapeutic agent.

Often crystallizations produce a solvate of the compounds disclosed herein. As used herein, the term “solvate” refers to an aggregate that comprises one or more compounds of the disclosure with one or more molecules of solvent. In some embodiments, the solvent is water, in which case the solvate is a hydrate. Alternatively, in other embodiments, the solvent is an organic solvent. Thus, the compounds of the present disclosure may exist as a hydrate, including a monohydrate, dihydrate, hemihydrate, sesquihydrate, trihydrate, tetrahydrate and the like, as well as the corresponding solvated forms. In some aspects, the compounds of the disclosure are a true solvate, while in other cases, the compounds of the disclosure merely retain adventitious water or is a mixture of water plus some adventitious solvent.

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution.

A “pharmaceutical composition” refers to formulations of compounds of the disclosure and a medium generally accepted in the art for the delivery of compounds of the disclosure to mammals, e.g., humans. Such a medium includes all pharmaceutically acceptable carriers, diluents or excipients therefor.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier.

A “stereoisomer” refers to a compound made up of the same atoms bonded by the same bonds but having different three-dimensional structures, which are not interchangeable. The present disclosure contemplates various stereoisomers and mixtures thereof and includes “enantiomers”, which refers to two stereoisomers whose molecules are non-superimposable mirror images of one another.

The compounds of the disclosure (i.e., compounds of Structure (I)) or their pharmaceutically acceptable salts may contain one or more centers of geometric asymmetry and may thus give rise to stereoisomers such as enantiomers, diastereomers, and other stereoisomeric forms that are defined, in terms of absolute stereochemistry, as (R)- or (S)- or, as (D)- or (L)- for amino acids. Embodiments thus include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

Embodiments of the present disclosure include all manner of rotamers and conformationally restricted states of a compound of the disclosure. Atropisomers, which are stereoisomers arising because of hindered rotation about a single bond, where energy differences due to steric strain or other contributors create a barrier to rotation that is high enough to allow for isolation of individual conformers, are also included. As an example, certain compounds of the disclosure may exist as mixtures of atropisomers or purified or enriched for the presence of one atropisomer.

In some embodiments, the compounds of Structure (I) are a mixture of enantiomers or diastereomers. In other embodiments, the compounds of Structure (I) are substantially one enantiomer or diastereomer.

A “tautomer” refers to a proton shift from one atom of a molecule to another atom of the same molecule. Embodiments thus include tautomers of the disclosed compounds.

The chemical naming protocol and structure diagrams used herein are a modified form of the I.U.P.A.C. nomenclature system, using the ACD/Name Version 9.07 software program and/or ChemDraw Professional Version 17.0.0.206 software naming program (CambridgeSoft). For complex chemical names employed herein, a substituent group is typically named before the group to which it attaches. For example, cyclopropylethyl comprises an ethyl backbone with a cyclopropyl substituent. Except as described below, all bonds are identified in the chemical structure diagrams herein, except for all bonds on some carbon atoms, which are assumed to be bonded to sufficient hydrogen atoms to complete the valency.

### Compounds

The disclosure provides compounds including pharmaceutically acceptable salts, stereoisomers and prodrugs thereof, which are useful for treating various diseases (e.g., cancer).

Embodiments of the present disclosure provide a compound having the following Structure (I):

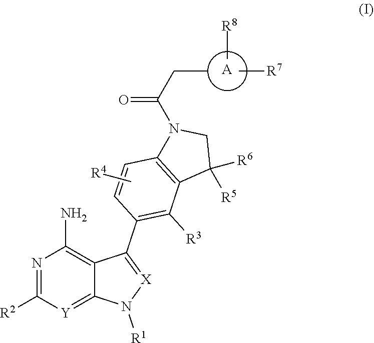

or a pharmaceutically acceptable salt, stereoisomer or prodrug thereof, wherein:
A is C₆-C₁₀ aryl or 5- or 6-membered heteroaryl;
X is N or CR⁹;
Y is N or CH;
R¹ is C₂-C₆ alkyl, C₂-C₆ alkenyl, C₂-C₆ alkynyl, C₃-C₈ cycloalkyl, 3-8 membered heterocyclyl, or 5- or 6-membered heteroaryl, each of which is optionally substituted with one more substituents selected from halo, hydroxyl, cyano, aminyl, carboxy, C₁-C₆ alkyl, C₂-C₆ alkenyl, C₂-C₆ alkynyl, C₁-C₆ alkoxy, and 3-8 membered heterocyclyl;
R² is H or C₁-C₆ alkyl;
R³ is halo;
R⁴ is H, halo, or C₁-C₆ alkyl;
R⁵ and R⁶ are each independently H, halo, C₁-C₆ alkyl or C₁-C₆ haloalkyl, or R⁵ and R⁶, together with the carbon to which they are attached, form a C₃-C₈ cycloalkyl or 3-8 membered heterocyclyl;
R⁷ is C₁-C₆ alkyl, halo, C₁-C₆ haloalkyl, C₁-C₆ haloalkoxy, 3-8 membered heterocyclyl, or 3-8 membered heterocyclylalkyl;
R⁸ is H, halo, C₁-C₆ alkyl, C₁-C₆ haloalkyl, or C₁-C₆ haloalkoxy; and
R⁹ is H, C₁-C₆ alkyl, C₁-C₆ haloalkyl, C₃-C₈ cycloalkyl, or 3-8 membered heterocyclyl.

Embodiments of the present disclosure provide a compound having the following Structure (I):

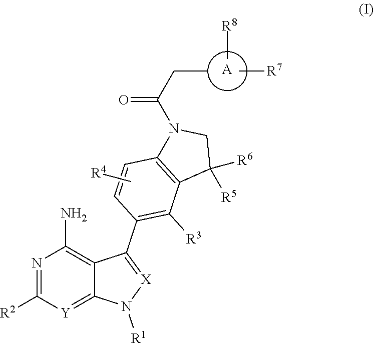

or a pharmaceutically acceptable salt, stereoisomer or prodrug thereof, wherein:
A is C₆-C₁₀ aryl or 5- or 6-membered heteroaryl;
X is N or CR⁹;
Y is N or CH;
R¹ is H, C₂-C₆ alkyl, C₂-C₆ alkenyl, C₂-C₆ alkynyl, C₃-C₈ cycloalkyl, 3-8 membered heterocyclyl, or 5- or 6-membered heteroaryl, each of which is optionally substituted with one more substituents selected from halo, hydroxyl, cyano, aminyl, carboxy, C₁-C₆ alkyl, C₂-C₆ alkenyl, C₂-C₆ alkynyl, C₁-C₆ alkoxy, and 3-8 membered heterocyclyl;
R² is H or C₁-C₆ alkyl;
R³ is halo;
R⁴ is H, halo, or C₁-C₆ alkyl;
R⁵ and R⁶ are each independently H, halo, C₁-C₆ alkyl or C₁-C₆ haloalkyl, or R⁵ and R⁶, together with the carbon to which they are attached, form a C₃-C₈ cycloalkyl or 3-8 membered heterocyclyl;
R⁷ is C₁-C₆ alkyl, halo, C₁-C₆ haloalkyl, C₁-C₆ haloalkoxy, 3-8 membered heterocyclyl, or 3-8 membered heterocyclylalkyl;
R⁸ is H, halo, C₁-C₆ alkyl, C₁-C₆ haloalkyl, or C₁-C₆ haloalkoxy; and
R⁹ is H, C₁-C₆ alkyl, C₁-C₆ haloalkyl, C₃-C₈ cycloalkyl, or 3-8 membered heterocyclyl.

In some embodiments, X is CR⁹. In certain embodiments, R⁹ is H. In some more specific embodiments, R⁹ is C₁-C₆ alkyl, C₁-C₆ haloalkyl, C₃-C₈ cycloalkyl, or 3-8 membered heterocyclyl. In some other embodiments, X is N.

In more embodiments, Y is N. In other embodiments, Y is CH.

In some specific embodiments, A is C₆-C₁₀ aryl. In more specific embodiments, A is phenyl. In certain embodiments, Y is 5- or 6-membered heteroaryl. In more specific embodiments, Y is pyridinyl. In some embodiments, Y is pyrimidinyl. In some embodiments, Y is furanyl, thiophenyl, thiazolyl, oxazolyl, isoxazolyl, pyrrolyl, or imidazolyl.

In certain embodiments, R¹ is C₂-C₆ alkyl. In some embodiments, R¹ is C₃-C₆ alkyl. In certain related embodiments, R¹ is isopropyl or —CH₂CH₂OH. In certain embodiments, R¹ has one of the following structures:

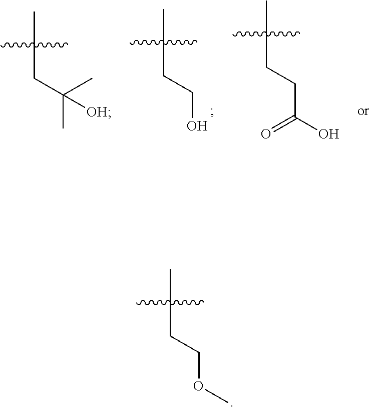

In certain embodiments, R¹ has the following structure:

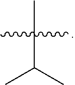 [SMILES: *C(C)C]

In some embodiments, R¹ is C₃-C₈ cycloalkyl. In certain related embodiments, R¹ is cyclopropyl or cyclobutyl. In some embodiments, R¹ has one of the following structures:

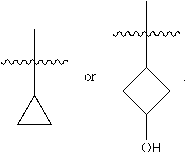

In some embodiments, R¹ is a 3-8 membered heterocyclyl. In more specific embodiments, R¹ is piperidinyl, azetidinyl, oxetanyl, or tetrapyranyl. In some specific embodiments, R¹ has one of the following structures:

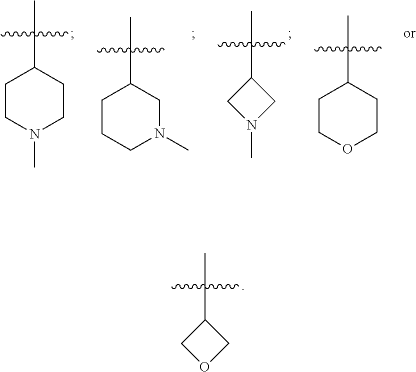

In some other embodiments, R¹ is C₂-C₆ alkenyl, C₂-C₆ alkynyl, or 5- or 6-membered heteroaryl.

In some embodiments, R¹ is substituted with one more substituents selected from halo, hydroxyl, cyano, aminyl, carboxy, C₁-C₆ alkyl, C₂-C₆ alkenyl, C₂-C₆ alkynyl, C₁-C₆ alkoxy, and 3-8 membered heterocyclyl. In other embodiments, R¹ is unsubstituted. In some embodiments, R¹ is H.

In some embodiments, R¹ is H or has one of the following structures:

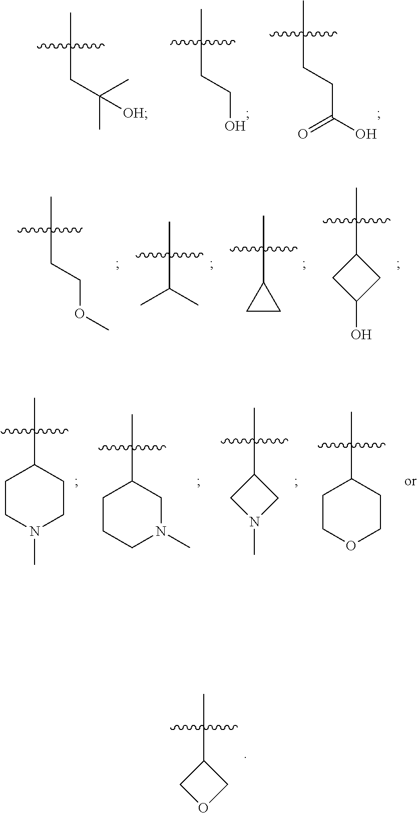

In some embodiments, R² is H. In certain embodiments, R² is C₁-C₆ alkyl.

In some embodiments, R³ is fluoro. In more embodiments, R³ is chloro. In certain embodiments, R³ is bromo or iodo.

In certain embodiments, R⁴ is H. In some embodiments, R⁴ is halo or C₁-C₆ alkyl.

In certain specific embodiments, R⁵ is H. In some embodiments, R⁵ is halo, C₁-C₆ alkyl, or C₁-C₆ haloalkyl. In some embodiments, R⁶ is H. In some specific embodiments, R⁶ is halo, C₁-C₆ alkyl, or C₁-C₆ haloalkyl. In certain embodiments, R⁵ and R⁶, together with the carbon to which they are attached, form a C₃-C₈ cycloalkyl or 3-8 membered heterocyclyl.

In some embodiments, R⁷ is C₁-C₆ haloalkyl. In certain embodiments, R⁷ is trifluoromethyl. In some specific embodiments, R⁷ is halo. In certain specific embodiments, R⁷ is fluoro. In some more specific embodiments, R⁷ is chloro, bromo, or iodo. In certain more specific embodiments, R⁷ is C₁-C₆ haloalkoxy. In some embodiments, R⁷ is trifluoromethoxy. In more specific embodiments, R⁷ is C₁-C₆ alkyl. In more specific embodiments, R⁷ is methyl. In some embodiments, R⁷ is 3-8 membered heterocyclylalkyl. In more specific embodiments, R⁷ has the following structure:

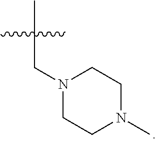 [SMILES: *CN1CCN(C)CC1]

In certain embodiments, R⁸ is H. In some embodiments, R⁸ is C₁-C₆ haloalkyl. In certain specific embodiments, R⁸ is trifluoromethyl. In some specific embodiments, R⁸ is C₁-C₆ alkyl. In certain more specific embodiments, R⁸ is methyl. In some more specific embodiments, R⁸ is halo. In some embodiments, R⁸ is fluoro. In other embodiments, R⁸ is bromo, chloro, or iodo.

In some specific embodiments, A has one of the following structures:

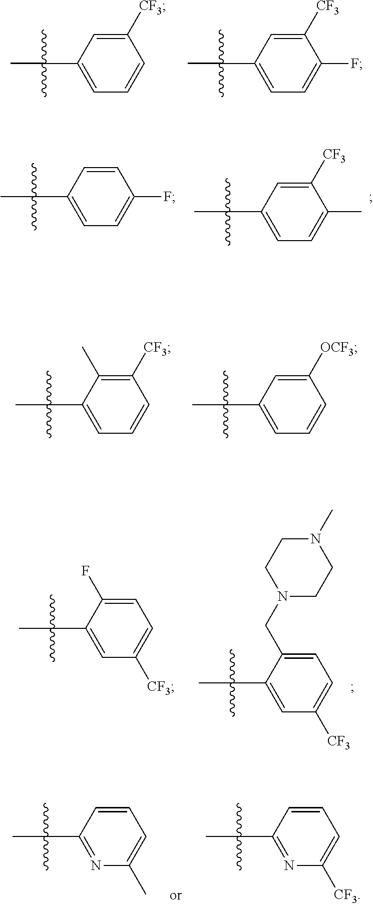

In some specific embodiments, A has one of the following structures:

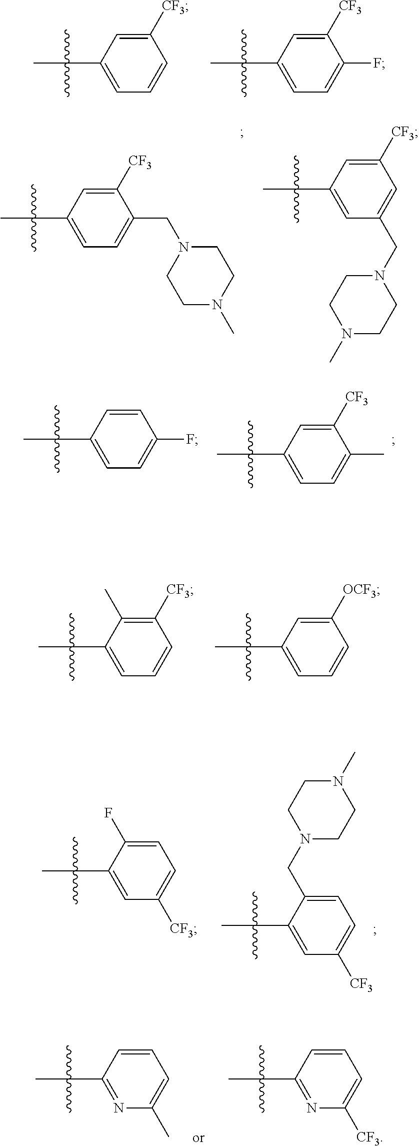

In a specific embodiment, the compound of Structure (I) is an inhibitor of NEK7 in a patient or in a biological sample. In some embodiments, the compound of Structure (I) is a modulator of the NLRP3 inflammasome.

In various different embodiments, the compound has one of the structures set forth in Table 1 below, or a pharmaceutically acceptable salt, stereoisomer, or prodrug thereof. Compounds in Table 1 were prepared as described in the Examples or methods known in the art and analyzed by mass spectrometry and/or ¹H NMR.

TABLE 1
Representative compounds of Structure (I)

| Compound | Structure | Name |
|---|---|---|
| I-1 | 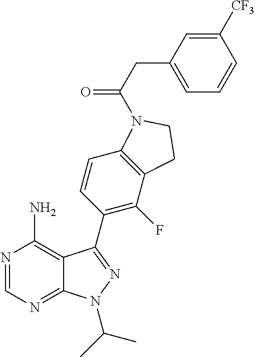 [SMILES: CC(C)n1nc(-c2ccc3c(c2F)CCN3C(=O)Cc2cccc(C(F)(F)F)c2)c2c(N)ncnc21] | 1-(5-(4-amino-1-isoprop- yl-1H-pyrazolo[3,4-d]- pyrimidin-3-yl)-4-fluoro- indolin-1-yl)-2-(3-(tri- fluoromethyl)phenyl)- ethan-1-one |
| I-2 | 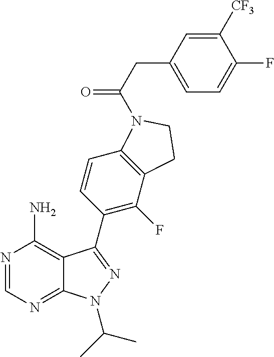 [SMILES: CC(C)n1nc(-c2ccc3c(c2F)CCN3C(=O)Cc2ccc(F)c(C(F)(F)F)c2)c2c(N)ncnc21] | 1-(5-(4-amino-1-isoprop- yl-1H-pyrazolo[3,4-d]- pyrimidin-3-yl)-4-fluoro- indolin-1-yl)-2-(4-fluoro- 3-(trifluoromethyl)phen- yl)ethan-1-one |
| I-3 | 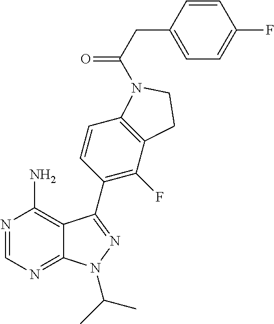 [SMILES: CC(C)n1nc(-c2ccc3c(c2F)CCN3C(=O)Cc2ccc(F)cc2)c2c(N)ncnc21] | 1-(5-(4-amino-1-iso- propyl-1H-pyrazolo[3,4- d]pyrimidin-3-yl)-4- fluoroindolin-1-yl)-2-(4- fluorophenyl)ethan-1- one |
| I-4 | 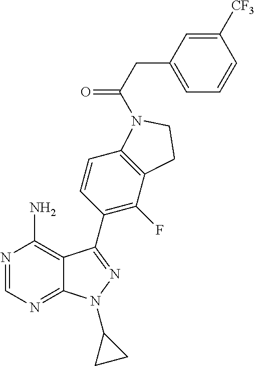 [SMILES: Nc1ncnc2c1c(-c1ccc3c(c1F)CCN3C(=O)Cc1cccc(C(F)(F)F)c1)nn2C1CC1] | 1-(5-(4-amino-1-cyclo- propyl-1H-pyrazolo[3,4- d]pyrimidin-3-yl)-4- fluoroindolin-1-yl)-2-(3- (trifluoromethyl)phenyl)- ethan-1-one |
| I-5 | 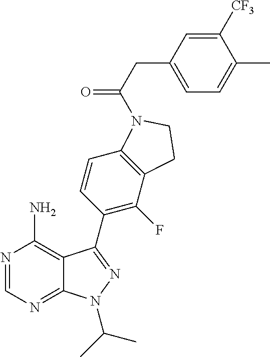 [SMILES: Cc1ccc(CC(=O)N2CCc3c2ccc(-c2nn(C(C)C)c4ncnc(N)c24)c3F)cc1C(F)(F)F] | 1-(5-(4-amino-1-iso- propyl-1H-pyrazolo[3,4- d]pyrimidin-3-yl)-4- fluoroindolin-1-yl)-2-(4- methyl-3-(trifluorometh- yl)phenyl)ethan-1-one |
| I-6 | 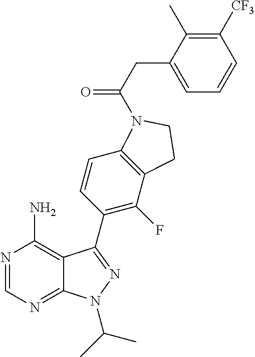 [SMILES: Cc1c(CC(=O)N2CCc3c2ccc(-c2nn(C(C)C)c4ncnc(N)c24)c3F)cccc1C(F)(F)F] | 1-(5-(4-amino-1-iso- propyl-1H-pyrazolo[3,4- d]pyrimidin-3-yl)-4- fluoroindolin-1-yl)-2- (2-methyl-3-(trifluoro- methyl)phenyl)ethan-1- one |
| I-7 | 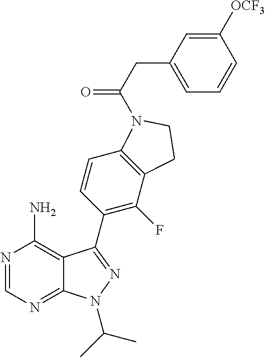 [SMILES: CC(C)n1nc(-c2ccc3c(c2F)CCN3C(=O)Cc2cccc(OC(F)(F)F)c2)c2c(N)ncnc21] | 1-(5-(4-amino-1-iso- propyl-1H-pyrazolo[3,4- d]pyrimidin-3-yl)-4- fluoroindolin-1-yl)-2-(3- (trifluoromethoxy)phen- yl)ethan-1-one |
| I-8 | 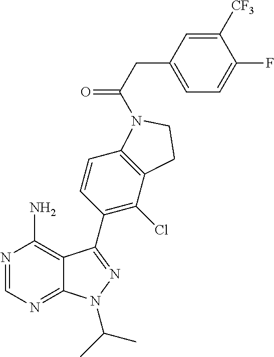 [SMILES: CC(C)n1nc(-c2ccc3c(c2Cl)CCN3C(=O)Cc2ccc(F)c(C(F)(F)F)c2)c2c(N)ncnc21] | 1-(5-(4-amino-1-iso- propyl-1H-pyrazolo[3,4- d]pyrimidin-3-yl)-4- chloroindolin-1-yl)-2-(4- fluoro-3-(trifluorometh- yl)phenyl)ethan-1-one |
| I-9 | 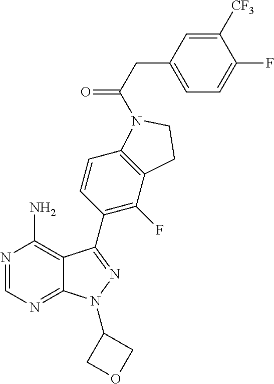 [SMILES: Nc1ncnc2c1c(-c1ccc3c(c1F)CCN3C(=O)Cc1ccc(F)c(C(F)(F)F)c1)nn2C1COC1] | 1-(5-(4-amino-1-(oxetan- 3-yl)-1H-pyrazolo[3,4- d]pyrimidin-3-yl)-4- fluoroindolin-1-yl)-2-(4- fluoro-3-(trifluorometh- yl)phenyl)ethan-1-one |
| I-10 | 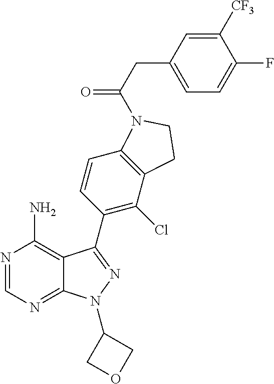 [SMILES: Nc1ncnc2c1c(-c1ccc3c(c1Cl)CCN3C(=O)Cc1ccc(F)c(C(F)(F)F)c1)nn2C1COC1] | 1-(5-(4-amino-1-(oxetan- 3-yl)-1H-pyrazolo[3,4- d]pyrimidin-3-yl)-4- chloroindolin-1-yl)-2-(4- fluoro-3-(trifluorometh- yl)phenyl)ethan-1-one |
| I-11 | 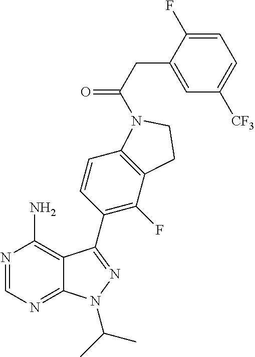 [SMILES: CC(C)n1nc(-c2ccc3c(c2F)CCN3C(=O)Cc2cc(C(F)(F)F)ccc2F)c2c(N)ncnc21] | 1-(5-(4-amino-1-isoprop- yl-1H-pyrazolo[3,4-d]- pyrimidin-3-yl)-4-fluoro- indolin-1-yl)-2-(2-fluoro- 5-(trifluoromethyl)phen- yl)ethan-1-one |
| I-12 | 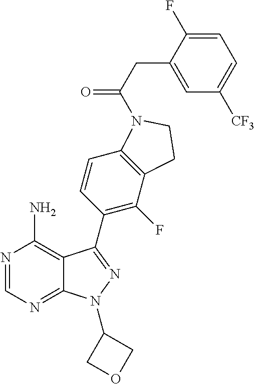 [SMILES: Nc1ncnc2c1c(-c1ccc3c(c1F)CCN3C(=O)Cc1cc(C(F)(F)F)ccc1F)nn2C1COC1] | 1-(5-(4-amino-1-(oxetan- 3-yl)-1H-pyrazolo[3,4- d]pyrimidin-3-yl)-4- fluoroindolin-1-yl)-2-(2- fluoro-5-(trifluorometh- yl)phenyl)-ethan-1-one |
| I-13 | 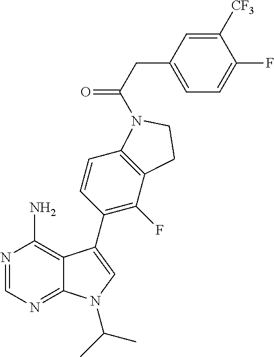 [SMILES: CC(C)n1cc(-c2ccc3c(c2F)CCN3C(=O)Cc2ccc(F)c(C(F)(F)F)c2)c2c(N)ncnc21] | 1-(5-(4-amino-7-isoprop- yl-7H-pyrrolo[2,3-d]pyr- imidin-5-yl)-4-fluoroind- olin-1-yl)-2-(4-fluoro-3- (trifluoromethyl)phenyl)- ethan-1-one |
| I-14 | 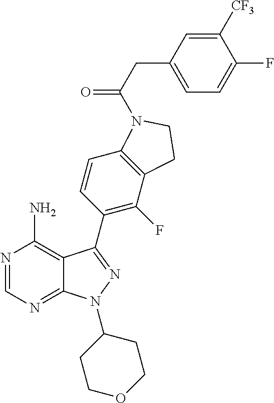 [SMILES: Nc1ncnc2c1c(-c1ccc3c(c1F)CCN3C(=O)Cc1ccc(F)c(C(F)(F)F)c1)nn2C1CCOCC1] | 1-(5-(4-amino-1-(tetra- hydro-2H-pyran-4-yl)- 1H-pyrazolo[3,4-d]pyr- imidin-3-yl)-4-fluoro- indolin-1-yl)-2-(4-fluoro- 3-(trifluoromethyl)phen- yl)ethan-1-one |
| I-15 | 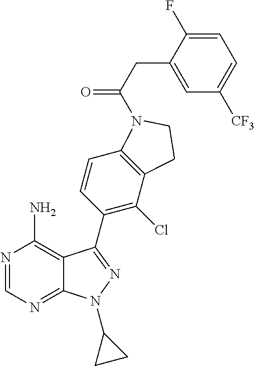 [SMILES: Nc1ncnc2c1c(-c1ccc3c(c1Cl)CCN3C(=O)Cc1cc(C(F)(F)F)ccc1F)nn2C1CC1] | 1-(5-(4-amino-1-cyclo- propyl-1H-pyrazolo[3,4- d]pyrimidin-3-yl)-4- chloroindolin-1-yl)-2- (2-fluoro-5-(trifluoro- methyl)phenyl)ethan-1- one |
| I-16 | 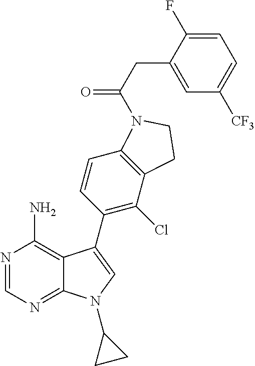 [SMILES: Nc1ncnc2c1c(-c1ccc3c(c1Cl)CCN3C(=O)Cc1cc(C(F)(F)F)ccc1F)cn2C1CC1] | 1-(5-(4-amino-7-cyclo- propyl-7H-pyrrolo[2,3- d]pyrimidin-5-yl)-4- chloroindolin-1-yl)-2- (2-fluoro-5-(trifluoro- methyl)phenyl)ethan-1- one |
| I-17 | 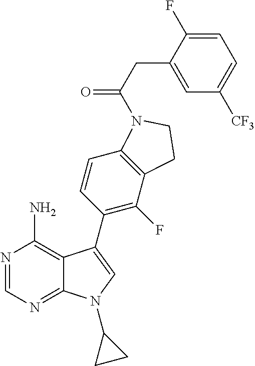 [SMILES: Nc1ncnc2c1c(-c1ccc3c(c1F)CCN3C(=O)Cc1cc(C(F)(F)F)ccc1F)cn2C1CC1] | 1-(5-(4-amino-7-cyclo- propyl-7H-pyrrolo[2,3- d]pyrimidin-5-yl)-4- fluoroindolin-1-yl)-2-(2- fluoro-5-(trifluorometh- yl)phenyl)ethan-1-one |
| I-18 | 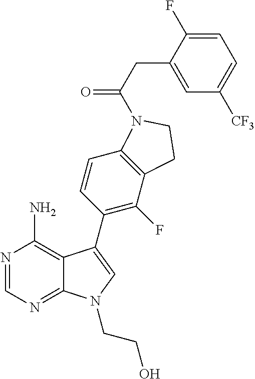 [SMILES: Nc1ncnc2c1c(-c1ccc3c(c1F)CCN3C(=O)Cc1cc(C(F)(F)F)ccc1F)cn2CCO] | 1-(5-(4-amino-7-(2- hydroxyethyl)-7H-pyr- rolo[2,3-d]pyrimidin-5- yl)-4-fluoroindolin-1- yl)-2-(2-fluoro-5-(tri- fluoromethyl)phenyl)- ethan-1-one |
| I-19 | 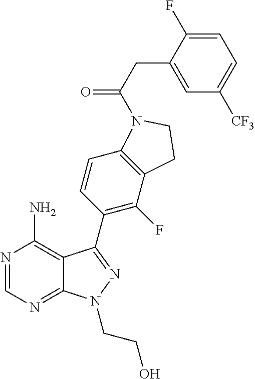 [SMILES: Nc1ncnc2c1c(-c1ccc3c(c1F)CCN3C(=O)Cc1cc(C(F)(F)F)ccc1F)nn2CCO] | 1-(5-(4-amino-1-(2- hydroxyethyl)-1H-pyr- azolo[3,4-d]pyrimidin- 3-yl)-4-fluoroindolin-1- yl)-2-(2-fluoro-5-(tri- fluoromethyl)phenyl)- ethan-1-one |
| I-20 | 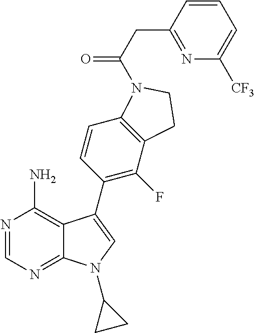 [SMILES: Nc1ncnc2c1c(-c1ccc3c(c1F)CCN3C(=O)Cc1cccc(C(F)(F)F)n1)cn2C1CC1] | 1-(5-(4-amino-7-cyclo- propyl-7H-pyrrolo[2,3- d]pyrimidin-5-yl)-4- fluoroindolin-1-yl)-2- (6-(trifluoromethyl)pyr- idin-2-yl)ethan-1-one |
| I-21 | 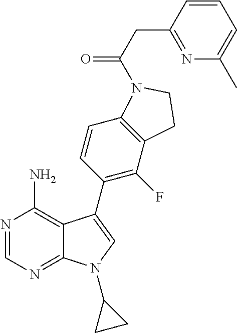 [SMILES: Cc1cccc(CC(=O)N2CCc3c2ccc(-c2cn(C4CC4)c4ncnc(N)c24)c3F)n1] | 1-(5-(4-amino-7-cyclo- propyl-7H-pyrrolo[2,3- d]pyrimidin-5-yl)-4- fluoroindolin-1-yl)-2- (6-methylpyridin-2-yl)- ethan-1-one |
| I-22 | 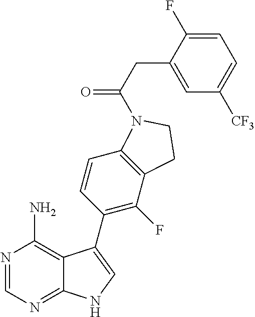 [SMILES: Nc1ncnc2[nH]cc(-c3ccc4c(c3F)CCN4C(=O)Cc3cc(C(F)(F)F)ccc3F)c12] | 1-(5-(4-amino-7H-pyr- rolo[2,3-d]pyrimidin-5- yl)-4-fluoroindolin-1- yl)-2-(2-fluoro-5-(tri- fluoromethyl)phenyl)- ethan-1-one |
| I-23 | 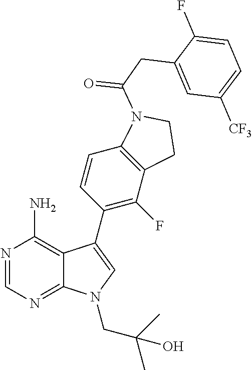 [SMILES: CC(C)(O)Cn1cc(-c2ccc3c(c2F)CCN3C(=O)Cc2cc(C(F)(F)F)ccc2F)c2c(N)ncnc21] | 1-(5-(4-amino-7-(2- hydroxy-2-methylprop- yl)-7H-pyrrolo[2,3-d]- pyrimidin-5-yl)-4- fluoroindolin-1-yl)-2- (2-fluoro-5-(trifluoro- methyl)phenyl)ethan- 1-one |
| I-24 | 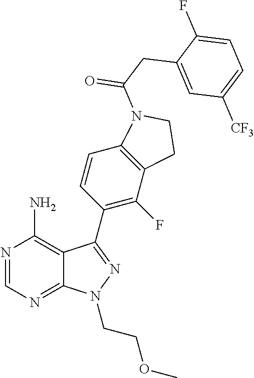 [SMILES: COCCn1nc(-c2ccc3c(c2F)CCN3C(=O)Cc2cc(C(F)(F)F)ccc2F)c2c(N)ncnc21] | 1-(5-(4-amino-1-(2- methoxyethyl)-1H- pyrazolo[3,4-d]pyrim- idin-3-yl)-4-fluoroindo- lin-1-yl)-2-(2-fluoro-5- (trifluoromethyl)phen- yl)ethan-1-one |
| I-25 | 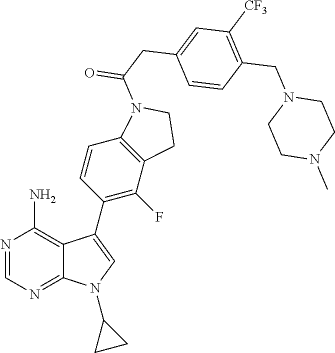 [SMILES: CN1CCN(Cc2ccc(CC(=O)N3CCc4c3ccc(-c3cn(C5CC5)c5ncnc(N)c35)c4F)cc2C(F)(F)F)CC1] | 1-(5-(4-amino-7-cyclo- propyl-7H-pyrrolo[2,3- d]pyrimidin-5-yl)-4- fluoroindolin-1-yl)-2- (4-((4-methylpiperazin- 1-yl)methyl)-3-(tri- fluoromethyl)phenyl)- ethan-1-one |
| I-26 | 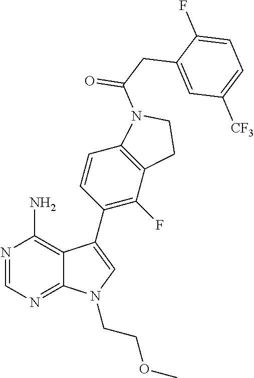 [SMILES: COCCn1cc(-c2ccc3c(c2F)CCN3C(=O)Cc2cc(C(F)(F)F)ccc2F)c2c(N)ncnc21] | 1-(5-(4-amino-7-(2- methoxyethyl)-7H-pyr- rolo[2,3-d]pyrimidin-5- yl)-4-fluoroindolin-1- yl)-2-(2-fluoro-5-(tri- fluoromethyl)phenyl)- ethan-1-one |
| I-27 | 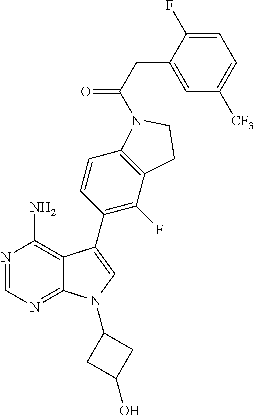 [SMILES: Nc1ncnc2c1c(-c1ccc3c(c1F)CCN3C(=O)Cc1cc(C(F)(F)F)ccc1F)cn2C1CC(O)C1] | 1-(5-(4-amino-7-(3- hydroxycyclobutyl)-7H- pyrrolo[2,3-d]pyrimidin- 5-yl)-4-fluoroindolin-1- yl)-2-(2-fluoro-5-(tri- fluoromethyl)phenyl)- ethan-1-one |
| I-28 | 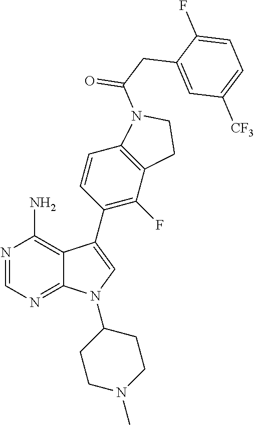 [SMILES: CN1CCC(n2cc(-c3ccc4c(c3F)CCN4C(=O)Cc3cc(C(F)(F)F)ccc3F)c3c(N)ncnc32)CC1] | 1-(5-(4-amino-7-(1- methylpiperidin-4-yl)- 7H-pyrrolo[2,3-d]pyr- imidin-5-yl)-4-fluoro- indolin-1-yl)-2-(2- fluoro-5-(trifluorometh- yl)phenyl)ethan-1-one |
| I-29 | 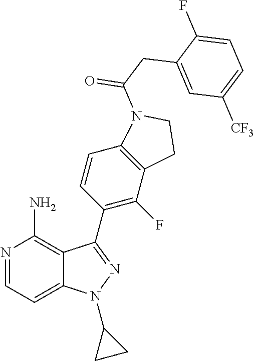 [SMILES: Nc1nccc2c1c(-c1ccc3c(c1F)CCN3C(=O)Cc1cc(C(F)(F)F)ccc1F)nn2C1CC1] | 1-(5-(4-amino-1-cyclo- propyl-1H-pyrazolo[4,3- c]pyridin-3-yl)-4-fluoro- indolin-1-yl)-2-(2-fluoro- 5-(trifluoromethyl)phen- yl)ethan-1-one |
| I-30 | 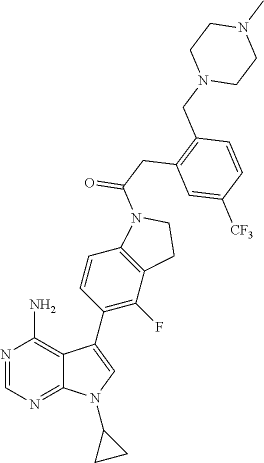 [SMILES: CN1CCN(Cc2ccc(C(F)(F)F)cc2CC(=O)N2CCc3c2ccc(-c2cn(C4CC4)c4ncnc(N)c24)c3F)CC1] | 1-(5-(4-amino-7-cyclo- propyl-7H-pyrrolo[2,3- d]pyrimidin-5-yl)-4- fluoroindolin-1-yl)-2-(2- ((4-methylpiperazin-1- yl)methyl)-5-(trifluoro- methyl)phenyl)ethan-1- one |
| I-31 | 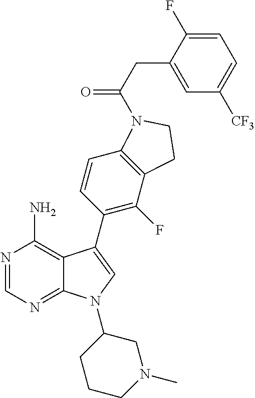 [SMILES: CN1CCCC(n2cc(-c3ccc4c(c3F)CCN4C(=O)Cc3cc(C(F)(F)F)ccc3F)c3c(N)ncnc32)C1] | 1-(5-(4-amino-7-(1- methylpiperidin-3-yl)- 7H-pyrrolo[2,3-d]pyr- imidin-5-yl)-4-fluoroin- dolin-1-yl)-2-(2-fluoro- 5-(trifluoromethyl)phen- yl)ethan-1-one |
| I-32 | 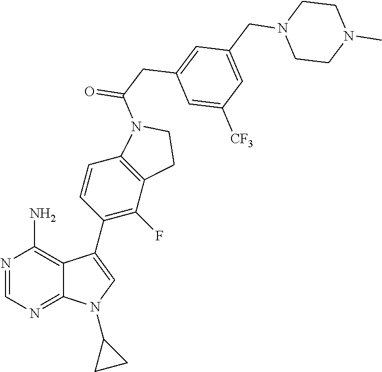 [SMILES: CN1CCN(Cc2cc(CC(=O)N3CCc4c3ccc(-c3cn(C5CC5)c5ncnc(N)c35)c4F)cc(C(F)(F)F)c2)CC1] | 1-(5-(4-amino-7-cyclo- propyl-7H-pyrrolo[2,3- d]pyrimidin-5-yl)-4- fluoroindolin-1-yl)-2- (3-((4-methylpiperazin- 1-yl)methyl)-5-(tri- fluoromethyl)phenyl)- ethan-1-one |
| I-33 | 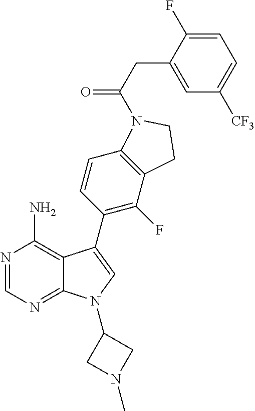 [SMILES: CN1CC(n2cc(-c3ccc4c(c3F)CCN4C(=O)Cc3cc(C(F)(F)F)ccc3F)c3c(N)ncnc32)C1] | 1-(5-(4-amino-7-(1- methylazetidin-3-yl)- 7H-pyrrolo[2,3-d]pyr- imidin-5-yl)-4-fluoroin- dolin-1-yl)-2-(2-fluoro- 5-(trifluoromethyl)phen- yl)ethan-1-one |
| I-34 | 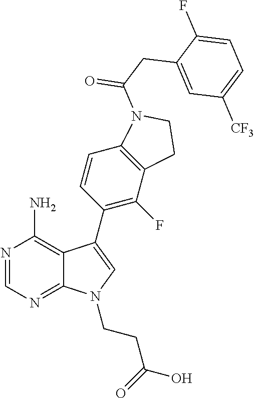 [SMILES: Nc1ncnc2c1c(-c1ccc3c(c1F)CCN3C(=O)Cc1cc(C(F)(F)F)ccc1F)cn2CCC(=O)O] | 3-(4-amino-5-(4-fluoro- 1-(2-(2-fluoro-5-(tri- fluoromethyl)phenyl)- acetyl)indolin-5-yl)-7H- pyrrolo[2,3-d]pyrimidin- 7-yl)propanoic acid |

It is understood that in the present description, combinations of substituents and/or variables of the depicted formulae are permissible only if such contributions result in stable compounds.

In an additional embodiment, various compounds of the disclosure which exist in free base or acid form can be converted to their pharmaceutically acceptable salts by treatment with the appropriate inorganic or organic base or acid by methods known to one skilled in the art. Salts of the compounds of the disclosure can be converted to their free base or acid form by standard techniques.

### Pharmaceutical Compositions

Other embodiments are directed to pharmaceutical compositions. The pharmaceutical composition comprises any one (or more) of the foregoing compounds and a pharmaceutically acceptable carrier. In some embodiments, the pharmaceutical composition is formulated for oral administration. In other embodiments, the pharmaceutical composition is formulated for injection. In still more embodiments, the pharmaceutical compositions comprise a compound as disclosed herein and an additional therapeutic agent (e.g., anticancer agent). Non-limiting examples of such therapeutic agents are described herein below.

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, a compound as described herein is administered in a local rather than systemic manner, for example, via injection of the compound directly into an organ, often in a depot preparation or sustained release formulation. In specific embodiments, long acting formulations are administered by implantation (for example subcutaneously or intramuscularly) or by intramuscular injection. Furthermore, in other embodiments, the compound is delivered in a targeted drug delivery system, for example, in a liposome coated with and organ-specific antibody. In such embodiments, the liposomes are targeted to and taken up selectively by the organ. In yet other embodiments, the compound as described herein is provided in the form of a rapid release formulation, in the form of an extended release formulation, or in the form of an intermediate release formulation. In yet other embodiments, the compound described herein is administered topically.

In treatment methods according to embodiments of the disclosure, an effective amount of at least one compound of Structure (I) is administered to a subject suffering from or diagnosed as having such a disease, disorder, or medical condition. Effective amounts or doses may be ascertained by methods such as modeling, dose escalation studies or clinical trials, e.g., the mode or route of administration or drug delivery, the pharmacokinetics of the agent, the severity and course of the disease, disorder, or condition, the subject's previous or ongoing therapy, the subject's health status and response to drugs, and the judgment of the treating physician.

The compounds according to the disclosure are effective over a wide dosage range. For example, in the treatment of adult humans, dosages from 10 to 5000 mg, from 100 to 5000 mg, from 1000 mg to 4000 mg per day, and from 1000 to 3000 mg per day are examples of dosages that are used in some embodiments. The exact dosage will depend upon the route of administration, the form in which the compound is administered, the subject to be treated, the body weight of the subject to be treated, and the preference and experience of the attending physician.

In some embodiments, compounds of the disclosure are administered in a single dose. Typically, such administration will be by injection, e.g., intravenous injection, in order to introduce the agent quickly. However, other routes are used as appropriate. A single dose of a compound of the disclosure may also be used for treatment of an acute condition.

In some embodiments, compounds of the disclosure are administered in multiple doses. In some embodiments, dosing is about once, twice, three times, four times, five times, six times, or more than six times per day. In other embodiments, dosing is about once a month, once every two weeks, once a week, or once every other day. In another embodiment compounds of the disclosure and another agent (e.g., anti-cancer agent) are administered together about once per day to about 6 times per day. In another embodiment the administration of compounds of the disclosure and an agent continues for less than about 7 days. In yet another embodiment the administration continues for more than about 6, 10, 14, 28 days, two months, six months, or one year. In some cases, continuous dosing is achieved and maintained as long as necessary.

Administration of compounds of the disclosure may continue as long as necessary. In some embodiments, compounds of the disclosure are administered for more than 1, 2, 3, 4, 5, 6, 7, 14, or 28 days. In some embodiments, compounds of the disclosure are administered for less than 28, 14, 7, 6, 5, 4, 3, 2, or 1 day. In some embodiments, compounds of the disclosure are administered chronically on an ongoing basis, e.g., for the treatment of chronic effects.

In some embodiments, the compounds of the disclosure are administered in individual dosage forms. It is known in the art that due to inter-subject variability in compound pharmacokinetics, individualization of dosing regimen is necessary for optimal therapy.

In some embodiments, the compounds described herein are formulated into pharmaceutical compositions. In specific embodiments, pharmaceutical compositions are formulated in a conventional manner using one or more physiologically acceptable carriers comprising excipients and auxiliaries which facilitate processing of the disclosed compounds into preparations which can be used pharmaceutically. Proper formulation is dependent upon the route of administration chosen. Any pharmaceutically acceptable techniques, carriers, and excipients are used as suitable to formulate the pharmaceutical compositions described herein: Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

Provided herein are pharmaceutical compositions comprising one or more compounds of Structure (I), and a pharmaceutically acceptable carrier.

Provided herein are pharmaceutical compositions comprising one or more compounds selected from compounds of Structure (I) and pharmaceutically acceptable diluent(s), excipient(s), and carrier(s). In certain embodiments, the compounds described are administered as pharmaceutical compositions in which one or more compounds selected from compounds of Structure (I) are mixed with other active ingredients, as in combination therapy. Encompassed herein are all combinations of actives set forth in the combination therapies section below and throughout this disclosure. In specific embodiments, the pharmaceutical compositions include one or more compounds of Structure (I).

In a specific embodiment, pharmaceutical compositions of the compounds of Structure (I) inhibit NEK7 when administered to a patient or a biological sample.

A pharmaceutical composition, as used herein, refers to a mixture of one or more compounds selected from compounds of Structure (I) with other chemical components, such as carriers, stabilizers, diluents, dispersing agents, suspending agents, thickening agents, and/or excipients. In certain embodiments, the pharmaceutical composition facilitates administration of the compound to an organism. In some embodiments, therapeutically effective amounts of one or more compounds selected from compounds of Structure (I) provided herein are administered in a pharmaceutical composition to a mammal having a disease, disorder or medical condition to be treated. In specific embodiments, the mammal is a human. In certain embodiments, therapeutically effective amounts vary depending on the severity of the disease, the age and relative health of the subject, the potency of the compound used and other factors. The compounds described herein are used singly or in combination with one or more therapeutic agents as components of mixtures.

In one embodiment, one or more compounds selected from compounds of Structure (I) are formulated in aqueous solutions. In specific embodiments, the aqueous solution is selected from, by way of example only, a physiologically compatible buffer, such as Hank's solution, Ringer's solution, or physiological saline buffer. In other embodiments, one or more compounds selected from compounds of Structure (I) are formulated for trans-mucosal administration. In specific embodiments, trans-mucosal formulations include penetrants that are appropriate to the barrier to be permeated. In still other embodiments wherein the compounds described herein are formulated for other parenteral injections, appropriate formulations include aqueous or non-aqueous solutions. In specific embodiments, such solutions include physiologically compatible buffers and/or excipients.

In another embodiment, compounds described herein are formulated for oral administration. Compounds described herein are formulated by combining the active compounds with, e.g., pharmaceutically acceptable carriers or excipients. In various embodiments, the compounds described herein are formulated in oral dosage forms that include, by way of example only, tablets, powders, pills, dragees, capsules, liquids, gels, syrups, elixirs, slurries, suspensions and the like.

In certain embodiments, pharmaceutical preparations for oral use are obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. In specific embodiments, disintegrating agents are optionally added. Disintegrating agents include, by way of example only, cross-linked croscarmellose sodium, polyvinylpyrrolidone, agar, or alginic acid or a salt thereof such as sodium alginate.

In one embodiment, dosage forms, such as dragee cores and tablets, are provided with one or more suitable coating. In specific embodiments, concentrated sugar solutions are used for coating the dosage form. The sugar solutions, optionally contain additional components, such as by way of example only, gum arabic, talc, polyvinylpyrrolidone, carbopol gel, polyethylene glycol, and/or titanium dioxide, lacquer solutions, and suitable organic solvents or solvent mixtures. Dyestuffs and/or pigments are also optionally added to the coatings for identification purposes. Additionally, the dyestuffs and/or pigments are optionally utilized to characterize different combinations of active compound doses.

In certain embodiments, therapeutically effective amounts of at least one of the compounds described herein are formulated into other oral dosage forms. Oral dosage forms include push-fit capsules made of gelatin, as well as soft, sealed capsules made of gelatin and a plasticizer, such as glycerol or sorbitol. In specific embodiments, push-fit capsules contain the active ingredients in admixture with one or more filler. Fillers include, by way of example only, lactose, binders such as starches, and/or lubricants such as talc or magnesium stearate and, optionally, stabilizers. In other embodiments, soft capsules, contain one or more active compound that is dissolved or suspended in a suitable liquid. Suitable liquids include, by way of example only, one or more fatty oil, liquid paraffin, or liquid polyethylene glycol. In addition, stabilizers are optionally added.

In still other embodiments, the compounds described herein are formulated for parental injection, including formulations suitable for bolus injection or continuous infusion. In specific embodiments, formulations for injection are presented in unit dosage form (e.g., in ampoules) or in multi-dose containers. Preservatives are, optionally, added to the injection formulations. In still other embodiments, the pharmaceutical compositions are formulated in a form suitable for parenteral injection as sterile suspensions, solutions or emulsions in oily or aqueous vehicles. Parenteral injection formulations optionally contain formulatory agents such as suspending, stabilizing and/or dispersing agents. In specific embodiments, pharmaceutical formulations for parenteral administration include aqueous solutions of the active compounds in water-soluble form. In additional embodiments, suspensions of one or more compounds selected from compounds of Structure (I) are prepared as appropriate oily injection suspensions. Suitable lipophilic solvents or vehicles for use in the pharmaceutical compositions described herein include, by way of example only, fatty oils such as sesame oil, or synthetic fatty acid esters, such as ethyl oleate or triglycerides, or liposomes. In certain specific embodiments, aqueous injection suspensions contain substances which increase the viscosity of the suspension, such as sodium carboxymethyl cellulose, sorbitol, or dextran. Optionally, the suspension contains suitable stabilizers or agents which increase the solubility of the compounds to allow for the preparation of highly concentrated solutions. Alternatively, in other embodiments, the active ingredient is in powder form for constitution with a suitable vehicle, e.g., sterile pyrogen-free water, before use.

Pharmaceutical compositions include at least one pharmaceutically acceptable carrier, diluent, or excipient, and one or more compounds selected from compounds of Structure (I), described herein as an active ingredient. The active ingredient is in free-acid or free-base form, or in a pharmaceutically acceptable salt form. In addition, the methods and pharmaceutical compositions described herein include the use of N-oxides, crystalline forms (also known as polymorphs), as well as active metabolites of these compounds having the same type of activity. All tautomers of the compounds described herein are included within the scope of the compounds presented herein. Additionally, the compounds described herein encompass un-solvated as well as solvated forms with pharmaceutically acceptable solvents such as water, ethanol, and the like. The solvated forms of the compounds presented herein are also considered to be disclosed herein. In addition, the pharmaceutical compositions optionally include other medicinal or pharmaceutical agents, carriers, adjuvants, such as preserving, stabilizing, wetting or emulsifying agents, solution promoters, salts for regulating the osmotic pressure, buffers, and/or other therapeutically valuable substances.

Methods for the preparation of compositions comprising the compounds described herein include formulating the compounds with one or more inert, pharmaceutically acceptable excipients or carriers to form a solid, semi-solid or liquid. Solid compositions include, but are not limited to, powders, tablets, dispersible granules, capsules, cachets, and suppositories. Liquid compositions include solutions in which a compound is dissolved, emulsions comprising a compound, or a solution containing liposomes, micelles, or nanoparticles comprising a compound as disclosed herein. Semi-solid compositions include, but are not limited to, gels, suspensions and creams. The form of the pharmaceutical compositions described herein include liquid solutions or suspensions, solid forms suitable for solution or suspension in a liquid prior to use, or as emulsions. These compositions also optionally contain minor amounts of nontoxic, auxiliary substances, such as wetting or emulsifying agents, pH buffering agents, and so forth.

In some embodiments, pharmaceutical compositions comprising one or more compounds selected from compounds of Structure (I) illustratively takes the form of a liquid where the agents are present in solution, in suspension or both. Typically when the composition is administered as a suspension, a first portion of the agent is present in solution and a second portion of the agent is present in particulate form, in suspension in a liquid matrix. In some embodiments, a liquid composition includes a gel formulation. In other embodiments, the liquid composition is aqueous.

In certain embodiments, aqueous suspensions contain one or more polymers as suspending agents. Polymers include water-soluble polymers such as cellulosic polymers, e.g., hydroxypropyl methylcellulose, and water-insoluble polymers such as cross-linked carboxyl-containing polymers. Certain pharmaceutical compositions described herein comprise a mucoadhesive polymer, selected for example from carboxymethylcellulose, carbomer (acrylic acid polymer), poly(methylmethacrylate), polyacrylamide, polycarbophil, acrylic acid/butyl acrylate copolymer, sodium alginate and dextran.

Pharmaceutical compositions also, optionally, include solubilizing agents to aid in the solubility of one or more compounds selected from compounds of Structure (I). The term “solubilizing agent” generally includes agents that result in formation of a micellar solution or a true solution of the agent. Certain acceptable nonionic surfactants, for example polysorbate 80, are useful as solubilizing agents, as can ophthalmically acceptable glycols, polyglycols, e.g., polyethylene glycol 400, and glycol ethers.

Furthermore, pharmaceutical compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Compositions also, optionally, include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other pharmaceutical compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

Compositions may include one or more surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

Compositions may include one or more antioxidants to enhance chemical stability where required. Suitable antioxidants include, by way of example only, ascorbic acid and sodium metabisulfite.

In certain embodiments, aqueous suspension compositions are packaged in single-dose non-re-closable containers. Alternatively, multiple-dose re-closable containers are used, in which case it is typical to include a preservative in the composition.

In alternative embodiments, other delivery systems for hydrophobic pharmaceutical compounds are employed. Liposomes and emulsions are examples of delivery vehicles or carriers useful herein. In certain embodiments, organic solvents such as N-methylpyrrolidone are also employed. In additional embodiments, the compounds described herein are delivered using a sustained-release system, such as semipermeable matrices of solid hydrophobic polymers containing the therapeutic agent. Various sustained-release materials are useful herein. In some embodiments, sustained-release capsules release the compounds for a few weeks up to over 100 days. Depending on the chemical nature and the biological stability of the therapeutic reagent, additional strategies for protein stabilization are employed.

In certain embodiments, the formulations described herein comprise one or more antioxidants, metal chelating agents, thiol containing compounds and/or other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (1) pentosan polysulfate and other heparinoids, (m) divalent cations such as magnesium and zinc; or (n) combinations thereof.

In some embodiments, the concentration of one or more compounds selected from compounds of Structure (I) provided in the pharmaceutical compositions of the present disclosure is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v.

In some embodiments, the concentration of one or more compounds selected from compounds of Structure (I) provided in the pharmaceutical compositions of the present disclosure is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v.

In some embodiments, the amount the one or more compounds selected from compounds of Structure (I) provided in the pharmaceutical compositions of the present disclosure is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of the one or more compounds selected from compounds of Structure (I) provided in the pharmaceutical compositions of the present disclosure is in the range of 0.0001-10 g, 0.0005-9 g, 0.001-8 g, 0.005-7 g, 0.01-6 g, 0.05-5 g, 0.1-4 g, 0.5-4 g, or 1-3 g.

Packaging materials for use in packaging pharmaceutical compositions described herein include those found in, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. For example, the container(s) includes one or more compounds described herein, optionally in a composition or in combination with another agent as disclosed herein. The container(s) optionally have a sterile access port (for example the container is an intravenous solution bag or a vial having a stopper pierceable by a hypodermic injection needle). Such kits optionally comprise a compound with an identifying description or label or instructions relating to its use in the methods described herein.

For example, a kit typically includes one or more additional containers, each with one or more of various materials (such as reagents, optionally in concentrated form, and/or devices) desirable from a commercial and user standpoint for use of a compound described herein. Non-limiting examples of such materials include, but not limited to, buffers, diluents, filters, needles, syringes; carrier, package, container, vial and/or tube labels listing contents and/or instructions for use, and package inserts with instructions for use. A set of instructions will also typically be included. A label is optionally on or associated with the container. For example, a label is on a container when letters, numbers or other characters forming the label are attached, molded or etched into the container itself, a label is associated with a container when it is present within a receptacle or carrier that also holds the container, e.g., as a package insert. In addition, a label is used to indicate that the contents are to be used for a specific therapeutic application. In addition, the label indicates directions for use of the contents, such as in the methods described herein. In certain embodiments, the pharmaceutical compositions are presented in a pack or dispenser device which contains one or more unit dosage forms containing a compound provided herein. The pack for example contains metal or plastic foil, such as a blister pack. Or, the pack or dispenser device is accompanied by instructions for administration. Or, the pack or dispenser is accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In some embodiments, compositions containing a compound provided herein formulated in a compatible pharmaceutical carrier are prepared, placed in an appropriate container, and labeled for treatment of an indicated condition.

### Methods

Embodiments of the present disclosure are useful as modulators of NEK7 in a host species and thereby treatment of particular diseases (e.g., cancer).

The host or patient can belong to any mammalian species, for example a primate species, particularly humans; rodents, including mice, rats and hamsters; rabbits; horses, cows, dogs, cats, etc. Animal models are of interest for experimental investigations, providing a model for treatment of human disease.

In one embodiment, the present disclosure is useful as an inhibitor of NEK7. Therefore, the compounds of Structure (I) are also useful in the treatment of conditions resulting from that activation in a host species.

In another embodiment, the compounds of Structure (I) are useful as inhibitors of NEK7.

Embodiments of the disclosure also relate to the use of compounds according to Structure (I) and/or physiologically acceptable salts thereof for the prophylactic or therapeutic treatment and/or monitoring of diseases that are caused, mediated and/or modulated by NEK7 activity. Furthermore, embodiments of the disclosure relate to the use of compounds according to Structure (I) and/or physiologically acceptable salts thereof for the production of a medicament for the prophylactic or therapeutic treatment and/or monitoring of diseases that are caused, mediated and/or modulated by NEK7 activity. In certain embodiments, the disclosure provides the use of a compound according to Structure I or physiologically acceptable salts thereof, for the production of a medicament for the prophylactic or therapeutic treatment of a —NEK7 disorder.

In another embodiment, the present disclosure relates to a method of treating neoplastic diseases or conditions mediated by NEK7 by administering to a patient in need thereof a therapeutically effective amount of the compound of Structure (I).

In certain embodiments, the disease that can be treated with a compound of Structure (I) is a neoplastic disease (e.g., sarcomas, melanomas, carcinomas, leukemias, solid tumors). In some embodiments, the neoplastic disease is a cancer. In certain specific embodiments, the cancer is a sarcoma, melanoma, carcinoma, solid tumor, or combination thereof. In some embodiments, the cancer is a sarcoma. In some more specific embodiments, the cancer is a melanoma. In some embodiments, the cancer is a carcinoma. In some embodiments, the cancer is a solid tumor.

In certain other embodiments, the compounds of Structure (I) are used in methods for treatment of disorders or diseases selected from auto-immune, inflammatory disorders, cardiovascular diseases, neurodegenerative disorders, bacterial and viral infections, allergy, asthma, pancreatitis, multi-organ failure, kidney diseases, platelet aggregation, cancer, transplantation, sperm motility, erythrocyte deficiency, graft rejection, lung injuries, respiratory diseases, ischemic conditions, cancer, and combinations thereof.

In some embodiments, the disorders associated with NEK7 which are treatable with a compound of Structure (I) are selected from rheumatoid arthritis, psoriatic arthritis, osteoarthritis, systemic lupus erythematosus, lupus nephritis, ankylosing spondylitis, osteoporosis, systemic sclerosis, multiple sclerosis, psoriasis, type I diabetes, type II diabetes, inflammatory bowel disease (Crohn's Disease and ulcerative colitis), hyperimmunoglobulinemia D and periodic fever syndrome, cryopyrin associated periodic syndromes, Schnitzler's syndrome, systemic juvenile idiopathic arthritis, adult's onset Still's disease, gout, pseudogout, SAPHO syndrome, Castleman's disease, sepsis, stroke, atherosclerosis, celiac disease, DIRA (Deficiency of IL-1 Receptor Antagonist), Alzheimer's disease, Parkinson's disease, cancer, and combinations thereof.

In some embodiments, the disease or disorder is an NLRP3-mediated disorder. In certain embodiments, the disorder is selected from auto-immune, inflammatory disorders, cardiovascular diseases, neurodegenerative disorders, bacterial and viral infections, allergy, asthma, pancreatitis, multi-organ failure, kidney diseases, platelet aggregation, cancer, transplantation, sperm motility, erythrocyte deficiency, graft rejection, lung injuries, respiratory diseases, ischemic conditions, and combinations thereof.

In some embodiments, the disorder is selected from type II diabetes, atherosclerosis, Alzheimer's disease, aging, fatty liver, metabolic syndrome, asthma, psoriasis, obesity, acute and chronic tissue damage caused by infection, gout, arthritis, macular degeneration, enteritis, hepatitis, peritonitis, silicosis, UV-induced skin sunburn, contact hypersensitivity, sepsis, cancer, neurodegenerative disease, multiple sclerosis, and combinations thereof.

In some embodiments, the solid tumors that can be treated by administering compounds of the present disclosure include pancreatic cancer; bladder cancer; colorectal cancer; breast cancer, including metastatic breast cancer; prostate cancer, including androgen-dependent and androgen-independent prostate cancer; renal cancer, including, e.g., metastatic renal cell carcinoma; hepatocellular cancer; lung cancer, including, e.g., non-small cell lung cancer (NSCLC), bronchioloalveolar carcinoma (BAC), and adenocarcinoma of the lung; ovarian 30 cancer, including, e.g., progressive epithelial or primary peritoneal cancer; cervical cancer; gastric cancer; esophageal cancer; head and neck cancer, including, e.g., squamous cell carcinoma of the head and neck; melanoma; neuroendocrine cancer, including metastatic neuroendocrine tumors; brain tumors, including, e.g., glioma, anaplastic oligodendroglioma, adult glioblastoma multiforme, and adult anaplastic astrocytoma; bone cancer; and soft tissue sarcoma.

In some other embodiments, the cancer is a hematologic malignancy. Non-limiting examples of hematologic malignancy include acute myeloid leukemia (AML); chronic myelogenous leukemia (CMIL), including accelerated CML and CML blast phase (CML-BP); acute lymphoblastic leukemia (ALL); chronic lymphocytic leukemia (CLL); Hodgkin's disease (HD); non-Hodgkin's lymphoma (NHL), including follicular lymphoma and mantle cell lymphoma; B-cell lymphoma; T-cell lymphoma; multiple myeloma (MN); Waldenstrom's macroglobulinemia; myelodysplastic syndromes (MDS), including refractory anemia (RA), refractory anemia with ringed siderblasts (RARS), (refractory anemia with excess blasts (RAEB), and RAEB in transformation (RAEB-T); and myeloproliferative syndromes.

In some embodiments, the leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

In some embodiments, the cancer is selected from the group consisting of colorectal cancer, ovarian cancer, breast cancer, gastric cancer, prostate cancer, brain cancer, bone cancer, bladder cancer, head and neck cancer, lung cancer, renal cancer, pancreatic cancer, sarcoma, leukemia, and lymphoma.

In some specific embodiments, the sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

In certain embodiments, the melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

In some embodiments, the carcinoma that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

Also included herein are methods of treatment in which at least one compound of Structure (I) is administered in combination with an anti-inflammatory or a therapeutic agent. Anti-inflammatory agents include but are not limited to NSAIDs, non-specific and COX-2 specific cyclooxygenase enzyme inhibitors, gold compounds, corticosteroids, methotrexate, tumor necrosis factor (TNF) antagonists, immunosuppressants and methotrexate. Examples of NSAIDs include, but are not limited to, ibuprofen, flurbiprofen, naproxen and naproxen sodium, diclofenac, combinations of diclofenac sodium and misoprostol, sulindac, oxaprozin, diflunisal, piroxicam, indomethacin, etodolac, fenoprofen calcium, ketoprofen, sodium nabumetone, sulfasalazine, tolmetin sodium, hydroxychloroquine, and combinations thereof.

Examples of NSAIDs also include COX-2 specific inhibitors such as celecoxib, valdecoxib, lumiracoxib, and/or etoricoxib.

In some embodiments, the anti-inflammatory agent is a salicylate. Salicylates include by are not limited to acetylsalicylic acid or aspirin, sodium salicylate, and choline and magnesium salicylates.

The anti-inflammatory agent may also be a corticosteroid. For example, the corticosteroid may be cortisone, dexamethasone, methylprednisolone, prednisolone, prednisolone sodium phosphate, or prednisone.

In additional embodiments the anti-inflammatory agent is a gold compound such as gold sodium thiomalate or auranofin.

The disclosure also includes embodiments in which the anti-inflammatory agent is a metabolic inhibitor such as a dihydrofolate reductase inhibitor, such as methotrexate or a dihydroorotate dehydrogenase inhibitor, such as leflunomide.

Therapeutic agents can also include agents for pain and inflammation such as histamine and histamine antagonists, bradykinin and bradykinin antagonists, 5-hydroxytryptamine (serotonin), lipid substances that are generated by biotransformation of the products of the selective hydrolysis of membrane phospholipids, eicosanoids, prostaglandins, thromboxanes, leukotrienes, aspirin, nonsteroidal anti-inflammatory agents, analgesic-antipyretic agents, agents that inhibit the synthesis of prostaglandins and thromboxanes, selective inhibitors of the inducible cyclooxygenase, selective inhibitors of the inducible cyclooxygenase-2, autacoids, paracrine hormones, somatostatin, gastrin, cytokines that mediate interactions involved in humoral and cellular immune responses, lipid-derived autacoids, eicosanoids, β-adrenergic agonists, ipratropium, glucocorticoids, methylxanthines, sodium channel blockers, opioid receptor agonists, calcium channel blockers, membrane stabilizers and leukotriene inhibitors.

Still other embodiments of the disclosure pertain to combinations in which at least one active agent is an immunosuppressant compound such as an immunosuppressant compound chosen from methotrexate, leflunomide, cyclosporine, tacrolimus, azathioprine, and mycophenolate mofetil.

The disclosed compounds of Structure (I) can be administered in combination with other known therapeutic agents, including anticancer agents. As used here, the term “anticancer agent” relates to any agent which is administered to a patient with cancer for the purposes of treating the cancer.

In some embodiments the anti-cancer agents belong to the following categories
Alkylating agents: such as altretamine, bendamustine, busulfan, carmustine, chlorambucil, chlormethine, cyclophosphamide, dacarbazine, ifosfamide, improsulfan, tosilate, lomustine, melphalan, mitobronitol, mitolactol, nimustine, ranimustine, temozolomide, thiotepa, treosulfan, mechloretamine, carboquone; apaziquone, fotemustine, glufosfamide, palifosfamide, pipobroman, trofosfamide, uramustine, TH-3024, VAL-0834;
Platinum Compounds: such as carboplatin, cisplatin, eptaplatin, miriplatine hydrate, oxaliplatin, lobaplatin, nedaplatin, picoplatin, satraplatin; lobaplatin, nedaplatin, picoplatin, satraplatin;
DNA altering agents: such as amrubicin, bisantrene, decitabine, mitoxantrone, procarbazine, trabectedin, clofarabine; amsacrine, brostallicin, pixantrone, laromustine; Topoisomerase Inhibitors: such as etoposide, irinotecan, razoxane, sobuzoxane, teniposide, topotecan; amonafide, belotecan, elliptinium acetate, voreloxin; Microtubule modifiers: such as cabazitaxel, docetaxel, eribulin, ixabepilone, paclitaxel, vinblastine, vincristine, vinorelbine, vindesine, vinflunine; fosbretabulin, tesetaxel;
Antimetabolites: such as asparaginase3, azacitidine, calcium levofolinate, capecitabine, cladribine, cytarabine, enocitabine, floxuridine, fludarabine, fluorouracil, gemcitabine, mercaptopurine, methotrexate, nelarabine, pemetrexed, pralatrexate, azathioprine, thioguanine, carmofur; doxifluridine, elacytarabine, raltitrexed, sapacitabine, tegafur, trimetrexate;
Anticancer antibiotics: such as bleomycin, dactinomycin, doxorubicin, epirubicin, idarubicin, levamisole, miltefosine, mitomycin C, romidepsin, streptozocin, valrubicin, zinostatin, zorubicin, daunurobicin, plicamycin; aclarubicin, peplomycin, pirarubicin;
Hormones/Antagonists: such as abarelix, abiraterone, bicalutamide, buserelin, calusterone, chlorotrianisene, degarelix, dexamethasone, estradiol, fluocortolone fluoxymesterone, flutamide, fulvestrant, goserelin, histrelin, leuprorelin, megestrol, mitotane, nafarelin, nandrolone, nilutamide, octreotide, prednisolone, raloxifene, tamoxifen, thyrotropin alfa, toremifene, trilostane, triptorelin, diethylstilbestrol; acolbifene, danazol, deslorelin, epitiostanol, orteronel, enzalutamide;
Aromatase inhibitors: such as aminoglutethimide, anastrozole, exemestane, fadrozole, letrozole, testolactone; formestane;
Small molecule kinase inhibitors: such as crizotinib, dasatinib, erlotinib, imatinib, lapatinib, nilotinib, pazopanib, regorafenib, ruxolitinib, sorafenib, sunitinib, vandetanib, vemurafenib, bosutinib, gefitinib, axitinib; afatinib, alisertib, dabrafenib, dacomitinib, dinaciclib, dovitinib, enzastaurin, nintedanib, lenvatinib, linifanib, linsitinib, masitinib, midostaurin, motesanib, neratinib, orantinib, perifosine, ponatinib, radotinib, rigosertib, tipifamib, tivantinib, tivozanib, trametinib, pimasertib, brivanib alaninate, cediranib.

In some embodiments, medicaments which are administered in conjunction with the compounds described herein include any suitable drugs usefully delivered by inhalation for example, analgesics, e.g. codeine, dihydromorphine, ergotamine, fentanyl or morphine; anginal preparations, e.g. diltiazem; antiallergics, e.g. cromoglycate, ketotifen or nedocromil; anti-infectives, e.g. cephalosporins, penicillins, streptomycin, sulphonamides, tetracyclines or pentamidine; antihistamines, e.g. methapyrilene; anti-inflammatories, e.g. beclomethasone, flunisolide, budesonide, tipredane, triamcinolone acetonide or fluticasone; antitussives, e.g. noscapine; bronchodilators, e.g. ephedrine, adrenaline, fenoterol, formoterol, isoprenaline, metaproterenol, phenylephrine, phenylpropanolamine, pirbuterol, reproterol, rimiterol, salbutamol, salmeterol, terbutalin, isoetharine, tulobuterol, orciprenaline or (−)-4-amino-3,5-dichloro-α-[[[6-[2-(2-pyridinyl)ethoxy]hexyl]-amino]methyl]benzenemethanol; diuretics, e.g., amiloride; anticholinergics, e.g., ipratropium, atropine or oxitropium; hormones, e.g., cortisone, hydrocortisone or prednisolone; xanthines, e.g., aminophylline, choline theophyllinate, lysine theophyllinate or theophylline; and therapeutic proteins and peptides, e.g., insulin or glucagon. It will be clear to a person skilled in the art that, where appropriate, the medicaments are used in the form of salts (e.g., as alkali metal or amine salts or as acid addition salts) or as esters (e.g., lower alkyl esters) or as solvates (e.g., hydrates) to optimize the activity and/or stability of the medicament.

The agents disclosed herein or other suitable agents are administered depending on the condition being treated. Hence, in some embodiments the one or more compounds of the disclosure will be co-administered with other agents as described above. When used in combination therapy, the compounds described herein are administered with the second agent simultaneously or separately. This administration in combination can include simultaneous administration of the two agents in the same dosage form, simultaneous administration in separate dosage forms, and separate administration. That is, a compound described herein and any of the agents described above can be formulated together in the same dosage form and administered simultaneously. Alternatively, a compound of the disclosure and any of the agents described above can be simultaneously administered, wherein both the agents are present in separate formulations. In another alternative, a compound of the present disclosure can be administered just followed by and any of the agents described above, or vice versa. In some embodiments of the separate administration protocol, a compound of the disclosure and any of the agents described above are administered a few minutes apart, or a few hours apart, or a few days apart.

In some embodiments, the compounds of Structure (I) are administered as a mono-therapy.

For identification of a signal transduction or a mechanistic pathway and for detection of interactions between various signal transduction pathways, various scientists have developed suitable models or model systems, for example cell culture models and models of transgenic animals. For the determination of certain stages in the signal transduction cascade, interacting compounds can be utilized in order to modulate the signal. The compounds of embodiments of the disclosure can also be used as reagents for testing NEK7-dependent signal transduction pathways in animals and/or cell culture models or in the clinical diseases mentioned in this application.

The methods of embodiments of embodiments of the disclosure can be performed either in vitro or in vivo. The susceptibility of a particular cell to treatment with the compounds of Structure (I) can be particularly determined by in vitro tests, whether in the course of research or clinical application. Typically, a culture of the cell is combined with a compound at various concentrations for a period of time which is sufficient to allow the active agents to inhibit NEK7 activity, usually between about one hour and one week. In-vitro treatment can be carried out using cultivated cells from a biopsy sample or cell line.

In some embodiments, the IC₅₀ of the compounds of Structure (I) to inhibit NEK7 was determined by the concentration of the compound required to inhibit 50% of the activity of the NEK kinase. The compounds of Structure (I) exhibited potency values of IC₅₀ of less than about 5 mM, preferably less than about 1 mM and even more preferably less than about 0.100 mM as described in further detail in the Examples.

The examples and preparations provided below further illustrate and exemplify the compounds of the present disclosure and methods of preparing and testing such compounds. It is to be understood that the scope of the present disclosure is not limited in any way by the scope of the following examples and preparations. In the following examples, and throughout the specification and claims, molecules with a single stereocenter, unless otherwise noted, exist as a racemic mixture. Those molecules with two or more stereocenters, unless otherwise noted, exist as a racemic mixture of diastereomers. Single enantiomers/diastereomers may be obtained by methods known to those skilled in the art.

Methods for producing the compounds described herein is provided below. In general, starting components may be obtained from sources such as Sigma Aldrich, Lancaster Synthesis, Inc., Maybridge, Matrix Scientific, TCI, and Fluorochem USA, etc. or synthesized according to sources known to those skilled in the art (see, for example, Advanced Organic Chemistry: Reactions, Mechanisms, and Structure, 5th edition (Wiley, December 2000)) or prepared as described herein.

The following General Reaction Schemes illustrate exemplary methods for preparation of compounds of Structure (I):

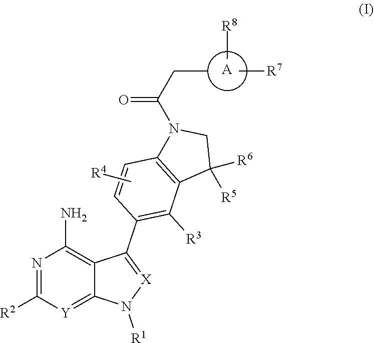

or pharmaceutically acceptable salts, stereoisomers or prodrug thereof, wherein each of A, X, Y, R¹, R², R³, R⁴, R⁵, R⁶, R⁷, and R⁸ are as defined herein.

### General Reaction Scheme 1

The following General Reaction Scheme, wherein X¹ is halogen, and A, X, Y, R¹, R², R³, R⁴, R⁵, R⁶, R⁷, and R⁸ have the meanings described herein, illustrate examples of methods of making the compounds of Structure (I):

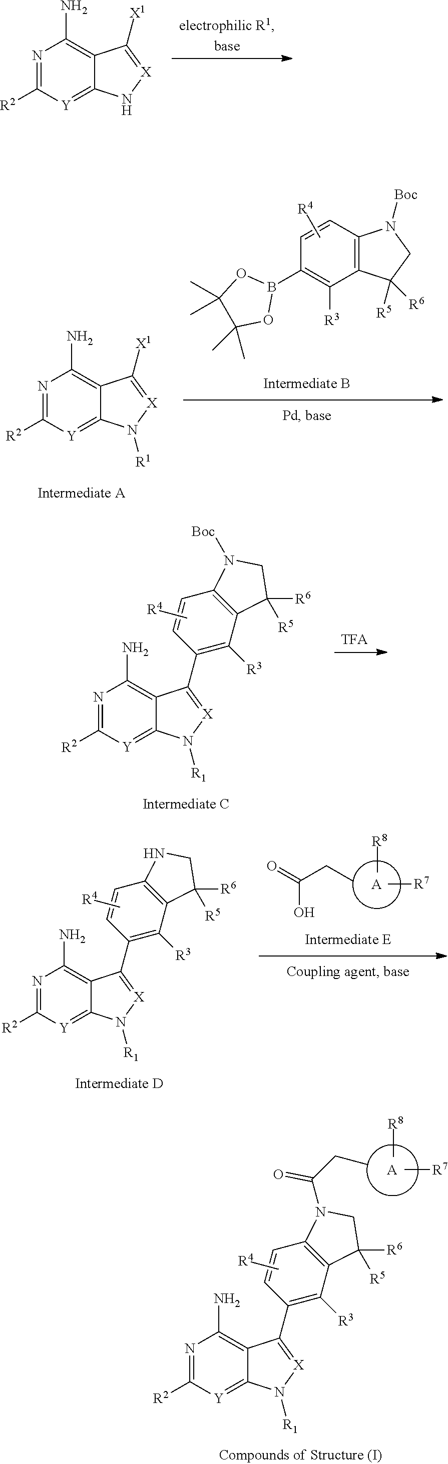

As shown in General Reaction Scheme 1, alkylation of the pyrrole or pyrazole of the bicyclic ring system with an appropriate electrophile in the presence of a base affords Intermediate A. Subsequent palladium-catalyzed arylation using boronate Intermediate B leads to carbamate Intermediate C which is de-protected with trifluoroacetic acid to yield amine Intermediate D. This is in turn reacted with carboxylic acid Intermediate E in the presence of an appropriate coupling agent and base to afford compounds of Structure (I).

Any of the above reaction schemes can be modified at any step to add and/or modify a substituent or change the order of the steps as appropriate during any stage of the overall synthesis of desired compounds.

It will also be appreciated by those skilled in the art that in the processes for preparing the compounds described herein the functional groups of intermediate compounds may need to be protected by suitable protecting groups. Such functional groups include, but are not limited to, hydroxy, amino, mercapto and carboxylic acid. Suitable protecting groups for hydroxy include trialkylsilyl or diarylalkylsilyl (for example, t-butyldimethylsilyl, t-butyldiphenylsilyl or trimethylsilyl), tetrahydropyranyl, benzyl, and the like. Suitable protecting groups for amino, amidino and guanidino include t-butoxycarbonyl, benzyloxycarbonyl, and the like. Suitable protecting groups for mercapto include —C(O)—R″ (where R″ is alkyl, aryl or arylalkyl), p-methoxybenzyl, trityl and the like. Suitable protecting groups for carboxylic acid include alkyl, aryl or arylalkyl esters. Protecting groups are optionally added or removed in accordance with standard techniques, which are known to one skilled in the art and as described herein. The use of protecting groups is described in detail in Green, T. W. and P. G. M. Wutz, Protective Groups in Organic Synthesis (1999), 3rd Ed., Wiley. As one of skill in the art would appreciate, the protecting group may also be a polymer resin such as a Wang resin, Rink resin or a 2-chlorotrityl-chloride resin.

It will also be appreciated by those skilled in the art, although such protected derivatives of compounds of this disclosure may not possess pharmacological activity as such, they may be administered to a mammal and thereafter metabolized in the body to form compounds of the disclosure which are pharmacologically active. Such derivatives may therefore be described as “prodrugs.” Prodrugs of compounds of this disclosure are included within the scope of embodiments of the disclosure.

### EXAMPLES

The following examples are provided for exemplary purposes.

### General Procedures:

All proton NMR experiments were recorded on a Bruker NEO Spectrometer equipped with a BBFO probe at 400 MHz. Deuterated solvents contained less than 0.05% v/v tetramethylsilane which was used as the reference signal (set at 0.00 ppm). When deuterated solvents did not contain tetramethylsilane, the residual nondeuterated solvent peaks were used as a reference signal, as per published guidelines (J. Org. Chem. 1997, 62(21), 7512-7515). Chemical shifts are expressed in parts per million (ppm, δ units). Coupling constants are in hertz (Hz). Splitting patterns describe apparent multiplicities and are designated as s (singlet), d (doublet), t (triplet), q (quartet), m (multiplet), qt (quintuplet) or brs (broad singlet).

LC/MS analyses were performed on an Agilent Technologies UHPLC 1290 Infinity II with a G6125 MS detector.

Microwave reactions were conducted with a Monowave 300 by Anton Paar GmbH using standard protocols.

### Abbreviations:

° C. (degree Celsius); ¹H NMR (proton Nuclear Magnetic Resonance); ACN (acetonitrile); AIBN (azobisisobutyronitrile); B₂pin₂ (bis(pinacolato)diboron); bipy (2,2′-bipyridine); Boc₂O (di-tert-butyl dicarbonate); Cu(OAC)₂ (copper (II) acetate); DCE (dichloroethane); DCM (dichloromethane); DIPEA (N,N-diisopropylethylamine); DMAP (4-dimethylaminopyridine); DMF (N,N-dimethylformamide); DMSO-d₆ (deuterated dimethylsulfoxide); eq (equivalent); EtOAc (ethyl acetate); g (gram); h (hour); HATU (1-[bis(dimethylamino)methylene]-1H-1,2,3-triazolo[4,5-b]pyridinium 3-oxide hexafluorophosphate); HPLC (High Performance Liquid Chromatography); LC-MS (Liquid Chromatography Mass Spectrometry); MeOH (methanol); mg (milligram); min (minute); mL (milliliter); mmol (millimole); NBS (N-bromosuccinimide); NIS (N-iodosuccinimide); Pd(PPh₃)₄ (palladium-tetrakis(triphenylphosphine)); PdCl₂(dppf) ([1,1′-bis(diphenylphosphino)ferrocene]palladium(II) dichloride); TBTU (N,N,N′,N′-tetramethyl-O-(benzotriazol-1-yl)uronium tetrafluoroborate); TFA (trifluoroacetic acid); THE (tetrahydrofuran); TLC (Thin Layer Chromatography)

### Synthetic Examples (Compound Intermediates)

### Intermediate A1

### 1-CYCLOPROPYL-3-IODO-1H-PYRAZOLO[3,4-D]PYRIMIDIN-4-AMINE

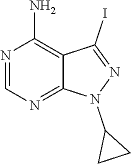 [SMILES: Nc1ncnc2c1c(I)nn2C1CC1]

Cu(OAc)₂ (0.348 g, 1.916 mmol), bipy (0.299 g, 1.916 mmol), and NaHCO₃ (0.322 g, 3.830 mmol) were added to a solution of 3-iodo-1H-pyrazolo[3,4-d]pyrimidin-4-amine (0.500 g, 1.916 mmol) and cyclopropylboronic acid (0.329 g, 3.830 mmol) in dichloroethane (10 mL). The resulting mixture was stirred at 70° C. under oxygen atmosphere for 12 h. Following completion of the reaction (as indicated by TLC), the reaction mixture was filtered through a pad of Celite® (i.e., diatomaceous earth) which was then rinsed with DCM (2×20 mL). The combined filtrates were washed with water (20 mL) and brine (25 mL), dried over Na₂SO₄, filtered, and concentrated under reduced pressure to yield crude material which was purified by flash chromatography (silica gel 230-400 mesh, eluting with 20% EtOAc in petroleum ether), giving the title compound as an off-white solid (0.24 g, 36% yield). ¹H NMR (400 MHz, DMSO-d₆) δ=8.21 (s, 1H), 3.74-3.79 (m, 1H), 1.11-1.15 (m, 2H), 1.04-1.09 (m, 2H); LCMS: 301.8 [M+H].

### Intermediate A2

### 3-IODO-1-ISOPROPYL-1H-PYRAZOLO[3,4-D]PYRIMIDIN-4-AMINE

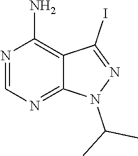

Cs₂CO₃ (12.38 g, 38.31 mmol) and 2-iodopropane (3.60 g, 21.16 mmol) were added to a solution of 3-iodo-1H-pyrazolo[3,4-d]pyrimidin-4-amine (5.00 g, 19.15 mmol) in DMF (25 mL) in a sealed tube. The reaction mixture was stirred at 90° C. for 16 h and, following completion of the reaction (as indicated by TLC), was poured into crushed ice (50 g) and stirred for 15 min. The resulting solid was filtered, washed with water (2×5 mL), and dried to afford the title compound as an off-white solid (3.25 g, 56% yield). ¹H NMR (400 MHz, DMSO-d₆) δ=8.18 (s, 1H), 4.93-4.99 (m, 1H), 1.42 (d, J 6.8 Hz, 6H); LCMS: 303.8 [M+H].

### Intermediate A3

### 3-IODO-1-(OXETAN-3-YL)-1H-PYRAZOLO[3,4-D]PYRIMIDIN-4-AMINE

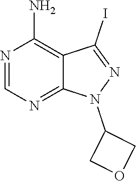 [SMILES: Nc1ncnc2c1c(I)nn2C1COC1]

The title compound was prepared via a similar procedure described for Intermediate A2, using 3-iodooxetane as the electrophile. ¹H NMR (400 MHz, DMSO-d₆) δ=8.21 (s, 1H), 5.89-5.93 (m, 1H), 4.93-5.00 (m, 4H); LCMS: 318.0 [M+H].

### Intermediate A4

### 3-IODO-1-(TETRAHYDRO-2H-PYRAN-4-YL)-1H-PYRAZOLO[3,4-D]PYRIMIDIN-4-AMINE

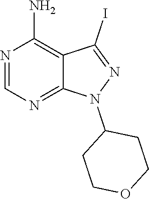 [SMILES: Nc1ncnc2c1c(I)nn2C1CCOCC1]

The title compound was prepared via a similar procedure described for Intermediate A2, using tetrahydro-2H-pyran-4-yl 4-methylbenzenesulfonate as the electrophile. ¹H NMR (400 MHz, DMSO-d₆) δ=8.20 (s, 1H), 4.82-4.89 (m, 1H), 3.96-4.00 (m, 2H), 3.48-3.54 (m, 2H), 2.05-2.16 (m, 2H), 1.82-1.86 (m, 2H); LCMS: 345.9 [M+H].

### Intermediate A5

### 7-CYCLOPROPYL-5-IODO-7H-PYRROLO[2,3-D]PYRIMIDIN-4-AMINE

### Step 1: Synthesis of 4-chloro-5-iodo-7H-pyrrolo[2,3-d]pyrimidine

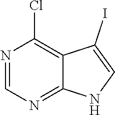 [SMILES: Clc1ncnc2[nH]cc(I)c12]

NIS(1.465 g, 6.51 mmol) was added to a solution of 4-chloro-7H-pyrrolo[2,3-d]pyrimidine (1.000 g, 6.51 mmol) in DMF (10 mL) and the resulting solution was stirred at 25° C. for 12 h. Following completion of the reaction (as indicated by TLC), the reaction mixture was poured into ice cold water (100 mL) and stirred for 15 min. The resulting precipitate was filtered, washed with water (2×25 mL), and dried to afford the title compound as an off-white solid (1.7 g, 93% yield). ¹H NMR (400 MHz, DMSO-d₆) δ=12.96 (bs, 1H), 8.60 (s, 1H), 7.95 (d, J 2.4 Hz, 1H); LCMS: 279.9 [M+H].

### Step 2: Synthesis of 4-chloro-7-cyclopropyl-5-iodo-7H-pyrrolo[2,3-d]pyrimidine

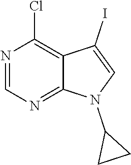 [SMILES: Clc1ncnc2c1c(I)cn2C1CC1]

Cu(OAc)₂ (0.650 g, 3.58 mmol), bipy (0.559 g, 3.58 mmol), and NaHCO₃ (0.601 g, 7.16 mmol) were added to a solution of 4-chloro-5-iodo-7H-pyrrolo[2,3-d]pyrimidine (1.000 g, 3.58 mmol) and cyclopropylboronic acid (0.615 g, 7.16 mmol) in dry dichloroethane (10 mL). The resulting mixture was stirred under oxygen atmosphere at 70° C. for 12 h. Following completion of the reaction (as indicated by TLC), the reaction mixture was filtered through a pad of Celite® (i.e., diatomaceous earth) which was then rinsed with DCM (2×20 mL). The combined filtrates were washed with water (20 mL) and brine (25 mL), dried over Na₂SO₄, filtered, and concentrated under reduced pressure to yield crude material which was purified by flash chromatography (silica gel 230-400 mesh, eluting with 15% EtOAc in petroleum ether), giving the title compound as an off-white solid (0.7 g, 61% yield). ¹H NMR (400 MHz, DMSO-d₆) δ=8.67 (s, 1H), 7.96 (s, 1H), 3.63-3.69 (m, 1H), 1.06-1.10 (m, 4H); LCMS: 319.9 [M+H].

### Step 3: Synthesis of 7-cyclopropyl-5-iodo-7H-pyrrolo[2,3-d]pyrimidin-4-amine

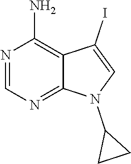 [SMILES: Nc1ncnc2c1c(I)cn2C1CC1]

A mixture of 4-chloro-7-cyclopropyl-5-iodo-7H-pyrrolo[2,3-d]pyrimidine (1.00 g, 2.191 mmol) and NH₄OH (25% in water, 5 mL) was subjected to microwave irradiation at 150° C. for 1 h. Following completion of the reaction (as indicated by TLC), the reaction mixture was concentrated under reduced pressure to yield the title compound as an off-white solid (0.75 g, 80% yield). ¹H NMR (400 MHz, DMSO-d₆) δ=8.12 (s, 1H), 7.39 (s, 1H), 6.57 (bs, 2H), 3.48-3.54 (m, 1H), 0.97-1.01 (m, 4H); LCMS: 301.0 [M+H].

### Intermediate A6

### 5-BROMO-7-ISOPROPYL-7H-PYRROLO[2,3-D]PYRIMIDIN-4-AMINE

### Step 1: Synthesis of 5-bromo-4-chloro-7H-pyrrolo[2,3-d]pyrimidine

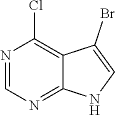 [SMILES: Clc1ncnc2[nH]cc(Br)c12]

The title compound was prepared via a similar procedure described for step 1 of Intermediate A5, starting from 4-chloro-7H-pyrrolo[2,3-d]pyrimidine (1.00 g, 6.51 mmol) and NBS (1.16 g, 6.51 mmol), and was obtained as a yellow solid (1.2 g, 80% yield). ¹H NMR (400 MHz, DMSO-d₆) δ=12.95 (bs, 1H), 8.63 (s, 1H), 7.96 (d, J 2.4 Hz, 1H); LCMS: 232.0 [M+H].

### Step 2: Synthesis of 5-bromo-4-chloro-7-isopropyl-7H-pyrrolo[2,3-d]pyrimidine

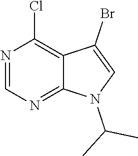

A mixture or 5-bromo-4-chloro-7H-pyrrolo[2,3-d]pyrimidine (0.50 g, 2.15 mmol), K₂CO₃ (0.59 g, 4.30 mmol), and 2-iodopropane (0.22 g, 1.30 mmol) in dry DMF (5 mL) was stirred at 90° C. for 16 h in a sealed tube. Following completion of the reaction (as indicated by TLC), the reaction mixture was poured into crushed ice (50 g) and stirred for 15 min. The resulting precipitate was filtered, washed with water (2×5 mL), and dried to afford the title compound as a white solid (0.3 g, 59% yield). ¹H NMR (400 MHz, DMSO-d₆) δ=8.67 (s, 1H), 8.20 (s, 1H), 5.06-5.09 (m, 1H), 1.48 (d, J 6.8 Hz, 6H); LCMS: 273.7 [M+H].

### Step 3: Synthesis of 5-bromo-7-isopropyl-7H-pyrrolo[2,3-d]pyrimidin-4-amine

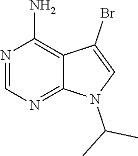

The title compound was prepared via a similar procedure described for step 3 of Intermediate A5, starting from 5-bromo-4-chloro-7-isopropyl-7H-pyrrolo[2,3-d]pyrimidine (0.3 g, 1.1 mmol) and NH₄OH (25% in water, 1.5 mL), and was obtained as an off-white solid (0.17 g, 61% yield). LCMS: 256.9 [M+H].

### Intermediate A7

### 2-(4-AMINO-5-IODO-7H-PYRROLO[2,3-D]PYRIMIDIN-7-YL)ETHAN-1-OL

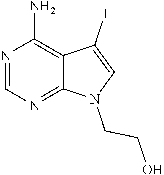 [SMILES: Nc1ncnc2c1c(I)cn2CCO]

The title compound was prepared as reported in PCT Publication No. WO 2021/226547 A2.

### Intermediate A8

### 2-(4-AMINO-3-IODO-1H-PYRAZOLO[3,4-D]PYRIMIDIN-1-YL)ETHAN-1-OL

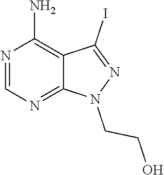 [SMILES: Nc1ncnc2c1c(I)nn2CCO]

The title compound was prepared as reported in PCT Publication No. WO 2011/119663 A1.

### Intermediate A9

### 5-IODO-7-TOSYL-7H-PYRROLO[2,3-D]PYRIMIDIN-4-AMINE

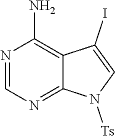 [SMILES: Cc1ccc(S(=O)(=O)n2cc(I)c3c(N)ncnc32)cc1]

The title compound was prepared as reported in J. Med. Chem. 2019, 62, 8, 4158-4173.

### Intermediate A10

### 1-(4-AMINO-5-IODO-7H-PYRROLO[2,3-D]PYRIMIDIN-7-YL)-2-METHYLPROPAN-2-OL

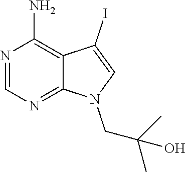 [SMILES: CC(C)(O)Cn1cc(I)c2c(N)ncnc21]

The title compound was prepared as reported in PCT Publication No. WO 2021/226547 A2.

### Intermediate A11

### 3-IODO-1-(2-METHOXYETHYL)-1H-PYRAZOLO[3,4-D]PYRIMIDIN-4-AMINE

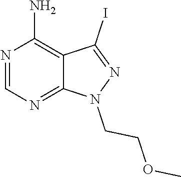

K₂CO₃ (0.397 g, 2.870 mmol) and 1-bromo-2-methoxyethane (0.319 g, 2.299 mmol) were added to a solution of 3-iodo-1H-pyrazolo[3,4-d]pyrimidin-4-amine (0.500 g, 1.916 mmol) in DMF (6 mL) and the resulting solution was stirred at 80° C. for 12 h in a sealed tube. Following completion of the reaction (as indicated by TLC), the reaction mixture was poured into crushed ice (25 g) and extracted with EtOAc (2×50 mL). The combined organic extracts were dried over Na₂SO₄, filtered, and concentrated under reduced pressure to give the title product (0.500 g, crude) which was used without further purification. ¹H NMR (400 MHz, DMSO-d₆) δ=8.20 (s, 1H), 4.43 (t, J 5.6 Hz, 2H), 3.75 (t, J 5.6 Hz, 2H), 3.20 (s, 3H); LCMS: 319.8 [M+H].

### Intermediate A12

### 5-IODO-7-(2-METHOXYETHYL)-7H-PYRROLO[2,3-D]PYRIMIDIN-4-AMINE

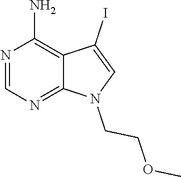

The title compound was prepared as reported in PCT Publication No. WO 2014/184069 A1.

### Intermediate A13

### 7-(3-(BENZYLOXY)CYCLOBUTYL)-5-IODO-7H-PYRROLO[2,3-D]PYRIMIDIN-4-AMINE

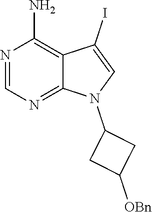 [SMILES: Nc1ncnc2c1c(I)cn2C1CC(OCc2ccccc2)C1]

The title compound was prepared as reported in PCT Publication No. WO 2021/226547 A2.

### Intermediate A14

### 5-IODO-7-(1-METHYLPIPERIDIN-4-YL)-7H-PYRROLO[2,3-D]PYRIMIDIN-4-AMINE

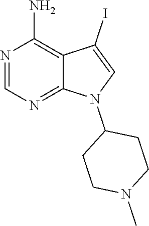 [SMILES: CN1CCC(n2cc(I)c3c(N)ncnc32)CC1]

The title compound was prepared as reported in PCT Publication No. WO 2017/220477 A1.

### Intermediate A15

### 1-CYCLOPROPYL-N-(2,4-DIMETHOXYBENZYL)-3-IODO-1H-PYRAZOLO[4,3-C]PYRIDIN-4-AMINE

### Step 1: Synthesis of 4-chloro-3-iodo-1H-pyrazolo[4,3-c]pyridine

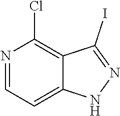 [SMILES: Clc1nccc2[nH]nc(I)c12]

KOH (1.320 g, 23.0 mmol) and iodine (1.620 g, 12.8 mmol) were added to a solution of 4-chloro-1H-pyrazolo[4,3-c]pyridine (1.000 g, 6.4 mmol) in dioxane (10 mL) and the resulting mixture was stirred at 75° C. for 4 h. Following completion of the reaction (as indicated by TLC), the reaction mixture was filtered over a pad of Celite® (i.e., diatomaceous earth). The filtrate was concentrated under reduced pressure to give crude material which was purified by reverse-phase column chromatography, giving the title compound as a white solid (0.633 g, 63% yield). ¹H NMR (400 MHz, DMSO-d₆) δ=14.12 (bs, 1H), 8.14 (d, J 6.0 Hz, 1H), 7.66 (d, J 5.6 Hz, 1H); LCMS: 279.9 [M+H].

### Step 2: Synthesis of 4-chloro-1-cyclopropyl-3-iodo-1H-pyrazolo[4,3-c]pyridine

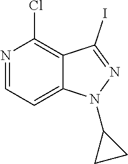 [SMILES: Clc1nccc2c1c(I)nn2C1CC1]

The title compound was prepared via a similar procedure described for intermediate A1, starting from 4-chloro-3-iodo-1H-pyrazolo[4,3-c]pyridine (0.630 g, 2.20 mmol) and cyclopropylboronic acid (0.329 g, 3.83 mmol), and was obtained as a white solid (0.430 g, 60% yield). ¹H NMR (400 MHz, DMSO-d₆) δ=8.21 (d, J 6.0 Hz, 1H), 7.81 (d, J 5.6 Hz, 1H), 3.84-3.89 (m, 1H), 1.14-1.17 (m, 4H); LCMS: 319.7 [M+H].

### Step 3: Synthesis of 1-cyclopropyl-N-(2,4-dimethoxybenzyl)-3-iodo-1H-pyrazolo[4,3-c]pyridin-4-amine

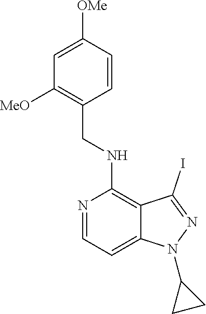 [SMILES: COc1ccc(CNc2nccc3c2c(I)nn3C2CC2)c(OC)c1]

A mixture of 4-chloro-1-cyclopropyl-3-iodo-1H-pyrazolo[4,3-c]pyridine (0.880 g, 2.75 mmol) and (2,5-dimethoxyphenyl)methanamine (1.245 mL, 8.26 mmol) in n-BuOH (10 mL) was stirred at 110° C. for 12 h. Following completion of the reaction (as indicated by LCMS), the reaction mixture was concentrated under reduced pressure to afford crude material which was purified by Isolera (silica gel 230-400 mesh, eluting with 40% EtOAc in petroleum ether) to yield the title product as yellow gum (1.0 g, 80% yield). ¹H NMR (400 MHz, DMSO-d₆) δ=7.81 (d, J 6.4 Hz, 1H), 7.19 (d, J 8.4 Hz, 1H), 6.87 (d, J 6.0 Hz, 1H), 6.60 (d, J 2.4 Hz, 1H), 6.53-6.56 (m, 1H), 6.45-6.47 (m, 1H), 4.62 (d, J 5.6 Hz, 2H), 3.88 (s, 3H), 3.73 (s, 3H), 3.66-3.70 (m, 1H), 1.01-1.10 (m, 4H); LCMS: 451.0 [M+H].

### Intermediate A16

### 5-IODO-7-(1-METHYLPIPERIDIN-3-YL)-7H-PYRROLO[2,3-D]PYRIMIDIN-4-AMINE

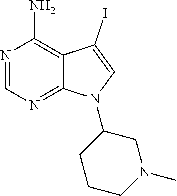 [SMILES: CN1CCCC(n2cc(I)c3c(N)ncnc32)C1]

The title compound was prepared as reported in PCT Publication No. WO 2014/184069 A1.

### Intermediate A17

### 5-IODO-7-(1-METHYFA18LAZETIDIN-3-YL)-7H-PYRROLO[2,3-D]PYRIMIDIN-4-AMINE

### Step 1: Synthesis of 7-(azetidin-3-yl)-5-iodo-7H-pyrrolo[2,3-d]pyrimidin-4-amine

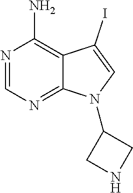 [SMILES: Nc1ncnc2c1c(I)cn2C1CNC1]

TFA (2.5 mL) was added to a solution of tert-butyl 3-(4-amino-5-iodo-7H-pyrrolo[2,3-d]pyrimidin-7-yl)azetidine-1-carboxylate (prepared as reported in PCT Publication No. WO 2017/215485 A1, 0.520 g, 1.252 mmol) in DCM (5 mL) and the resulting mixture was stirred at 25° C. for 12 h. Following completion of the reaction (as indicated by UPLC), the reaction mixture was concentrated under reduced pressure to afford the title compound (0.4 g, crude) which was used without further purification. LCMS: 316.0 [M+H].

### Step 2: Synthesis of 5-iodo-7-(1-methylazetidin-3-yl)-7H-pyrrolo[2,3-d]pyrimidin-4-amine

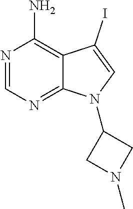 [SMILES: CN1CC(n2cc(I)c3c(N)ncnc32)C1]

Paraformaldehyde (0.149 g, 4.95 mmol) and acetic acid (0.005 g, 0.083 mmol) were added to a solution of 7-(azetidin-3-yl)-5-iodo-7H-pyrrolo[2,3-d]pyrimidin-4-amine (0.520 g, 1.650 mmol) in MeOH (5 mL) and the resulting mixture was stirred at 50° C. for 2 h. NaBH₃CN (0.518 g, 8.25 mmol) was then added and the resulting mixture was stirred at 50° C. for another 12 h. Following completion of the reaction (as indicated by LCMS), the reaction mixture was concentrated under reduced pressure to yield the title compound (0.300 g, crude) which was used without further purification. LCMS: 330.0 [M+H]

### Intermediate A18

### TERT-BUTYL 3-(4-AMINO-5-IODO-7H-PYRROLO[2,3-D]PYRIMIDIN-7-YL)PROPANOATE

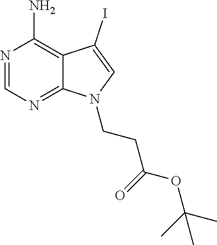 [SMILES: CC(C)(C)OC(=O)CCn1cc(I)c2c(N)ncnc21]

Cs₂CO₃ (2.506 g, 7.69 mmol) and tert-butyl 3-bromopropanoate (0.804 g, 3.85 mmol) were added to a solution of 5-iodo-7H-pyrrolo[2,3-d]pyrimidin-4-amine (1.000 g, 3.85 mmol) in DMF (5 mL) and the resulting mixture was stirred at 90° C. for 12 h. Following completion of the reaction (as indicated by LCMS), the reaction mixture was poured into crushed ice (25 g) and extracted with EtOAc (3×25 mL). The combined organic layers were dried over Na₂SO₄, filtered, and concentrated under reduced pressure to give crude material which was purified by flash chromatography (silica gel 230-400 mesh, eluting with 40% EtOAc in petroleum ether), affording the title compound as a pale brown solid. LCMS: 389.0 [M+H].

### Intermediate B1

### TERT-BUTYL 4-FLUORO-5-(4,4,5,5-TETRAMETHYL-1,3,2-DIOXABOROLAN-2-YL)INDOLINE-1-CARBOXYLATE

### Step 1: Synthesis of 4-fluoroindoline

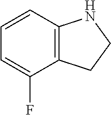

Borane-pyridine complex (2.17 mL, 21.48 mmol) was added to a solution of 4-fluoro-1H-indole (1.00 g, 7.40 mmol) in EtOH (10 mL) at 0° C. and the resulting mixture was stirred for 5 minutes. HCl (6N, 16 mL) was then added dropwise while maintaining the temperature below 10° C. and the reaction mixture was stirred at 0° C. for 3 h. Following completion of the reaction (as indicated by TLC), the pH of the reaction mixture was brought to 8.5 by addition of 10% NaOH before extraction with EtOAc (3×25 mL). The combined organic phases were dried over Na₂SO₄, filtered, and concentrated under reduced pressure to yield the title compound (1.0 g, 97% yield) as pale pink liquid which was taken forward without further purification. ¹H NMR (400 MHz, DMSO-d₆) δ=6.89-6.94 (m, 1H), 6.26-6.32 (m, 2H), 5.79 (bs, 1H), 3.45-3.50 (m, 2H), 2.92-2.96 (m, 3H); LCMS: 138.1 [M+H].

### Step 2: Synthesis of tert-butyl 4-fluoroindoline-1-carboxylate

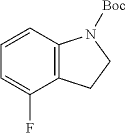 [SMILES: CC(C)(C)OC(=O)N1CCc2c(F)cccc21]

DMAP (0.089 g, 0.72 mmol), DIPEA (0.99 mL, 14.5 mmol), and Boc₂O (1.590 g, 7.29 mmol) were added to a solution of 4-fluoroindoline (1.000 g, 7.29 mmol) in dry DCM (10 mL) at 0° C. The reaction mixture was allowed to warm to 25° C. and was stirred for 16 h. Following completion of the reaction (as indicated by TLC), the mixture was concentrated under reduced pressure to yield crude material which was purified by flash chromatography (silica gel 60-120 mesh, eluting with 4% EtOAc in petroleum ether), giving the title compound as a colorless gum (1.16 g, 68% yield). ¹H NMR (400 MHz, DMSO-d₆) δ=7.47 (bs, 1H), 7.16-7.21 (m, 1H), 6.73-6.78 (m, 1H), 3.93-3.98 (m, 2H), 3.04-3.09 (m, 2H), 1.51 (s, 9H); LCMS: 138.2 [M-Boc].

### Step 3: Synthesis of tert-butyl 5-bromo-4-fluoroindoline-1-carboxylate

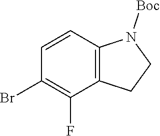 [SMILES: CC(C)(C)OC(=O)N1CCc2c1ccc(Br)c2F]

NBS (1.353 g, 7.6 mmol) was added to a solution of tert-butyl 4-fluoroindoline-1-carboxylate (1.085 g, 7.6 mmol) in dry DCM (10 mL) and the resulting mixture was stirred at 25° C. for 16 h. Following completion of the reaction (as indicated by TLC), the reaction mixture was diluted with DCM (50 mL), washed with brine (2×5 mL), dried over Na₂SO₄, filtered, and concentrated under reduced pressure to give crude material which was purified by flash chromatography (silica gel 230-400 mesh, eluting with 5% EtOAc in petroleum ether), giving the title compound as a yellow solid (1.27 g, 62% yield). ¹H NMR (400 MHz, DMSO-d₆) δ=7.44-7.48 (m, 2H), 3.97-4.01 (m, 2H), 3.11-3.15 (m, 2H), 1.50 (s, 9H); LCMS: 218.0 [M-Boc].

### Step 4: Synthesis of tert-butyl 4-fluoro-5-(4,4,5,5-tetramethyl-1,3,2-dioxaborolan-2-yl)indoline-1-carboxylate

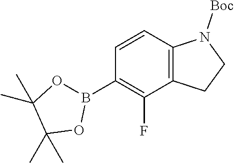 [SMILES: CC(C)(C)OC(=O)N1CCc2c1ccc(B1OC(C)(C)C(C)(C)O1)c2F]

A mixture of B₂pin₂ (0.811 g, 3.10 mmol), AcOK (0.760 g, 7.70 mmol), and tert-butyl 5-bromo-4-fluoroindoline-1-carboxylate (1.010 g, 3.10 mmol) in dioxane (10 mL) was purged with N₂ for 15 minutes. Pd(dppf)Cl₂·DCM (0.126 g, 0.15 mmol) was then added and the resulting mixture was stirred at 100° C. for 16 h. Following completion of the reaction (as indicated by TLC), the solids were removed by filtration through a pad of Celite® (i.e., diatomaceous earth), which was then rinsed with EtOAc (2×5 mL). The combined filtrates were concentrated under reduced pressure to give crude material which was purified by flash chromatography (silica gel 230-400 mesh, eluting with 6% EtOAc in petroleum ether), giving the title compound as a yellow gum (0.72 g, 63% yield). ¹H NMR (400 MHz, DMSO-d₆) δ=7.45-7.49 (m, 2H), 3.95-4.00 (m, 2H), 3.05-3.07 (m, 2H), 1.51 (s, 9H), 1.28 (s, 12H); LCMS: 264.1 [M-Boc].

### Intermediate B2

### TERT-BUTYL 4-CHLORO-5-(4,4,5,5-TETRAMETHYL-1,3,2-DIOXABOROLAN-2-YL)INDOLINE-1-CARBOXYLATE

### Step 1: Synthesis of 4-chloroindoline

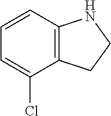

The title compound was prepared via a similar procedure described for step 1 of intermediate B1, starting from 4-chloro-1H-indole (1.00 g, 6.60 mmol), and was obtained as a pale pink liquid (1.00 g, 89% yield) which was taken forward without further purification. ¹H NMR (400 MHz, DMSO-d₆) δ=6.89-6.93 (m, 1H), 6.51 (d, J 7.60 Hz, 1H), 6.41 (d, J 7.60 Hz, 1H), 5.84 (s, 1H), 3.45-3.49 (m, 2H), 2.92-2.97 (m, 2H); LCMS: 154.0 [M+H].

### Step 2: Synthesis of tert-butyl 4-chloroindoline-1-carboxylate

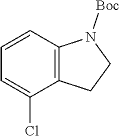 [SMILES: CC(C)(C)OC(=O)N1CCc2c(Cl)cccc21]

The title compound was prepared via a similar procedure described for step 2 of intermediate B1, starting from 4-chloroindoline (0.19 g, 1.24 mmol), and was obtained as a colourless gum (0.15 g, 52% yield). ¹H NMR (400 MHz, DMSO-d₆) δ=7.61 (bs, 1H), 7.17-7.21 (m, 1H), 6.98-7.00 (m, 1H), 3.93-3.98 (m, 2H), 3.05-3.09 (m, 2H), 1.51 (s, 9H); LCMS: 154.2 [M-Boc].

### Step 3: Synthesis of tert-butyl 5-bromo-4-chloroindoline-1-carboxylate

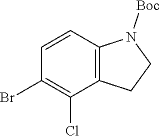 [SMILES: CC(C)(C)OC(=O)N1CCc2c1ccc(Br)c2Cl]

The title compound was prepared via a similar procedure described for step 3 of intermediate B1, starting from tert-butyl 4-chloroindoline-1-carboxylate (0.650 g, 2.56 mmol), and was obtained as a yellow solid (0.70 g, 82% yield). ¹H NMR (400 MHz, DMSO-d₆) δ=7.53-7.55 (m, 2H), 3.95-4.00 (m, 2H), 3.10-3.14 (m, 2H), 1.50 (s, 9H); LCMS: 231.8 [M-Boc].

### Step 4: Synthesis of tert-butyl 4-chloro-5-(4,4,5,5-tetramethyl-1,3,2-dioxaborolan-2-yl)indoline-1-carboxylate

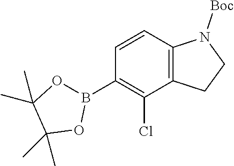 [SMILES: CC(C)(C)OC(=O)N1CCc2c1ccc(B1OC(C)(C)C(C)(C)O1)c2Cl]

The title compound was prepared via a similar procedure described for step 4 of intermediate B1, starting from tert-butyl 5-bromo-4-chloroindoline-1-carboxylate (0.700 g, 2.10 mmol), and was obtained as a yellow gum (0.384 g, 48% yield). ¹H NMR (400 MHz, DMSO-d₆) δ=7.17-7.21 (m, 1H), 6.97-6.99 (m, 1H), 3.93-3.98 (m, 2H), 3.05-3.09 (m, 2H), 1.51 (s, 9H), 1.30 (s, 12H); LCMS: 281.3 [M-Boc].

### General Suzuki-Miyaura Coupling Procedure for the Synthesis of Intermediates C:

A mixture of aryl halide intermediate A1-18 (1.0 eq), boronate ester intermediate B1-2 (1.2 eq), and K₂CO₃ (3.0 eq) in 1,4-dioxane and water (4:1, 20 Vol) was purged with N₂ for 10 min. Pd(PPh₃)₄ (0.05 eq) was then added and the resulting mixture was stirred at 100° C. for 16 h. Following completion of the reaction (as indicated by TLC), the solids were removed by filtration through a pad of Celite® (i.e., diatomaceous earth), which was then rinsed with EtOAc (2×10 mL). The combined filtrates were concentrated under reduced pressure to give crude material which was purified by flash chromatography (silica gel 230-400 mesh, eluting with EtOAc in petroleum ether), giving the desired carbamates C₁-19.

The following intermediates were prepared using the above general procedure.

### Intermediate C1

### TERT-BUTYL 5-(4-AMINO−1-ISOPROPYL-1H-PYRAZOLO[3,4-D]PYRIMIDIN-3-YL)-4-FLUOROINDOLINE-1-CARBOXYLATE

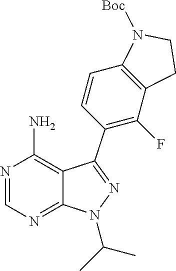 [SMILES: CC(C)n1nc(-c2ccc3c(c2F)CCN3C(=O)OC(C)(C)C)c2c(N)ncnc21]

The title compound was obtained following the general procedure for intermediates C, starting from 3-iodo-1-isopropyl-1H-pyrazolo[3,4-d]pyrimidin-4-amine (A2, 0.500 g, 1.96 mmol) and tert-butyl 4-fluoro-5-(4,4,5,5-tetramethyl-1,3,2-dioxaborolan-2-yl)indoline-1-carboxylate (B1, 0.715 g, 2.36 mmol), and was obtained as a yellow solid (0.230 g, 34% yield). ¹H NMR (400 MHz, DMSO-d₆) δ=8.21 (s, 1H), 7.56-7.65 (m, 1H), 7.31-7.56 (m, 1H), 5.04-5.07 (m, 1H), 4.02-4.07 (m, 2H), 3.14-3.19 (m, 2H), 1.54 (s, 9H), 1.50 (d, J 8.4 Hz, 6H); LCMS: 413.3 [M+H].

### Intermediate C2

### TERT-BUTYL 5-(4-AMINO-1-ISOPROPYL-1H-PYRAZOLO[3,4-D]PYRIMIDIN-3-YL)-4-CHLOROINDOLINE-1-CARBOXYLATE

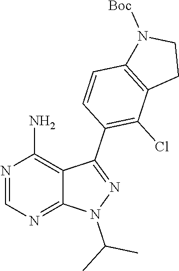 [SMILES: CC(C)n1nc(-c2ccc3c(c2Cl)CCN3C(=O)OC(C)(C)C)c2c(N)ncnc21]

The title compound was obtained following the general procedure for intermediates C, starting from 3-iodo-1-isopropyl-1H-pyrazolo[3,4-d]pyrimidin-4-amine (A2, 0.12 g, 0.39 mmol) and tert-butyl 4-chloro-5-(4,4,5,5-tetramethyl-1,3,2-dioxaborolan-2-yl)indoline-1-carboxylate (B2, 0.18 g, 0.47 mmol), and was obtained as a yellow solid (0.13 g, 77% yield). LCMS: 429.0 [M+H].

### Intermediate C3

### TERT-BUTYL 5-(4-AMINO-1-(OXETAN-3-YL)-1H-PYRAZOLO[3,4-D]PYRIMIDIN-3-YL)-4-FLUOROINDOLINE-1-CARBOXYLATE

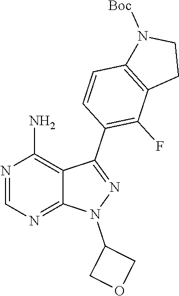 [SMILES: CC(C)(C)OC(=O)N1CCc2c1ccc(-c1nn(C3COC3)c3ncnc(N)c13)c2F]

The title compound was obtained following the general procedure for intermediates C, starting from 3-iodo-1-(oxetan-3-yl)-1H-pyrazolo[3,4-d]pyrimidin-4-amine (A3, 0.176 g, 0.55 mmol) and tert-butyl 4-fluoro-5-(4,4,5,5-tetramethyl-1,3,2-dioxaborolan-2-yl)indoline-1-carboxylate (B1, 0.242 g, 0.66 mmol), and was obtained as a brown solid (0.146 g, 62% yield). ¹H NMR (400 MHz, DMSO-d₆) δ=8.23 (s, 1H), 7.57-7.59 (m, 1H), 7.38-7.42 (m, 1H), 5.99-6.03 (m, 1H), 5.05-5.09 (m, 2H), 4.93-5.01 (m, 2H), 4.04-4.11 (m, 2H), 3.15-3.27 (m, 2H), 1.54 (s, 9H); LCMS: 427.1 [M+H].

### Intermediate C4

### TERT-BUTYL 5-(4-AMINO-1-(OXETAN-3-YL)-1H-PYRAZOLO[3,4-D]PYRIMIDIN-3-YL)-4-CHLOROINDOLINE-1-CARBOXYLATE

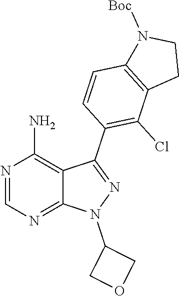 [SMILES: CC(C)(C)OC(=O)N1CCc2c1ccc(-c1nn(C3COC3)c3ncnc(N)c13)c2Cl]

The title compound was obtained following the general procedure for intermediates C, starting from 3-iodo-1-(oxetan-3-yl)-1H-pyrazolo[3,4-d]pyrimidin-4-amine (A3, 0.14 g, 0.44 mmol) and tert-butyl 4-chloro-5-(4,4,5,5-tetramethyl-1,3,2-dioxaborolan-2-yl)indoline-1-carboxylate (B2, 0.20 g, 0.52 mmol), and was obtained as a yellow solid (0.062 g, 32% yield). ¹H NMR (400 MHz, DMSO-d₆) δ=8.23 (s, 1H), 7.56-7.58 (m, 1H), 7.35-7.38 (m, 1H), 5.90-5.93 (m, 1H), 4.93-5.06 (m, 4H), 4.02-4.07 (m, 2H), 3.15-3.19 (m, 2H), 1.54 (s, 9H); LCMS: 443.2 [M+H].

### Intermediate C5

### TERT-BUTYL 5-(4-AMINO−1-CYCLOPROPYL-1H-PYRAZOLO[3,4-D]PYRIMIDIN-3-YL)-4-FLUOROINDOLINE-1-CARBOXYLATE

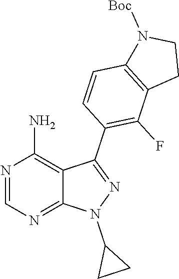 [SMILES: CC(C)(C)OC(=O)N1CCc2c1ccc(-c1nn(C3CC3)c3ncnc(N)c13)c2F]

The title compound was obtained following the general procedure for intermediates C, starting from 1-cyclopropyl-3-iodo-1H-pyrazolo[3,4-d]pyrimidin-4-amine (A1, 0.110 g, 0.34 mmol) and tert-butyl 4-fluoro-5-(4,4,5,5-tetramethyl-1,3,2-dioxaborolan-2-yl)indoline-1-carboxylate (B1, 0.149 g, 0.41 mmol), and was obtained as an off-white solid (0.02 g, 13% yield). ¹H NMR (400 MHz, DMSO-d₆) δ=8.23 (s, 1H), 7.57-7.68 (m, 1H), 7.29-7.33 (m, 1H), 4.02-4.06 (m, 2H), 3.83-3.89 (m, 1H), 3.13-3.18 (m, 2H), 1.53 (s, 9H), 1.17-1.21 (m, 2H), 1.05-1.10 (m, 2H); LCMS: 411.0 [M+H].

### Intermediate C6

### TERT-BUTYL 5-(4-AMINO-1-CYCLOPROPYL-1H-PYRAZOLO[3,4-D]PYRIMIDIN-3-YL)-4-CHLOROINDOLINE-1-CARBOXYLATE

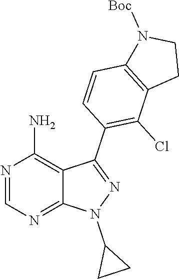 [SMILES: CC(C)(C)OC(=O)N1CCc2c1ccc(-c1nn(C3CC3)c3ncnc(N)c13)c2Cl]

The title compound was obtained following the general procedure for intermediates C, starting from 1-cyclopropyl-3-iodo-1H-pyrazolo[3,4-d]pyrimidin-4-amine (A1, 0.081 g, 0.26 mmol) and tert-butyl 4-chloro-5-(4,4,5,5-tetramethyl-1,3,2-dioxaborolan-2-yl)indoline-1-carboxylate (B2, 0.122 g, 0.32 mmol), and was obtained as a brown solid (0.083 mg, 73% yield). ¹H NMR (400 MHz, DMSO-d₆) δ=8.23 (s, 1H), 7.56-7.58 (m, 1H), 7.27-7.29 (m, 1H), 4.01-4.05 (m, 2H), 3.85-3.88 (m, 1H), 3.12-3.18 (m, 2H), 1.53 (s, 9H), 1.16-1.19 (m, 2H), 1.07-1.10 (m, 2H); LCMS: 427.2 [M+H].

### Intermediate C7

### TERT-BUTYL 5-(4-AMINO-1-(TETRAHYDRO-2H-PYRAN-4-YL)-1H-PYRAZOLO[3,4-D]PYRIMIDIN-3-YL)-4-FLUOROINDOLINE-1-CARBOXYLATE

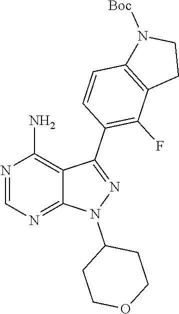 [SMILES: CC(C)(C)OC(=O)N1CCc2c1ccc(-c1nn(C3CCOCC3)c3ncnc(N)c13)c2F]

The title compound was obtained following the general procedure for intermediates C, starting from 3-iodo-1-(tetrahydro-2H-pyran-4-yl)-1H-pyrazolo[3,4-d]pyrimidin-4-amine (A4, 0.232 g, 0.67 mmol) and tert-butyl 4-fluoro-5-(4,4,5,5-tetramethyl-1,3,2-dioxaborolan-2-yl)indoline-1-carboxylate (B1, 0.293 g, 0.80 mmol), and was obtained as a yellow solid (0.13 g, 43% yield). LCMS: 455.2 [M+H].

### Intermediate C8

### TERT-BUTYL 5-(4-AMINO-7-ISOPROPYL-7H-PYRROLO[2,3-D]PYRIMIDIN-5-YL)-4-FLUOROINDOLINE-1-CARBOXYLATE

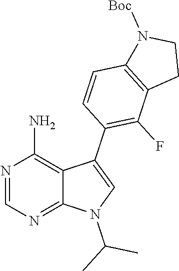 [SMILES: CC(C)n1cc(-c2ccc3c(c2F)CCN3C(=O)OC(C)(C)C)c2c(N)ncnc21]

The title compound was obtained following the general procedure for intermediates C, starting from 5-bromo-7-isopropyl-7H-pyrrolo[2,3-d]pyrimidin-4-amine (A6, 0.234 g, 0.91 mmol) and tert-butyl 4-fluoro-5-(4,4,5,5-tetramethyl-1,3,2-dioxaborolan-2-yl)indoline-1-carboxylate (B1, 0.399 g, 1.09 mmol), and was obtained as a brown solid (0.154 g, 41% yield). LCMS: 412.3 [M+H].

### Intermediate C9

### TERT-BUTYL 5-(4-AMINO-7-CYCLOPROPYL-7H-PYRROLO[2,3-D]PYRIMIDIN-5-YL)-4-FLUOROINDOLINE-1-CARBOXYLATE

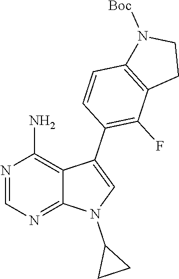 [SMILES: CC(C)(C)OC(=O)N1CCc2c1ccc(-c1cn(C3CC3)c3ncnc(N)c13)c2F]

The title compound was obtained following the general procedure for intermediates C, starting from 7-cyclopropyl-5-iodo-7H-pyrrolo[2,3-d]pyrimidin-4-amine (A5, 0.150 g, 0.500 mmol) and tert-butyl 4-fluoro-5-(4,4,5,5-tetramethyl-1,3,2-dioxaborolan-2-yl)indoline-1-carboxylate (B1, 0.272 g, 0.750 mmol), and was obtained as an off-white solid (0.04 g, 20% yield). LCMS: 410.2 [M+H].

### Intermediate C10

### TERT-BUTYL 5-(4-AMINO-7-CYCLOPROPYL-7H-PYRROLO[2,3-D]PYRIMIDIN-5-YL)-4-CHLOROINDOLINE-1-CARBOXYLATE

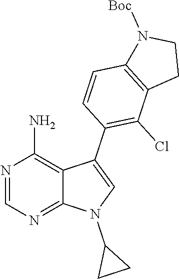 [SMILES: CC(C)(C)OC(=O)N1CCc2c1ccc(-c1cn(C3CC3)c3ncnc(N)c13)c2Cl]

The title compound was obtained following the general procedure for intermediates C, starting from 7-cyclopropyl-5-iodo-7H-pyrrolo[2,3-d]pyrimidin-4-amine (A5, 0.090 g, 0.300 mmol) and tert-butyl 4-chloro-5-(4,4,5,5-tetramethyl-1,3,2-dioxaborolan-2-yl)indoline-1-carboxylate (B2, 0.137 g, 0.360 mmol), and was obtained as a brown gum (0.045 g, 35% yield). LCMS: 426.1 [M+H].

### Intermediate C11

### TERT-BUTYL 5-(4-AMINO-7-(2-HYDROXYETHYL)-7H-PYRROLO[2,3-D]PYRIMIDIN-5-YL)-4-FLUOROINDOLINE-1-CARBOXYLATE

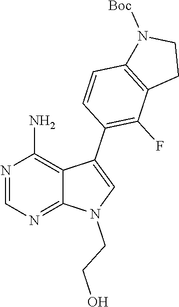 [SMILES: CC(C)(C)OC(=O)N1CCc2c1ccc(-c1cn(CCO)c3ncnc(N)c13)c2F]

The title compound was obtained following the general procedure for intermediates C, starting from 2-(4-amino-5-iodo-7H-pyrrolo[2,3-d]pyrimidin-7-yl)ethan-1-ol (A7, 0.250 g, 0.822 mmol) and tert-butyl 4-fluoro-5-(4,4,5,5-tetramethyl-1,3,2-dioxaborolan-2-yl)indoline-1-carboxylate (B1, 0.358 g, 0.987 mmol), and was obtained as a brown solid (0.030 g, 8.71% yield). LCMS: 414.1 [M+H].

### Intermediate C12

### TERT-BUTYL 5-(4-AMINO-1-(2-HYDROXYETHYL)-1H-PYRAZOLO[3,4-D]PYRIMIDIN-3-YL)-4-FLUOROINDOLINE-1-CARBOXYLATE

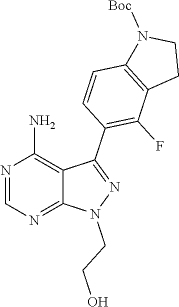 [SMILES: CC(C)(C)OC(=O)N1CCc2c1ccc(-c1nn(CCO)c3ncnc(N)c13)c2F]

The title compound was obtained following the general procedure for intermediates C, starting from 2-(4-amino-3-iodo-1H-pyrazolo[3,4-d]pyrimidin-1-yl)ethan-1-ol (A8, 0.320 g, 1.049 mmol) and tert-butyl 4-fluoro-5-(4,4,5,5-tetramethyl-1,3,2-dioxaborolan-2-yl)indoline-1-carboxylate (B1, 0.381 g, 1.049 mmol), and was obtained as pale yellow gum (0.070 g, 16% yield). LCMS: 415.2 [M+H].

### Intermediate C13

### TERT-BUTYL 5-(4-AMINO-7-TOSYL-7H-PYRROLO[2,3-D]PYRIMIDIN-5-YL)-4-FLUOROINDOLINE-1-CARBOXYLATE

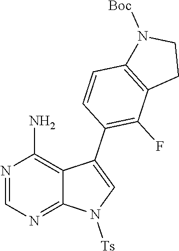 [SMILES: Cc1ccc(S(=O)(=O)n2cc(-c3ccc4c(c3F)CCN4C(=O)OC(C)(C)C)c3c(N)ncnc32)cc1]

The title compound was obtained following the general procedure for intermediates C, starting from 5-iodo-7-tosyl-7H-pyrrolo[2,3-d]pyrimidin-4-amine (A9, 0.100 g, 0.241 mmol) and tert-butyl 4-fluoro-5-(4,4,5,5-tetramethyl-1,3,2-dioxaborolan-2-yl)indoline-1-carboxylate (B1, 0.096 g, 0.266 mmol), and was obtained as pale yellow solid (0.012 g, 8.32% yield). LCMS: 524.1 [M+H].

### Intermediate C14

### TERT-BUTYL 5-(4-AMINO-7-(2-HYDROXY-2-METHYLPROPYL)-7H-PYRROLO[2,3-D]PYRIMIDIN-5-YL)-4-FLUOROINDOLINE-1-CARBOXYLATE

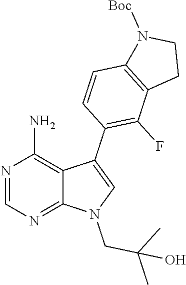 [SMILES: CC(C)(O)Cn1cc(-c2ccc3c(c2F)CCN3C(=O)OC(C)(C)C)c2c(N)ncnc21]

The title compound was obtained following the general procedure for intermediates C, starting from 1-(4-amino-5-iodo-7H-pyrrolo[2,3-d]pyrimidin-7-yl)-2-methylpropan-2-ol (A10, 0.300 g, 0.903 mmol) and tert-butyl 4-fluoro-5-(4,4,5,5-tetramethyl-1,3,2-dioxaborolan-2-yl)indoline-1-carboxylate (B1, 0.328 g, 0.903 mmol), and was obtained as brown gum (0.05 g, 12.5% yield). LCMS: 442.2 [M+H].

### Intermediate C15

### TERT-BUTYL 5-(4-AMINO-1-(2-METHOXYETHYL)-1H-PYRAZOLO[3,4-D]PYRIMIDIN-3-YL)-4-FLUOROINDOLINE-1-CARBOXYLATE

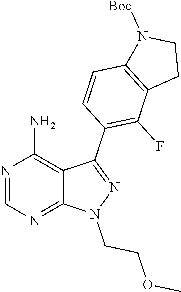 [SMILES: COCCn1nc(-c2ccc3c(c2F)CCN3C(=O)OC(C)(C)C)c2c(N)ncnc21]

The title compound was obtained following the general procedure for intermediates C, starting from 3-iodo-1-(2-methoxyethyl)-1H-pyrazolo[3,4-d]pyrimidin-4-amine (A11, 0.340 g, 1.065 mmol) and tert-butyl 4-fluoro-5-(4,4,5,5-tetramethyl-1,3,2-dioxaborolan-2-yl)indoline-1-carboxylate (B1, 0.299 g, 1.065 mmol), and was obtained as a brown gum (0.040 g, 8.75% yield). LCMS: 429.3 [M+H].

### Intermediate C16

### TERT-BUTYL 5-(4-AMINO-7-(2-METHOXYETHYL)-7H-PYRROLO[2,3-D]PYRIMIDIN-5-YL)-4-FLUOROINDOLINE-1-CARBOXYLATE

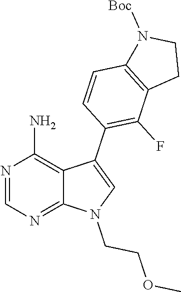 [SMILES: COCCn1cc(-c2ccc3c(c2F)CCN3C(=O)OC(C)(C)C)c2c(N)ncnc21]

The title compound was obtained following the general procedure for intermediates C, starting from 5-iodo-7-(2-methoxyethyl)-7H-pyrrolo[2,3-d]pyrimidin-4-amine (A12, 0.260 g, 0.817 mmol) and tert-butyl 4-fluoro-5-(4,4,5,5-tetramethyl-1,3,2-dioxaborolan-2-yl)indoline-1-carboxylate (B1, 0.297 g, 0.817 mmol), and was obtained as yellow gum (0.146 g, 8.6% yield). LCMS: 428.3 [M+H].

### Intermediate C17

### TERT-BUTYL 5-(4-AMINO-7-(3-(BENZYLOXY)CYCLOBUTYL)-7H-PYRROLO[2,3-D]PYRIMIDIN-5-YL)-4-FLUOROINDOLINE-1-CARBOXYLATE

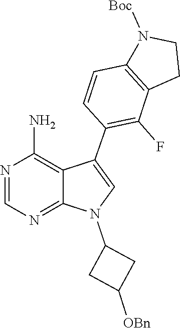 [SMILES: CC(C)(C)OC(=O)N1CCc2c1ccc(-c1cn(C3CC(OCc4ccccc4)C3)c3ncnc(N)c13)c2F]

The title compound was obtained following the general procedure for intermediates C, starting from 7-(3-(benzyloxy)cyclobutyl)-5-iodo-7H-pyrrolo[2,3-d]pyrimidin-4-amine (A13, 0.300 g, 0.428 mmol) and tert-butyl 4-fluoro-5-(4,4,5,5-tetramethyl-1,3,2-dioxaborolan-2-yl)indoline-1-carboxylate (B1, 0.311 g, 0.642 mmol), and was obtained as a brown solid (0.038 g, 16.7% yield). LCMS: 530.3 [M+H].

### Intermediate C18

### TERT-BUTYL 5-(4-AMINO-7-(1-METHYLPIPERIDIN-4-YL)-7H-PYRROLO[2,3-D]PYRIMIDIN-5-YL)-4-FLUOROINDOLINE-1-CARBOXYLATE

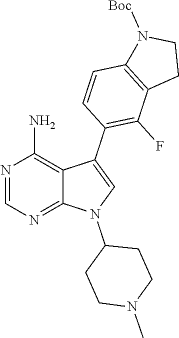 [SMILES: CN1CCC(n2cc(-c3ccc4c(c3F)CCN4C(=O)OC(C)(C)C)c3c(N)ncnc32)CC1]

The title compound was obtained following the general procedure for intermediates C, starting from 5-iodo-7-(1-methylpiperidin-4-yl)-7H-pyrrolo[2,3-d]pyrimidin-4-amine (A14, 0.127 g, 0.300 mmol) and tert-butyl 4-fluoro-5-(4,4,5,5-tetramethyl-1,3,2-dioxaborolan-2-yl)indoline-1-carboxylate (B1, 0.218 g, 0.451 mmol), and was obtained as an off-white gum (0.018 g, 12.9% yield). LCMS: 467.3 [M+H].

### General Procedure for the Synthesis of Intermediates D:

Trifluoroacetic acid (5 Vol) was added to a solution of carbamate intermediate C₁₋₁₈ in dry DCM (10 Vol) at 0° C. and the resulting solution was stirred at 25° C. for 12 h. Following completion of the reaction (as indicated by LCMS), the mixture was concentrated under reduced pressure to give a crude residue which was taken in DCM and washed with aqueous 10% NaHCO₃ and brine. The organic layer was dried over Na₂SO₄, filtered, and concentrated under reduced pressure to yield the desired amine intermediate D1-18 (see Table below) which was used without further purification.

TABLE 1
The following intermediates were prepared using the above general procedure

| | Amine structure | Carbamate structure | |
|---|---|---|---|
| Intermediate | (product) | (starting material) | LC/MS |
| D1 | 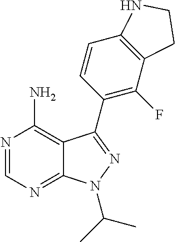 [SMILES: CC(C)n1nc(-c2ccc3c(c2F)CCN3)c2c(N)ncnc21] | 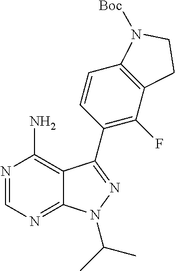 [SMILES: CC(C)n1nc(-c2ccc3c(c2F)CCN3C(=O)OC(C)(C)C)c2c(N)ncnc21] | 313.1 [M + H] |
| D2 | 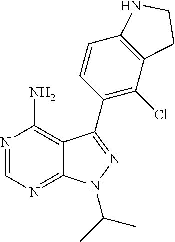 [SMILES: CC(C)n1nc(-c2ccc3c(c2Cl)CCN3)c2c(N)ncnc21] | 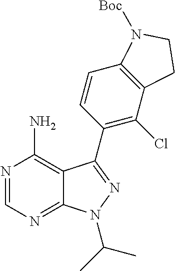 [SMILES: CC(C)n1nc(-c2ccc3c(c2Cl)CCN3C(=O)OC(C)(C)C)c2c(N)ncnc21] | 328.9 [M + H] |
| D3 | 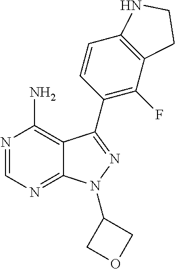 [SMILES: Nc1ncnc2c1c(-c1ccc3c(c1F)CCN3)nn2C1COC1] | 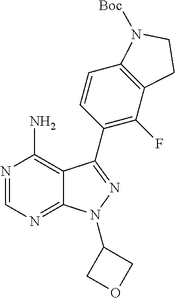 [SMILES: CC(C)(C)OC(=O)N1CCc2c1ccc(-c1nn(C3COC3)c3ncnc(N)c13)c2F] | 327.1 [M + H] |
| D4 | 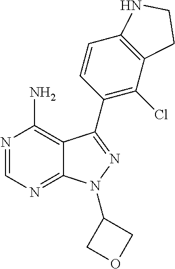 [SMILES: Nc1ncnc2c1c(-c1ccc3c(c1Cl)CCN3)nn2C1COC1] | 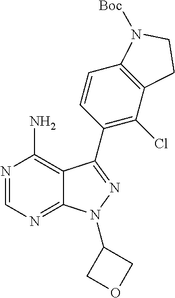 [SMILES: CC(C)(C)OC(=O)N1CCc2c1ccc(-c1nn(C3COC3)c3ncnc(N)c13)c2Cl] | 342.8 [M + H] |
| D5 | 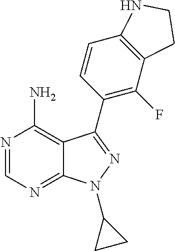 [SMILES: Nc1ncnc2c1c(-c1ccc3c(c1F)CCN3)nn2C1CC1] | 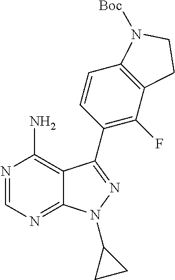 [SMILES: CC(C)(C)OC(=O)N1CCc2c1ccc(-c1nn(C3CC3)c3ncnc(N)c13)c2F] | 311.1 [M + H] |
| D6 | 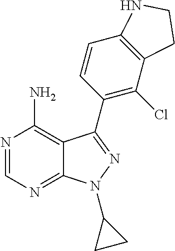 [SMILES: Nc1ncnc2c1c(-c1ccc3c(c1Cl)CCN3)nn2C1CC1] | 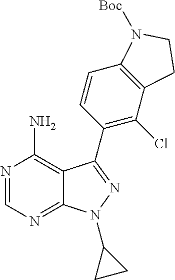 [SMILES: CC(C)(C)OC(=O)N1CCc2c1ccc(-c1nn(C3CC3)c3ncnc(N)c13)c2Cl] | 327.1 [M + H] |
| D7 | 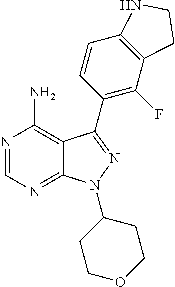 [SMILES: Nc1ncnc2c1c(-c1ccc3c(c1F)CCN3)nn2C1CCOCC1] | 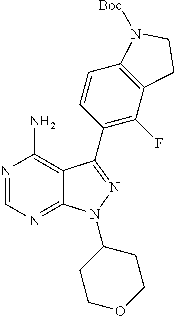 [SMILES: CC(C)(C)OC(=O)N1CCc2c1ccc(-c1nn(C3CCOCC3)c3ncnc(N)c13)c2F] | 355.2 [M + H] |
| D8 | 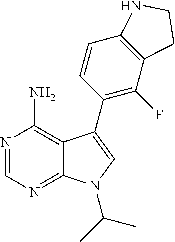 [SMILES: CC(C)n1cc(-c2ccc3c(c2F)CCN3)c2c(N)ncnc21] | 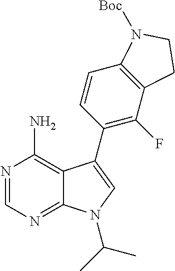 [SMILES: CC(C)n1cc(-c2ccc3c(c2F)CCN3C(=O)OC(C)(C)C)c2c(N)ncnc21] | 312.2 [M + H] |
| D9 | 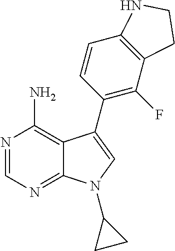 [SMILES: Nc1ncnc2c1c(-c1ccc3c(c1F)CCN3)cn2C1CC1] | 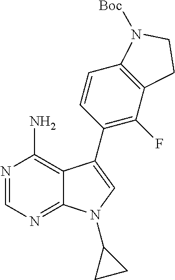 [SMILES: CC(C)(C)OC(=O)N1CCc2c1ccc(-c1cn(C3CC3)c3ncnc(N)c13)c2F] | 310.1 [M + H] |
| D10 | 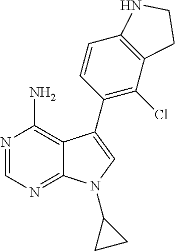 [SMILES: Nc1ncnc2c1c(-c1ccc3c(c1Cl)CCN3)cn2C1CC1] | 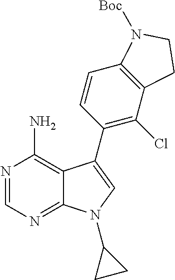 [SMILES: CC(C)(C)OC(=O)N1CCc2c1ccc(-c1cn(C3CC3)c3ncnc(N)c13)c2Cl] | 326.0 [M + H] |
| D11 | 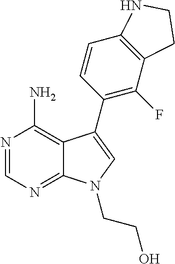 [SMILES: Nc1ncnc2c1c(-c1ccc3c(c1F)CCN3)cn2CCO] | 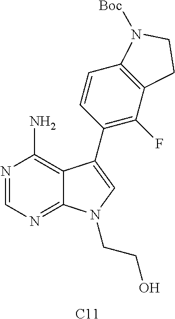 [SMILES: CC(C)(C)OC(=O)N1CCc2c1ccc(-c1cn(CCO)c3ncnc(N)c13)c2F ; "C11"] | 314.1 [M + H] |
| D12 | 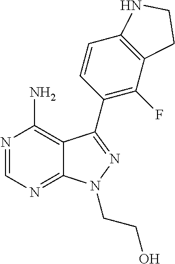 [SMILES: Nc1ncnc2c1c(-c1ccc3c(c1F)CCN3)nn2CCO] | 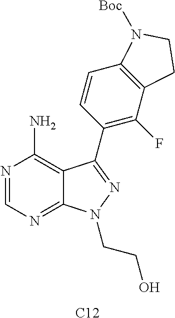 [SMILES: CC(C)(C)OC(=O)N1CCc2c1ccc(-c1nn(CCO)c3ncnc(N)c13)c2F ; "C12"] | 315.2 [M + H] |
| D13 | 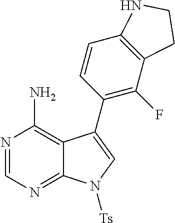 [SMILES: Cc1ccc(S(=O)(=O)n2cc(-c3ccc4c(c3F)CCN4)c3c(N)ncnc32)cc1] | 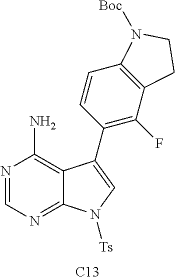 [SMILES: Cc1ccc(S(=O)(=O)n2cc(-c3ccc4c(c3F)CCN4C(=O)OC(C)(C)C)c3c(N)ncnc32)cc1 ; "C13"] | 424.2 [M + H] |
| D14 | 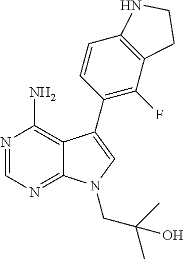 [SMILES: CC(C)(O)Cn1cc(-c2ccc3c(c2F)CCN3)c2c(N)ncnc21] | 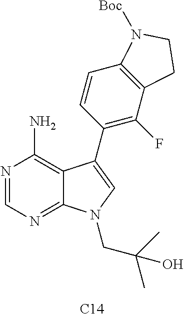 [SMILES: CC(C)(O)Cn1cc(-c2ccc3c(c2F)CCN3C(=O)OC(C)(C)C)c2c(N)ncnc21 ; "C14"] | 342.1 [M + H] |
| D15 | 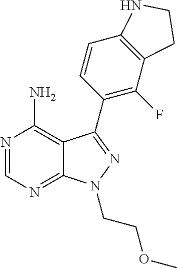 [SMILES: COCCn1nc(-c2ccc3c(c2F)CCN3)c2c(N)ncnc21] | 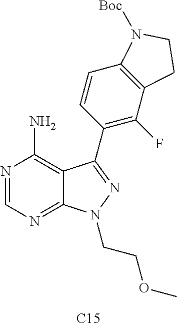 [SMILES: COCCn1nc(-c2ccc3c(c2F)CCN3C(=O)OC(C)(C)C)c2c(N)ncnc21 ; "C15"] | 329.2 [M + H] |
| D16 | 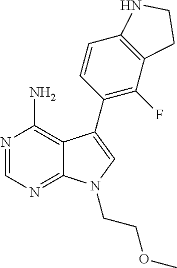 [SMILES: COCCn1cc(-c2ccc3c(c2F)CCN3)c2c(N)ncnc21] | 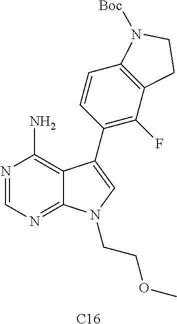 [SMILES: COCCn1cc(-c2ccc3c(c2F)CCN3C(=O)OC(C)(C)C)c2c(N)ncnc21 ; "C16"] | 328.3 [M + H] |
| D17 | 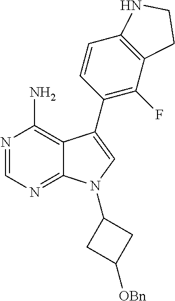 [SMILES: Nc1ncnc2c1c(-c1ccc3c(c1F)CCN3)cn2C1CC(OCc2ccccc2)C1] | 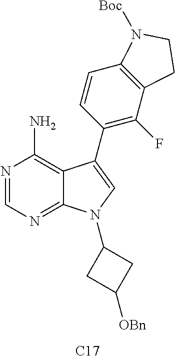 [SMILES: CC(C)(C)OC(=O)N1CCc2c1ccc(-c1cn(C3CC(OCc4ccccc4)C3)c3ncnc(N)c13)c2F ; "C17"] | 430.2 [M + H] |
| D18 | 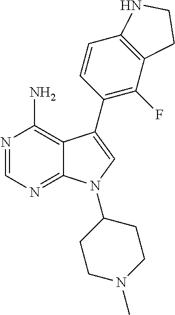 [SMILES: CN1CCC(n2cc(-c3ccc4c(c3F)CCN4)c3c(N)ncnc32)CC1] | 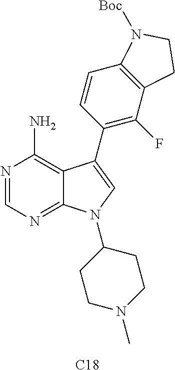 [SMILES: CN1CCC(n2cc(-c3ccc4c(c3F)CCN4C(=O)OC(C)(C)C)c3c(N)ncnc32)CC1 ; "C18"] | 367.3 [M + H] |

### Intermediate E1

### 2-(4-((4-METHYLPIPERAZIN-1-YL)METHYL)-3-(TRIFLUOROMETHYL)PHENYL)ACETIC ACID

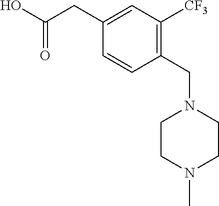 [SMILES: CN1CCN(Cc2ccc(CC(=O)O)cc2C(F)(F)F)CC1]

The title compound was prepared as reported in PCT Publication No. WO 2018/102751 A1.

### Intermediate E2 was Prepared According to the Following Synthetic Scheme:

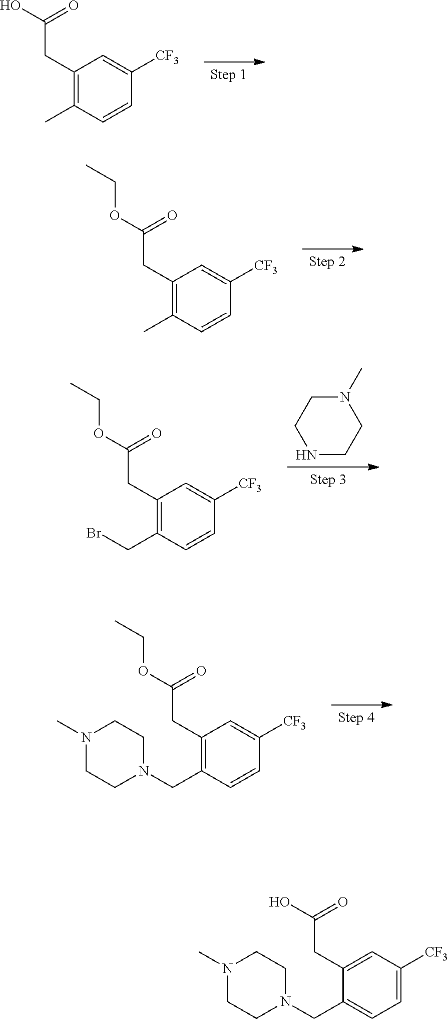

### Intermediate E2

### 2-(2-((4-METHYLPIPERAZIN-1-YL)METHYL)-5-(TRIFLUOROMETHYL)PHENYL)ACETIC ACID

### Step 1: Synthesis of ethyl 2-(2-methyl-5-(trifluoromethyl)phenyl)acetate

H₂SO₄ (0.065 mL, 1.146 mmol) was added to a solution of 2-(2-methyl-5-(trifluoromethyl)phenyl)acetic acid (0.25 g, 1.146 mmol) in ethanol (2.5 mL) at 0° C. and the resulting mixture was stirred at 85° C. for 16 h. Following completion of the reaction (as indicated by TLC), the reaction mixture was concentrated under reduced pressure, giving a residue which was taken in EtOAc (10 mL), washed with aqueous NaHCO₃ (2×5 mL) and brine (5 mL), dried over Na₂SO₄, filtered, and concentrated under reduced pressure to afford the title compound as a pale yellow liquid (0.21 g, 74% yield) which was used without further purification. ¹H NMR (400 MHz, CDCl₃) δ=7.45-7.47 (m, 2H), 7.28-7.32 (m, 1H), 4.17-4.22 (m, 2H), 3.70 (s, 2H), 2.39 (s, 3H), 1.28 (t, J 7.2 Hz, 3H).

Step 2: Synthesis of ethyl 2-(2-(bromomethyl)-5-(trifluoromethyl)phenyl)acetate

AIBN (0.014 g, 0.085 mmol) and NBS (0.152 g, 0.853 mmol) were added to a solution of ethyl 2-(2-methyl-5-(trifluoromethyl)phenyl)acetate (0.210 g, 0.853 mmol) in DCE (10 mL) and the resulting mixture was stirred at 90° C. for 16 h. Following completion of the reaction (as indicated by TLC), the reaction mixture was concentrated under reduced pressure to afford crude material which was purified by flash chromatography (silica gel 230-400 mesh, eluting with 6% EtOAc in petroleum ether), giving the title compound as a pale yellow oil (0.17 g, 61% yield). LCMS: 325.1 [M+H].

### Step 3: Synthesis of ethyl 2-(2-((4-methylpiperazin-1-yl)methyl)-5-(trifluoromethyl) phenyl)acetate

1-Methylpiperazine (0.052 g, 0.523 mmol) and Cs₂CO₃ (0.341 g, 1.046 mmol) were added to a solution of ethyl 2-(2-(bromomethyl)-5-(trifluoromethyl)phenyl) acetate (0.170 g, 0.523 mmol) in THE (7 mL) and the resulting mixture was stirred at 25° C. for 16 h. Following completion of the reaction (as indicated by LCMS), the reaction mixture was filtered through a pad of Celite® (i.e., diatomaceous earth) which was then washed with EtOAc (2×2 mL). The combined filtrates were concentrated under reduced pressure to afford the title compound (0.17 g, crude) which was used without further purification. LCMS: 345.1 [M+H]

### Step 4: Synthesis of 2-(2-((4-methylpiperazin-1-yl)methyl)-5-(trifluoromethyl)phenyl) acetic acid

NaOH (0.020 g, 0.494 mmol) and water (8.89 mg, 0.494 mmol) were added to a stirred of ethyl 2-(2-((4-methylpiperazin-1-yl)methyl)-5-(trifluoromethyl)phenyl) acetate (0.170 g, 0.494 mmol) in THE (5 mL) and the resulting mixture was stirred at 25° C. for 12 h. Following completion of the reaction (as indicated by LCMS), the reaction mixture was concentrated under reduced pressure, giving a residue which was taken in water (2 mL) and washed with EtOAc (2×2 mL). The aqueous layer was acidified to pH=2 with 1.5N HCl and extracted with EtOAc (2×2 mL). The combined organic extracts were dried over Na₂SO₄, filtered, and concentrated under reduced pressure to afford the title compound as an off-white solid (0.14 g, 90% yield). LCMS: 317.1 [M+H].

### Intermediate E3 was Prepared According to the Following Synthetic Scheme:

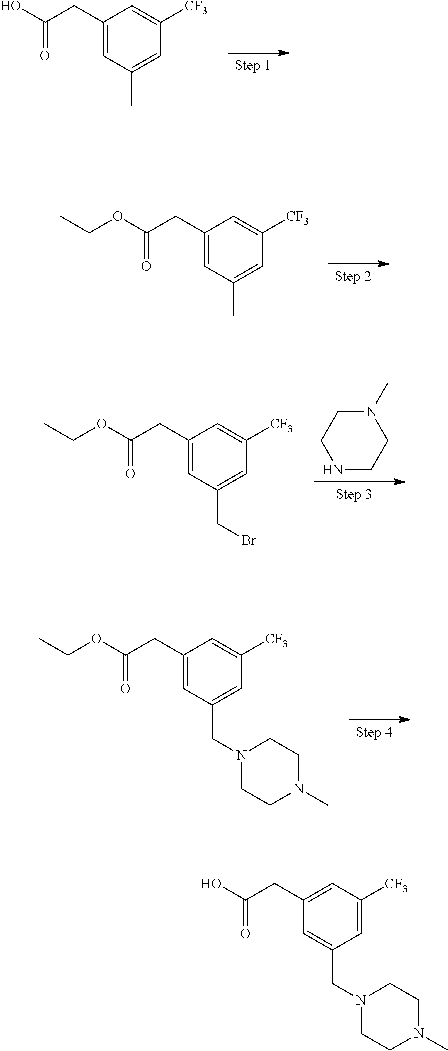

### Intermediate E3

### 2-(3-((4-METHYLPIPERAZIN-1-YL)METHYL)-5-(TRIFLUOROMETHYL)PHENYL)ACETIC ACID

### Step 1: Synthesis of ethyl 2-(3-methyl-5-(trifluoromethyl)phenyl)acetate

The title compound was prepared via a similar procedure described for step 1 of intermediate E2, starting from 2-(2-methyl-5-(trifluoromethyl)phenyl)acetic acid (0.25 g, 1.146 mmol), and was obtained as a pale yellow liquid (0.800 g, 71% yield) which was used without further purification. ¹H NMR (400 MHz, CDCl₃) δ=7.36 (bs, 2H), 7.28-7.31 (m, 1H), 4.17-4.22 (m, 2H), 3.65 (s, 2H), 2.42 (s, 3H), 1.29 (t, J 7.2 Hz, 3H).

### Step 2: Synthesis of ethyl 2-(3-(bromomethyl)-5-(trifluoromethyl)phenyl)acetate

The title compound was prepared via a similar procedure described for step 2 of intermediate E2, starting from ethyl 2-(3-methyl-5-(trifluoromethyl)phenyl)acetate (0.800 g, 3.25 mmol), and was obtained as a pale yellow oil (0.65 g, 61% yield). LCMS: 325.2 [M+H].

### Step 3: Synthesis of ethyl 2-(3-((4-methylpiperazin-1-yl)methyl)-5-(trifluoromethyl) phenyl)acetate

The title compound was prepared via a similar procedure described for step 3 of intermediate E2, starting from 2-(3-(bromomethyl)-5-(trifluoromethyl)phenyl)acetate (0.650 g, 1.999 mmol). The crude material (0.1 g) was used without further purification. LCMS: 345.2 [M+H].

### Step 4: Synthesis of ethyl 2-(3-((4-methylpiperazin-1-yl)methyl)-5-(trifluoromethyl) phenyl)acetate

The title compound was prepared via a similar procedure described for step 4 of intermediate E2, starting from ethyl 2-(3-((4-methylpiperazin-1-yl)methyl)-5-(trifluoromethyl)phenyl)acetate (0.800 g, 2.323 mmol), and was obtained as an off-white solid (0.60 g, 82% yield). LCMS: 317.2 [M+H].

### General Procedure for the Synthesis of Representative Compounds:

Triethylamine (3.0 eq) and TBTU (1.5 eq) were added to a solution of amine intermediate D1-18 (1.0 eq) and carboxylic acid intermediates E (either E1-3 or other commercially available acids, 0.8 eq) in DMF (10 Vol) at 0° C. and the resulting mixture was stirred at 25° C. for 12 h. Following completion of the reaction (as indicated by LCMS), the reaction mixture was concentrated under reduced pressure to give crude material which was purified by reverse-phase preparative HPLC to give the title compounds.

### Example 1

### 1-(5-(4-AMINO−1-ISOPROPYL-1H-PYRAZOLO[3,4-D]PYRIMIDIN-3-YL)-4-FLUOROINDOLIN-1-YL)-2-(3-(TRIFLUOROMETHYL)PHENYL)ETHAN-1-ONE (Compound I-1)

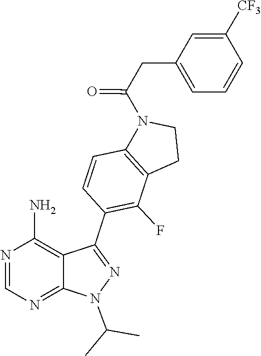 [SMILES: CC(C)n1nc(-c2ccc3c(c2F)CCN3C(=O)Cc2cccc(C(F)(F)F)c2)c2c(N)ncnc21]

The title compound was obtained following the general acid amine coupling procedure, starting from 3-(4-fluoroindolin-5-yl)-1-isopropyl-1H-pyrazolo[3,4-d]pyrimidin-4-amine (D1, 61 mg, 0.19 mmol) and 2-(3-(trifluoromethyl)phenyl)acetic acid (50 mg, 0.24 mmol), and was obtained as an off-white solid (5 mg, 4% yield). ¹H NMR (400 MHz, DMSO-d₆) δ=8.23 (bs, 1H), 7.96 (d, J 8.24 Hz, 1H), 7.53-7.68 (m, 4H), 7.32-7.36 (m, 1H), 5.02-5.09 (m, 1H), 4.36 (t, J 8.52 Hz, 2H), 4.06 (s, 2H), 3.29 (t, J 8.84 Hz, 2H), 1.47 (d, J 6.68 Hz, 6H); LCMS: 499.3 [M+H].

### Example 2

### 1-(5-(4-AMINO−1-ISOPROPYL-1H-PYRAZOLO[3,4-D]PYRIMIDIN-3-YL)-4-FLUOROINDOLIN-1-YL)-2-(4-FLUORO-3-(TRIFLUOROMETHYL)PHENYL)ETHAN-1-ONE (Compound I-2)

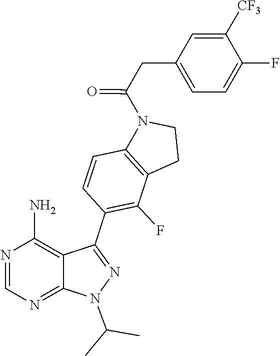 [SMILES: CC(C)n1nc(-c2ccc3c(c2F)CCN3C(=O)Cc2ccc(F)c(C(F)(F)F)c2)c2c(N)ncnc21]

The title compound was obtained following the general acid amine coupling procedure, starting from 3-(4-fluoroindolin-5-yl)-1-isopropyl-1H-pyrazolo[3,4-d]pyrimidin-4-amine (D1, 190 mg, 0.60 mmol) and 2-(4-fluoro-3-(trifluoromethyl)phenyl)acetic acid (108 mg, 0.48 mmol), and was obtained as a white solid (43 mg, 13% yield). ¹H NMR (400 MHz, DMSO-d₆) δ=8.21 (bs, 1H), 7.95 (d, J 8.0 Hz, 1H), 7.72 (d, J 6.8 Hz, 1H), 7.65-7.68 (m, 1H), 7.47-7.52 (m, 1H), 7.32-7.36 (m, 1H), 5.03-5.07 (m, 1H), 4.35 (t, J 8.4 Hz, 2H), 4.05 (s, 2H), 3.29-3.40 (m, 2H), 1.47 (d, J=6.4 Hz, 6H); LCMS: 517.1 [M+H].

### Example 3

### 1-(5-(4-AMINO−1-ISOPROPYL-1H-PYRAZOLO[3,4-D]PYRIMIDIN-3-YL)-4-FLUOROINDOLIN-1-YL)-2-(4-FLUOROPHENYL)ETHAN-1-ONE (Compound I-3)

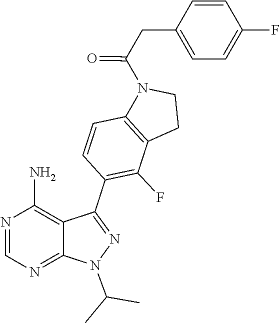 [SMILES: CC(C)n1nc(-c2ccc3c(c2F)CCN3C(=O)Cc2ccc(F)cc2)c2c(N)ncnc21]

The title compound was obtained following the general acid amine coupling procedure, starting from 3-(4-fluoroindolin-5-yl)-1-isopropyl-1H-pyrazolo[3,4-d]pyrimidin-4-amine (D1, 50 mg, 0.16 mmol) and 2-(4-fluorophenyl)acetic acid (25 mg, 0.16 mmol), and was obtained as an off-white solid (7 mg, 10% yield). ¹H NMR (400 MHz, DMSO-d₆) δ=8.36 (bs, 1H), 7.99 (d, J 8.4 Hz, 1H), 7.32-7.40 (m, 3H), 7.12-7.20 (m, 2H), 5.07-5.11 (m, 1H), 4.33 (t, J 8.4 Hz, 2H), 3.92 (s, 2H), 3.26 (t, J 8.4 Hz, 2H), 1.49 (d, J 6.8 Hz, 6H); LCMS: 449.1 [M+H].

### Example 4

### 1-(5-(4-AMINO-1-CYCLOPROPYL-1H-PYRAZOLO[3,4-D]PYRIMIDIN-3-YL)-4-FLUOROINDOLIN-1-YL)-2-(3-(TRIFLUOROMETHYL)PHENYL)ETHAN-1-ONE (Compound I-4)

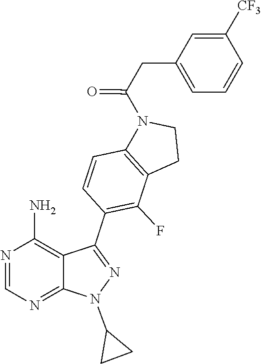 [SMILES: Nc1ncnc2c1c(-c1ccc3c(c1F)CCN3C(=O)Cc1cccc(C(F)(F)F)c1)nn2C1CC1]

The title compound was obtained following the general acid amine coupling procedure, starting from 1-cyclopropyl-3-(4-fluoroindolin-5-yl)-1H-pyrazolo[3,4-d]pyrimidin-4-amine (D5, 43 mg, 0.14 mmol) and 2-(3-(trifluoromethyl)phenyl)acetic acid (30 mg, 0.14 mmol), and was obtained as an off-white solid (4 mg, 6% yield). ¹H NMR (400 MHz, DMSO-d₆) δ=8.24 (bs, 1H), 7.95 (d, J 8.4 Hz, 1H), 7.58-7.69 (m, 4H), 7.31-7.35 (m, 1H), 4.34-4.36 (m, 1H), 4.07 (d, 2H), 3.84-3.88 (m, 2H), 3.26-3.32 (m, 2H), 1.17-1.20 (m, 2H), 1.07-1.10 (m, 2H); LCMS: 497.1 [M+H].

### Example 5

### 1-(5-(4-AMINO−1-ISOPROPYL-1H-PYRAZOLO[3,4-D]PYRIMIDIN-3-YL)-4-FLUOROINDOLIN-1-YL)-2-(4-METHYL-3-(TRIFLUOROMETHYL)PHENYL)ETHAN-1-ONE (Compound I-5)

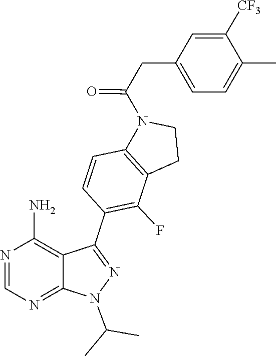 [SMILES: Cc1ccc(CC(=O)N2CCc3c2ccc(-c2nn(C(C)C)c4ncnc(N)c24)c3F)cc1C(F)(F)F]

The title compound was obtained following the general acid amine coupling procedure, starting from 3-(4-fluoroindolin-5-yl)-1-isopropyl-1H-pyrazolo[3,4-d]pyrimidin-4-amine (D1, 50 mg, 0.22 mmol) and 2-(4-methyl-3-(trifluoromethyl)phenyl)acetic acid (68 mg, 0.22 mmol), and was obtained as an off-white solid (3 mg, 4% yield). ¹H NMR (400 MHz, DMSO-d₆) δ=8.21 (bs, 1H), 7.96 (d, J 8.4 Hz, 1H), 7.43-7.47 (m, 3H), 7.32-7.36 (m, 1H), 5.02-5.09 (m, 1H), 4.35 (t, J 8.8 Hz, 2H), 4.01 (s, 2H), 3.29-3.33 (m, 2H), 2.41 (s, 3H), 1.48 (d, J 6.8 Hz, 6H); LCMS: 513.1 [M+H].

### Example 6

### 1-(5-(4-AMINO−1-ISOPROPYL-1H-PYRAZOLO[3,4-D]PYRIMIDIN-3-YL)-4-FLUOROINDOLIN-1-YL)-2-(2-METHYL-3-(TRIFLUOROMETHYL)PHENYL)ETHAN-1-ONE (Compound I-6)

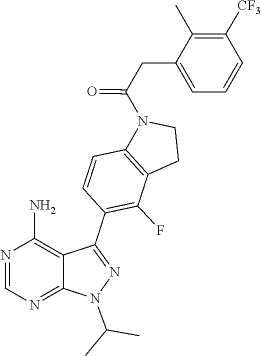 [SMILES: Cc1c(CC(=O)N2CCc3c2ccc(-c2nn(C(C)C)c4ncnc(N)c24)c3F)cccc1C(F)(F)F]

The title compound was obtained following the general acid amine coupling procedure, starting from 3-(4-fluoroindolin-5-yl)-1-isopropyl-1H-pyrazolo[3,4-d]pyrimidin-4-amine (D1, 50 mg, 0.22 mmol) and 2-(2-methyl-3-(trifluoromethyl)phenyl)acetic acid (68 mg, 0.22 mmol), and was obtained as an off-white solid (8.6 mg, 10% yield). ¹H NMR (400 MHz, DMSO-d₆) δ=8.22 (bs, 1H), 7.94 (d, J 8.0 Hz, 1H), 7.62-7.64 (m, 1H), 7.51-7.53 (m, 1H), 7.32-7.40 (m, 2H), 5.04-5.07 (m, 1H), 4.42 (t, J 8.4 Hz, 2H), 4.10 (s, 2H), 3.30-3.32 (m, 2H), 2.33 (s, 3H), 1.48 (d, J=6.8 Hz, 6H); LCMS: 513.2 [M+H].

### Example 7

### 1-(5-(4-AMINO−1-ISOPROPYL-1H-PYRAZOLO[3,4-D]PYRIMIDIN-3-YL)-4-FLUOROINDOLIN-1-YL)-2-(3-(TRIFLUOROMETHOXY)PHENYL)ETHAN-1-ONE (Compound I-7)

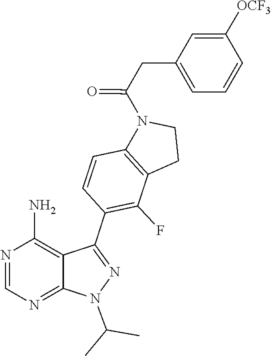 [SMILES: CC(C)n1nc(-c2ccc3c(c2F)CCN3C(=O)Cc2cccc(OC(F)(F)F)c2)c2c(N)ncnc21]

The title compound was obtained following the general acid amine coupling procedure, starting from 3-(4-fluoroindolin-5-yl)-1-isopropyl-1H-pyrazolo[3,4-d]pyrimidin-4-amine (D1, 50 mg, 0.22 mmol) and 2-(3-(trifluoromethoxy)phenyl)acetic acid (68 mg, 0.22 mmol), and was obtained as an off-white solid (3.8 mg, 5% yield). ¹H NMR (400 MHz, DMSO-d₆) δ=8.21 (bs, 1H), 7.97 (d, J 8.4 Hz, 1H), 7.47-7.51 (m, 1H), 7.33-7.36 (m, 3H), 7.27-7.30 (m, 1H), 5.04-5.07 (m, 1H), 4.35 (t, J 8.4 Hz, 2H), 4.02 (s, 2H), 3.26-3.31 (m, 2H), 1.48 (d, J 6.8 Hz, 6H); LCMS: 515.1 [M+H].

### Example 8

### 1-(5-(4-AMINO−1-ISOPROPYL-1H-PYRAZOLO[3,4-D]PYRIMIDIN-3-YL)-4-CHLOROINDOLIN-1-YL)-2-(3-(TRIFLUOROMETHYL)PHENYL)ETHAN-1-ONE (Compound I-8)

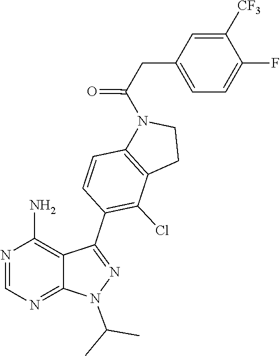 [SMILES: CC(C)n1nc(-c2ccc3c(c2Cl)CCN3C(=O)Cc2ccc(F)c(C(F)(F)F)c2)c2c(N)ncnc21]

The title compound was obtained following the general acid amine coupling procedure, starting from 3-(4-chloroindolin-5-yl)-1-isopropyl-1H-pyrazolo[3,4-d]pyrimidin-4-amine (D2, 0.198 g, 0.30 mmol) and 2-(4-fluoro-3-(trifluoromethyl)phenyl)acetic acid (0.054 g, 0.24 mmol), and was obtained as a white solid (16 mg, 5% yield). ¹H NMR (400 MHz, DMSO-d₆) δ=8.22 (s, 1H), 8.07 (d, J 8.0 Hz, 1H), 7.72-7.75 (m, 1H), 7.66-7.68 (m, 1H), 7.48-7.53 (m, 1H), 7.32 (d, J 8.4 Hz, 1H), 5.03-5.05 (m, 1H), 4.35 (t, J 8.8 Hz, 2H), 4.06 (s, 2H), 3.29 (t, J 8.4 Hz, 2H), 1.48 (d, J=6.8 Hz, 6H); LCMS: 532.8 [M+H].

### Example 9

### 1-(5-(4-AMINO-1-(OXETAN-3-YL)-1H-PYRAZOLO[3,4-D]PYRIMIDIN-3-YL)-4-FLUOROINDOLIN-1-YL)-2-(4-FLUORO-3-(TRIFLUOROMETHYL)PHENYL)ETHAN-1-ONE (Compound I-9)

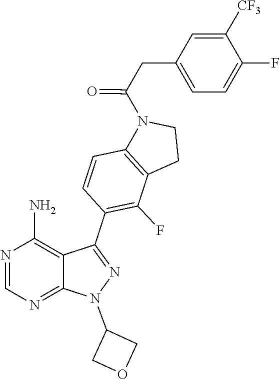 [SMILES: Nc1ncnc2c1c(-c1ccc3c(c1F)CCN3C(=O)Cc1ccc(F)c(C(F)(F)F)c1)nn2C1COC1]

The title compound was obtained following the general acid amine coupling procedure, starting from 3-(4-fluoroindolin-5-yl)-1-(oxetan-3-yl)-1H-pyrazolo[3,4-d]pyrimidin-4-amine (D3, 0.100 g, 0.15 mmol) and 2-(4-fluoro-3-(trifluoromethyl)phenyl)acetic acid (0.026 g, 0.12 mmol), and was obtained as an off-white solid (7 mg, 4% yield). ¹H NMR (400 MHz, DMSO-d₆) δ=8.24 (bs, 1H), 7.98 (d, J 8.4 Hz, 1H), 7.73-7.75 (m, 1H), 7.67-7.70 (m, 1H), 7.48-7.53 (m, 1H), 7.40-7.44 (m, 1H), 5.98-6.05 (m, 1H), 5.07 (t, J 6.4 Hz, 2H), 4.99 (t, J 6.8 Hz, 2H), 4.37 (t, J 8.4 Hz, 2H), 4.06 (s, 2H), 3.26-3.31 (m, 2H); LCMS: 530.8 [M+H].

### Example 10

### 1-(5-(4-AMINO-1-(OXETAN-3-YL)-1H-PYRAZOLO[3,4-D]PYRIMIDIN-3-YL)-4-CHLOROINDOLIN-1-YL)-2-(4-FLUORO-3-(TRIFLUOROMETHYL)PHENYL)ETHAN-1-ONE (Compound I-10)

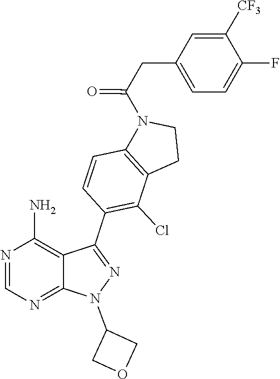 [SMILES: Nc1ncnc2c1c(-c1ccc3c(c1Cl)CCN3C(=O)Cc1ccc(F)c(C(F)(F)F)c1)nn2C1COC1]

The title compound was obtained following the general acid amine coupling procedure, starting from 3-(4-chloroindolin-5-yl)-1-(oxetan-3-yl)-1H-pyrazolo[3,4-d]pyrimidin-4-amine (D4, 0.127 g, 0.18 mmol) and 2-(4-fluoro-3-(trifluoromethyl)phenyl)acetic acid (0.032 g, 0.14 mmol), and was obtained as a white solid (9 mg, 5% yield). ¹H NMR (400 MHz, DMSO-d₆) δ=8.23 (s, 1H), 8.08-8.10 (m, 1H), 7.73-7.74 (m, 1H), 7.66-7.70 (m, 1H), 7.48-7.50 (m, 1H), 7.38 (d, J 8.0 Hz, 1H), 5.99-6.03 (m, 1H), 5.06 (t, J 6.4 Hz, 2H), 5.00 (t, J 6.4 Hz, 2H), 4.36 (t, J 8.4 Hz, 2H), 4.06 (s, 2H), 3.30 (t, J 8.4 Hz, 2H); LCMS: 546.8 [M+H].

### Example 11

### 1-(5-(4-AMINO−1-ISOPROPYL-1H-PYRAZOLO[3,4-D]PYRIMIDIN-3-YL)-4-FLUOROINDOLIN-1-YL)-2-(2-FLUORO-5-(TRIFLUOROMETHYL)PHENYL)ETHAN-1-ONE (Compound I-11)

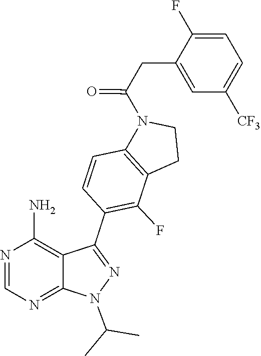 [SMILES: CC(C)n1nc(-c2ccc3c(c2F)CCN3C(=O)Cc2cc(C(F)(F)F)ccc2F)c2c(N)ncnc21]

The title compound was obtained following the general acid amine coupling procedure, starting from 3-(4-fluoroindolin-5-yl)-1-isopropyl-1H-pyrazolo[3,4-d]pyrimidin-4-amine (D1, 0.100 g, 0.32 mmol) and 2-(2-fluoro-5-(trifluoromethyl)phenyl)acetic acid (0.056 g, 0.25 mmol), and was obtained as a white solid (23 mg, 14% yield). ¹H NMR (400 MHz, DMSO-d₆) δ=8.21 (s, 1H), 7.92 (d, J 8.0 Hz, 1H), 7.75-7.83 (m, 2H), 7.44-7.49 (m, 1H), 7.32-7.36 (m, 1H), 5.04-5.07 (m, 1H), 4.41 (t, J 8.4 Hz, 2H), 4.11 (s, 2H), 3.30-3.32 (m, 2H), 1.48 (d, J 6.8 Hz, 6H); LCMS: 517.2 [M+H].

### Example 12

### 1-(5-(4-AMINO-1-(OXETAN-3-YL)-1H-PYRAZOLO[3,4-D]PYRIMIDIN-3-YL)-4-FLUOROINDOLIN-1-YL)-2-(2-FLUORO-5-(TRIFLUOROMETHYL)PHENYL)ETHAN-1-ONE (Compound I-12)

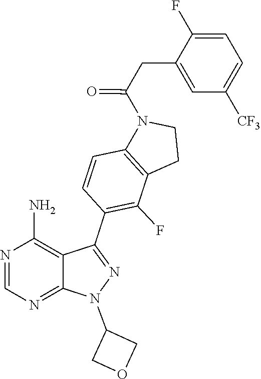 [SMILES: Nc1ncnc2c1c(-c1ccc3c(c1F)CCN3C(=O)Cc1cc(C(F)(F)F)ccc1F)nn2C1COC1]

The title compound was obtained following the general acid amine coupling procedure, starting from 3-(4-fluoroindolin-5-yl)-1-(oxetan-3-yl)-1H-pyrazolo[3,4-d]pyrimidin-4-amine (D3, 0.24 g, 0.73 mmol) and 2-(2-fluoro-5-(trifluoromethyl)phenyl)acetic acid (0.13 g, 0.58 mmol), and was obtained as a white solid (9 mg, 2% yield). ¹H NMR (400 MHz, DMSO-d₆) δ=8.23 (s, 1H), 7.95 (d, J 8.0 Hz, 1H), 7.82 (d, J 6.4 Hz, 1H), 7.76-7.77 (m, 1H), 7.39-7.47 (m, 2H), 5.99-6.03 (m, 1H), 5.06 (t, J 6.4 Hz, 2H), 4.99 (t, J 6.8 Hz, 2H), 4.42 (t, J 8.4 Hz, 2H), 4.11 (s, 2H), 3.33 (t, J 8.4 Hz, 2H); LCMS: 531.1 [M+H].

### Example 13

### 1-(5-(4-AMINO-7-ISOPROPYL-7H-PYRROLO[2,3-D]PYRIMIDIN-5-YL)-4-FLUOROINDOLIN-1-YL)-2-(4-FLUORO-3-(TRIFLUOROMETHYL)PHENYL)ETHAN-1-ONE (Compound I-13)

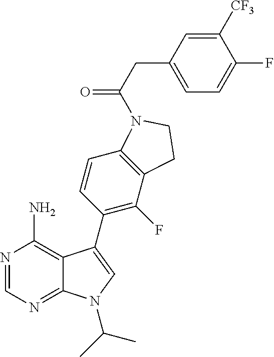 [SMILES: CC(C)n1cc(-c2ccc3c(c2F)CCN3C(=O)Cc2ccc(F)c(C(F)(F)F)c2)c2c(N)ncnc21]

The title compound was obtained following the general acid amine coupling procedure, starting from 5-(4-fluoroindolin-5-yl)-7-isopropyl-7H-pyrrolo[2,3-d]pyrimidin-4-amine (D9, 0.14 g, 0.45 mmol) and 2-(4-fluoro-3-(trifluoromethyl)phenyl)acetic acid (0.10 g, 0.45 mmol), and was obtained as an off-white solid (52 mg, 64% yield). ¹H NMR (400 MHz, DMSO-d₆) δ=8.13 (bs, 1H), 7.91 (d, J 8.4 Hz, 1H), 7.72-7.74 (m, 1H), 7.65-7.69 (m, 1H), 7.48-7.52 (m, 1H), 7.42 (bs, 1H), 7.20-7.24 (m, 1H), 6.04 (s, 2H), 4.94-5.01 (m, 1H), 4.34 (t, J 8.4 Hz, 2H), 4.03 (s, 2H), 3.28 (t, J 8.4 Hz, 2H), 1.46 (d, J 6.8 Hz, 6H); LCMS: 516.2 [M+H].

### Example 14

### 1-(5-(4-AMINO-1-(TETRAHYDRO-2H-PYRAN-4-YL)-1H-PYRAZOLO[3,4-D]PYRIMIDIN-3-YL)-4-FLUOROINDOLIN-1-YL)-2-(4-FLUORO-3-(TRIFLUOROMETHYL)PHENYL)ETHAN-1-ONE (Compound I-14)

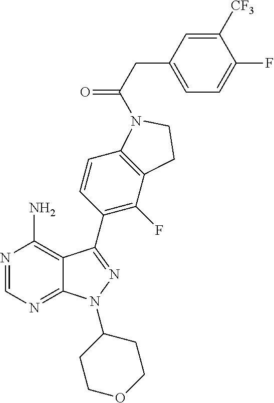 [SMILES: Nc1ncnc2c1c(-c1ccc3c(c1F)CCN3C(=O)Cc1ccc(F)c(C(F)(F)F)c1)nn2C1CCOCC1]

The title compound was obtained following the general acid amine coupling procedure, starting from 3-(4-fluoroindolin-5-yl)-1-(tetrahydro-2H-pyran-4-yl)-1H-pyrazolo[3,4-d]pyrimidin-4-amine (D7, 0.12 g, 0.34 mmol) and 2-(4-fluoro-3-(trifluoromethyl)phenyl)acetic acid (0.06 g, 0.27 mmol), and was obtained as a white solid (23 mg, 12% yield). ¹H NMR (400 MHz, DMSO-d₆) δ=8.23 (bs, 1H), 7.93 (d, J 8.4 Hz, 1H), 7.82-7.83 (m, 1H), 7.75-7.79 (m, 1H), 7.45-7.49 (m, 1H), 7.33-7.37 (m, 1H), 4.90-4.96 (m, 1H), 4.41 (t, J 8.4 Hz, 2H), 4.11 (s, 2H), 3.98-4.02 (m, 2H), 3.54-3.57 (m, 2H), 3.30-3.37 (m, 2H), 2.12-2.23 (m, 2H), 1.87-1.91 (m, 2H); LCMS: 559.1 [M+H].

### Example 15

### 1-(5-(4-AMINO−1-CYCLOPROPYL-1H-PYRAZOLO[3,4-D]PYRIMIDIN-3-YL)-4-CHLOROINDOLIN-1-YL)-2-(2-FLUORO-5-(TRIFLUOROMETHYL)PHENYL)ETHAN-1-ONE (Compound I-15)

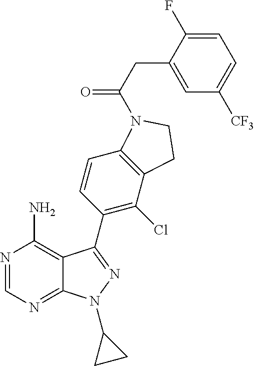 [SMILES: Nc1ncnc2c1c(-c1ccc3c(c1Cl)CCN3C(=O)Cc1cc(C(F)(F)F)ccc1F)nn2C1CC1]

The title compound was obtained following the general acid amine coupling procedure, starting from 3-(4-chloroindolin-5-yl)-1-cyclopropyl-1H-pyrazolo[3,4-d]pyrimidin-4-amine (D6, 63 mg, 0.193 mmol) and 2-(2-fluoro-5-(trifluoromethyl)phenyl)acetic acid (34 mg, 0.150 mmol), and was obtained as a white solid (27 mg, 26% yield). ¹H NMR (400 MHz, DMSO-d₆) δ=8.24 (s, 1H), 8.02 (d, J 8.4 Hz, 1H), 7.77-7.83 (m, 2H), 7.45-7.49 (m, 1H), 7.29 (d, J 8.0 Hz, 1H), 4.40 (t, J 8.4 Hz, 2H), 4.11 (s, 2H), 3.85-3.89 (m, 1H), 3.28-3.30 (m, 2H), 1.16-1.17 (m, 2H), 1.07-1.08 (m, 2H); LCMS: 531.1 [M+H].

### Example 16

### 1-(5-(4-AMINO-7-CYCLOPROPYL-7H-PYRROLO[2,3-D]PYRIMIDIN-5-YL)-4-CHLOROINDOLIN-1-YL)-2-(2-FLUORO-5-(TRIFLUOROMETHYL)PHENYL)ETHAN-1-ONE (Compound I-16)

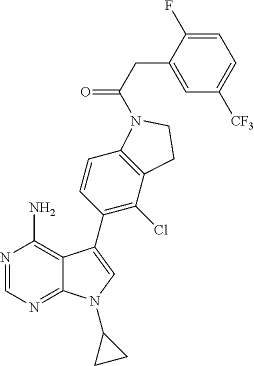 [SMILES: Nc1ncnc2c1c(-c1ccc3c(c1Cl)CCN3C(=O)Cc1cc(C(F)(F)F)ccc1F)cn2C1CC1]

The title compound was obtained following the general acid amine coupling procedure, starting from 5-(4-chloroindolin-5-yl)-7-cyclopropyl-7H-pyrrolo[2,3-d]pyrimidin-4-amine (D10, 0.140 g, 0.430 mmol) and 2-(2-fluoro-5-(trifluoromethyl)phenyl)acetic acid (0.076 g, 0.344 mmol), and was obtained as an off-white solid (14 mg, 6% yield). ¹H NMR (400 MHz, DMSO-d₆) δ=8.14 (bs, 1H), 7.97 (d, J 8.4 Hz, 1H), 7.80-7.82 (m, 1H), 7.75-7.78 (m, 1H), 7.46 (t, J 8.8 Hz, 1H), 7.22 (d, J 8.0 Hz, 1H), 7.16 (bs, 1H), 5.87 (bs, 2H), 4.37 (t, J 8.8 Hz, 2H), 4.08 (s, 2H), 3.55-3.59 (m, 1H), 3.28 (t, J 8.8 Hz, 2H), 1.02-1.03 (m, 4H); LCMS: 530.2 [M+H].

### Example 17

### 1-(5-(4-AMINO-7-CYCLOPROPYL-7H-PYRROLO[2,3-D]PYRIMIDIN-5-YL)-4-FLUOROINDOLIN-1-YL)-2-(2-FLUORO-5-(TRIFLUOROMETHYL)PHENYL)ETHAN-1-ONE (Compound I-17)

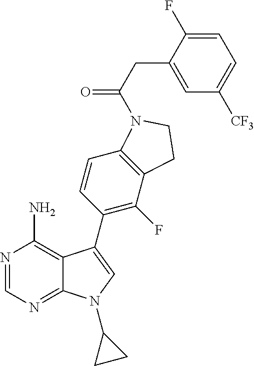 [SMILES: Nc1ncnc2c1c(-c1ccc3c(c1F)CCN3C(=O)Cc1cc(C(F)(F)F)ccc1F)cn2C1CC1]

The title compound was obtained following the general acid amine coupling procedure, starting from 7-cyclopropyl-5-(4-fluoroindolin-5-yl)-7H-pyrrolo[2,3-d]pyrimidin-4-amine (D9, 56 mg, 0.181 mmol) and 2-(2-fluoro-5-(trifluoromethyl)phenyl)acetic acid (40 mg, 0.181 mmol), and was obtained as an off-white solid (9 mg, 10% yield). ¹H NMR (400 MHz, DMSO-d₆) δ=8.15 (bs, 1H), 7.86 (d, J 8.0 Hz, 1H), 7.81-7.82 (m, 1H), 7.75-7.78 (m, 1H), 7.46 (t, J 9.2 Hz, 1H), 7.18-7.22 (m, 2H), 6.01 (bs, 2H), 4.38 (t, J 8.4 Hz, 2H), 4.08 (s, 2H), 3.54-3.60 (m, 1H), 3.30 (t, J 8.4 Hz, 2H), 1.01-1.04 (m, 4H); LCMS: 514.1 [M+H].

### Example 18

### 1-(5-(4-AMINO-7-(2-HYDROXYETHYL)-7H-PYRROLO[2,3-D]PYRIMIDIN-5-YL)-4-FLUOROINDOLIN-1-YL)-2-(2-FLUORO-5-(TRIFLUOROMETHYL)PHENYL)ETHAN-1-ONE (Compound I-18)

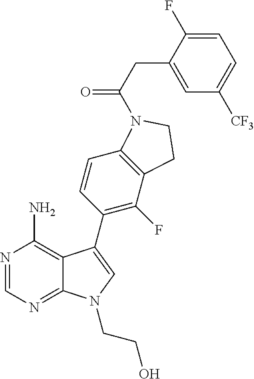 [SMILES: Nc1ncnc2c1c(-c1ccc3c(c1F)CCN3C(=O)Cc1cc(C(F)(F)F)ccc1F)cn2CCO]

The title compound was obtained following the general acid amine coupling procedure, starting from 2-(4-amino-5-(4-fluoroindolin-5-yl)-7H-pyrrolo[2,3-d]pyrimidin-7-yl)ethan-1-ol (D11, 23 mg, 0.073 mmol) and 2-(2-fluoro-(trifluoromethyl)phenyl)acetic acid (16 mg, 0.073 mmol), and was obtained as an off-white solid (20 mg, 52% yield). ¹H NMR (400 MHz, DMSO-d₆) δ=8.14 (s, 1H), 7.88 (d, J 8.4 Hz, 1H), 7.81-7.83 (m, 1H), 7.75-7.78 (m, 1H), 7.47 (t, J 8.8 Hz, 1H), 7.30 (s, 1H), 7.20 (t, J 8.0 Hz, 1H), 6.05 (bs, 2H), 4.97 (t, J 5.2 Hz, 1H), 4.39 (t, J 8.8 Hz, 2H), 4.23 (t, J 5.6 Hz, 2H), 4.09 (s, 2H), 3.73-3.77 (m, 2H), 3.29-3.31 (m, 2H); LCMS: 518.2 [M+H].

### Example 19

### 1-(5-(4-AMINO-1-(2-HYDROXYETHYL)-1H-PYRAZOLO[3,4-D]PYRIMIDIN-3-YL)-4-FLUOROINDOLIN-1-YL)-2-(2-FLUORO-5-(TRIFLUOROMETHYL)PHENYL)ETHAN-1-ONE (Compound I-19)

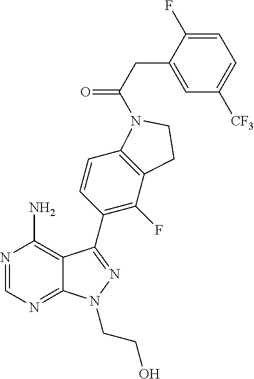 [SMILES: Nc1ncnc2c1c(-c1ccc3c(c1F)CCN3C(=O)Cc1cc(C(F)(F)F)ccc1F)nn2CCO]

The title compound was obtained following the general acid amine coupling procedure, starting from 2-(4-amino-3-(4-fluoroindolin-5-yl)-1H-pyrazolo[3,4-d]pyrimidin-1-yl)ethan-1-ol (D12, 70 mg, 0.223 mmol) and 2-(2-fluoro-(trifluoromethyl)phenyl)acetic acid (49 mg, 0.223 mmol), and was obtained as an off-white solid (3 mg, 2.4% yield). ¹H NMR (400 MHz, DMSO-d₆) δ=8.32 (s, 1H), 7.94 (d, J 8.4 Hz, 1H), 7.77-7.83 (m, 2H), 7.47 (t, J 8.8 Hz, 1H), 7.37 (t, J 8.0 Hz, 1H), 4.39-4.44 (m, 4H), 4.12 (s, 2H), 3.85 (t, J 5.6 Hz, 2H), 3.29-3.33 (m, 2H); LCMS: 519.2 [M+H].

### Example 20

### 1-(5-(4-AMINO-7-CYCLOPROPYL-7H-PYRROLO[2,3-D]PYRIMIDIN-5-YL)-4-FLUOROINDOLIN-1-YL)-2-(6-(TRIFLUOROMETHYL)PYRIDIN-2-YL)ETHAN-1-ONE (Compound I-20)

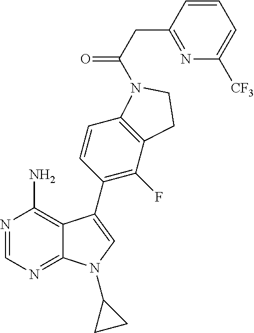 [SMILES: Nc1ncnc2c1c(-c1ccc3c(c1F)CCN3C(=O)Cc1cccc(C(F)(F)F)n1)cn2C1CC1]

The title compound was obtained following the general acid amine coupling procedure, starting from 7-cyclopropyl-5-(4-fluoroindolin-5-yl)-7H-pyrrolo[2,3-d]pyrimidin-4-amine (D9, 22 mg, 0.071 mmol) and 2-(6-(trifluoromethyl)pyridin-2-yl)acetic acid (15 mg, 0.071 mmol), and was obtained as an off-white solid (6 mg, 16% yield). ¹H NMR (400 MHz, DMSO-d₆) δ=8.36 (s, 1H), 8.08-8.12 (m, 1H), 7.90 (d, J 8.0 Hz, 1H), 7.84 (d, J 7.6 Hz, 1H), 7.71 (d, J 8.0 Hz, 1H), 7.47 (s, 1H), 7.23 (t, J 8.0 Hz, 1H), 4.39 (t, J 8.8 Hz, 2H), 4.24 (s, 2H), 3.65-3.71 (m, 1H), 3.26 (t, J 8.8 Hz, 2H), 1.07-1.09 (m, 4H); LCMS: 497.1 [M+H].

### Example 21

### 1-(5-(4-AMINO-7-CYCLOPROPYL-7H-PYRROLO[2,3-D]PYRIMIDIN-5-YL)-4-FLUOROINDOLIN-1-YL)-2-(6-METHYLPYRIDIN-2-YL)ETHAN-1-ONE (Compound I-21)

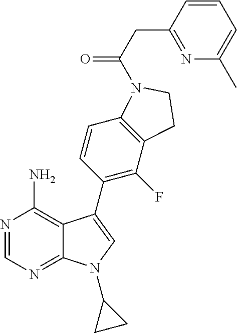 [SMILES: Cc1cccc(CC(=O)N2CCc3c2ccc(-c2cn(C4CC4)c4ncnc(N)c24)c3F)n1]

The title compound was obtained following the general acid amine coupling procedure, starting from 7-cyclopropyl-5-(4-fluoroindolin-5-yl)-7H-pyrrolo[2,3-d]pyrimidin-4-amine (D9, 22 mg, 0.071 mmol) and 2-(6-methylpyridin-2-yl)acetic acid (11 mg, 0.071 mmol), and was obtained as pale brown solid (2 mg, 5.4% yield). ¹H NMR (400 MHz, CD₃OD) δ=8.35 (s, 1H), 8.30 (t, J 8.0 Hz, 1H), 8.01 (d, J 8.4 Hz, 1H), 7.72 (t, J 7.2 Hz, 1H), 7.46 (s, 1H), 7.31 (t, J 8.0 Hz, 1H), 4.45 (t, J 8.4 Hz, 2H), 3.69-3.72 (m, 2H), 3.41-3.43 (m, 2H), 2.78 (s, 3H), 1.18-1.25 (m, 4H); LCMS: 443.6 [M+H].

### Example 21

### 1-(5-(4-AMINO-7-CYCLOPROPYL-7H-PYRROLO[2,3-D]PYRIMIDIN-5-YL)-4-FLUOROINDOLIN-1-YL)-2-(6-METHYLPYRIDIN-2-YL)ETHAN-1-ONE (Compound I-21)

### Step 1: Synthesis of 1-(5-(4-amino-7-tosyl-7H-pyrrolo[2,3-d]pyrimidin-5-yl)-4-fluoroindolin-1-yl)-2-(2-fluoro-5-(trifluoromethyl)phenyl)ethan-1-one

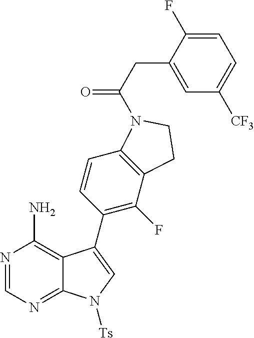 [SMILES: Cc1ccc(S(=O)(=O)n2cc(-c3ccc4c(c3F)CCN4C(=O)Cc3cc(C(F)(F)F)ccc3F)c3c(N)ncnc32)cc1]

The title compound was obtained following the general acid amine coupling procedure, starting from 5-(4-fluoroindolin-5-yl)-7-tosyl-7H-pyrrolo[2,3-d]pyrimidin-4-amine (D13, 25 mg, 0.059 mmol) and 2-(2-fluoro-5-(trifluoromethyl)phenyl)acetic acid (16 mg, 0.071 mmol), and was obtained as brown solid (14 mg, 26% yield). LCMS: 628.2 [M+H].

### Step 2: Synthesis of 1-(5-(4-amino-7H-pyrrolo[2,3-d]pyrimidin-5-yl)-4-fluoroindolin-1-yl)-2-(2-fluoro-5-(trifluoromethyl)phenyl)ethan-1-one

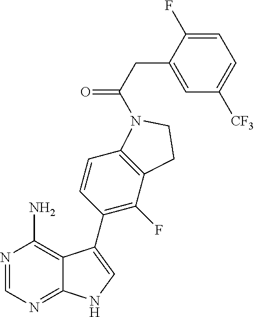 [SMILES: Nc1ncnc2[nH]cc(-c3ccc4c(c3F)CCN4C(=O)Cc3cc(C(F)(F)F)ccc3F)c12]

KOH (5.4 mg, 0.096 mmol) was added to a solution of 1-(5-(4-amino-7-tosyl-7H-pyrrolo[2,3-d]pyrimidin-5-yl)-4-fluoroindolin-1-yl)-2-(2-fluoro-5-(trifluoromethyl)phenyl)ethan-1-one (20 mg, 0.032 mmol) in water (3 mL) at 0° C. and the resulting mixture was stirred at 60° C. for 2 h. Following completion of the reaction (as indicated by LCMS), the reaction mixture was diluted with water (5 mL) and extracted with DCM (2×10 mL). The combined organic extracts were washed with brine, dried over Na₂SO₄, filtered, and concentrated under reduced pressure to give crude material which was purified by preparative HPLC (eluting with 10 mM NH₄OAc in water and acetonitrile), affording the title compound as a white solid (2.4 mg, 15% yield). ¹H NMR (400 MHz, DMSO-d₆) δ=11.84 (s, 1H), 8.10 (s, 1H), 7.87 (d, J 8.0 Hz, 1H), 7.82 (d, J 6.8 Hz, 1H), 7.75-7.78 (m, 1H), 7.47 (t, J 9.2 Hz, 1H), 7.19-7.23 (m, 2H), 5.96 (bs, 2H), 4.39 (t, J 8.8 Hz, 2H), 4.09 (s, 2H), 3.28-3.31 (m, 2H); LCMS: 474.2 [M+H].

### Example 23

### 1-(5-(4-AMINO-7-(2-HYDROXY-2-METHYLPROPYL)-7H-PYRROLO[2,3-D]PYRIMIDIN-5-YL)-4-FLUOROINDOLIN-1-YL)-2-(2-FLUORO-5-(TRIFLUOROMETHYL)PHENYL)ETHAN-1-ONE (Compound I-23)

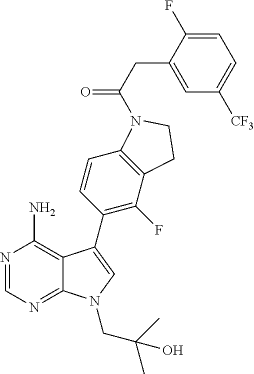 [SMILES: CC(C)(O)Cn1cc(-c2ccc3c(c2F)CCN3C(=O)Cc2cc(C(F)(F)F)ccc2F)c2c(N)ncnc21]

The title compound was obtained following the general acid amine coupling procedure, starting from 1-(4-amino-5-(4-fluoroindolin-5-yl)-7H-pyrrolo[2,3-d]pyrimidin-7-yl)-2-methylpropan-2-ol (D14, 35 mg, 0.103 mmol) and 2-(2-fluoro-(trifluoromethyl)phenyl)acetic acid (23 mg, 0.103 mmol), and was obtained as an off-white solid (17 mg, 30% yield). ¹H NMR (400 MHz, DMSO-d₆) δ=8.37 (s, 1H), 7.90 (d, J 8.4 Hz, 1H), 7.77-7.83 (m, 2H), 7.45-7.48 (m, 2H), 7.25 (t, J 8.4 Hz, 1H), 4.85 (bs, 1H), 4.40 (t, J=8.0 Hz, 2H), 4.19 (s, 2H), 4.11 (s, 2H), 3.31 (t, J=8.0 Hz, 2H), 1.10 (s, 6H); LCMS: 546.2 [M+H].

### Example 24

### 1-(5-(4-AMINO-1-(2-METHOXYETHYL)-1H-PYRAZOLO[3,4-D]PYRIMIDIN-3-YL)-4-FLUOROINDOLIN-1-YL)-2-(2-FLUORO-5-(TRIFLUOROMETHYL)PHENYL)ETHAN-1-ONE (Compound I-24)

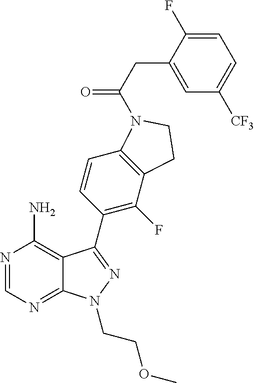 [SMILES: COCCn1nc(-c2ccc3c(c2F)CCN3C(=O)Cc2cc(C(F)(F)F)ccc2F)c2c(N)ncnc21]

The title compound was obtained following the general acid amine coupling procedure, starting from 3-(4-fluoroindolin-5-yl)-1-(2-methoxyethyl)-1H-pyrazolo[3,4-d]pyrimidin-4-amine (D15, 31 mg, 0.094 mmol) and 2-(2-fluoro-(trifluoromethyl)phenyl)acetic acid (21 mg, 0.094 mmol), and was obtained as an off-white solid (12 mg, 24% yield). ¹H NMR (400 MHz, DMSO-d₆) δ=8.23 (s, 1H), 7.93 (d, J 8.4 Hz, 1H), 7.81-7.82 (m, 1H), 7.75-7.76 (m, 1H), 7.47 (t, J 8.8 Hz, 1H), 7.35 (t, J 8.0 Hz, 1H), 4.49 (t, J 5.6 Hz, 2H), 4.41 (t, J 8.4 Hz, 2H), 4.11 (s, 2H), 3.81 (t, J 5.6 Hz, 2H), 3.30-3.32 (m, 2H), 3.22 (s, 3H); LCMS: 533.2 [M+H].

### Example 25

### 1-(5-(4-AMINO-7-CYCLOPROPYL-7H-PYRROLO[2,3-D]PYRIMIDIN-5-YL)-4-FLUOROINDOLIN-1-YL)-2-(4-((4-METHYLPIPERAZIN-1-YL)METHYL)-3-(TRIFLUOROMETHYL)PHENYL)ETHAN-1-ONE (Compound I-25)

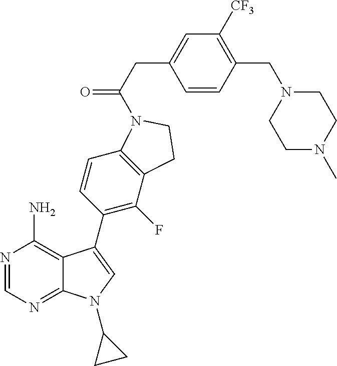 [SMILES: CN1CCN(Cc2ccc(CC(=O)N3CCc4c3ccc(-c3cn(C5CC5)c5ncnc(N)c35)c4F)cc2C(F)(F)F)CC1]

The title compound was obtained following the general acid amine coupling procedure, starting from 7-cyclopropyl-5-(4-fluoroindolin-5-yl)-7H-pyrrolo[2,3-d]pyrimidin-4-amine (D9, 38 mg, 0.123 mmol) and 2-(4-((4-methylpiperazin-1-yl)methyl)-3-(trifluoromethyl)phenyl)acetic acid (E1, 39 mg, 0.123 mmol), and was obtained as pale brown solid (6 mg, 8% yield). ¹H NMR (400 MHz, DMSO-d₆) δ=8.14 (s, 1H), 7.90 (d, J 8.0 Hz, 1H), 7.71 (d, J 8.0 Hz, 1H), 7.63 (s, 1H), 7.55 (d, J 8.0 Hz, 1H), 7.17-7.21 (m, 2H), 6.00 (bs, 2H), 4.32 (t, J 8.4 Hz, 2H), 3.99 (s, 2H), 3.56-3.56 (m, 3H), 3.26-3.28 (m, 2H), 2.32-2.36 (m, 8H), 2.16 (s, 3H), 1.00-1.04 (m, 4H); LCMS: 608.3 [M+H].

### Example 26

### 1-(5-(4-AMINO-7-(2-METHOXYETHYL)-7H-PYRROLO[2,3-D]PYRIMIDIN-5-YL)-4-FLUOROINDOLIN-1-YL)-2-(2-FLUORO-5-(TRIFLUOROMETHYL)PHENYL)ETHAN-1-ONE (Compound I-26)

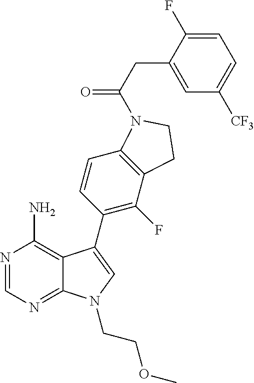 [SMILES: COCCn1cc(-c2ccc3c(c2F)CCN3C(=O)Cc2cc(C(F)(F)F)ccc2F)c2c(N)ncnc21]

The title compound was obtained following the general acid amine coupling procedure, starting from 5-(4-fluoroindolin-5-yl)-7-(2-methoxyethyl)-7H-pyrrolo[2,3-d]pyrimidin-4-amine (D16, 25 mg, 0.076 mmol) and 2-(2-fluoro-5-(trifluoromethyl)phenyl)acetic acid (17 mg, 0.076 mmol), and was obtained as an off-white solid (15 mg, 36% yield). ¹H NMR (400 MHz, DMSO-d₆) δ=8.13 (s, 1H), 7.88 (d, J 8.20 Hz, 1H), 7.81-7.83 (m, 1H), 7.75-7.77 (m, 1H), 7.47 (t, J 8.92 Hz, 1H), 7.29 (s, 1H), 7.20 (t, J 7.96 Hz, 1H), 6.05 (bs, 2H), 4.39 (t, J 8.48 Hz, 2H), 4.33 (t, J 5.24 Hz, 2H), 4.09 (s, 2H), 3.71 (t, J 5.36 Hz, 2H), 3.29 (t, J 8.40 Hz, 2H), 3.22 (s, 3H); LCMS: 532.2 [M+H].

### Example 27

### 1-(5-(4-AMINO-7-(3-HYDROXYCYCLOBUTYL)-7H-PYRROLO[2,3-D]PYRIMIDIN-5-YL)-4-FLUOROINDOLIN-1-YL)-2-(2-FLUORO-5-(TRIFLUOROMETHYL)PHENYL)ETHAN-1-ONE (Compound I-27)

### Step 1: Synthesis of 1-(5-(4-amino-7-(3-(benzyloxy)cyclobutyl)-7H-pyrrolo[2,3-d]pyrimidin-5-yl)-4-fluoroindolin-1-yl)-2-(2-fluoro-5-(trifluoromethyl)phenyl)ethan-1-one

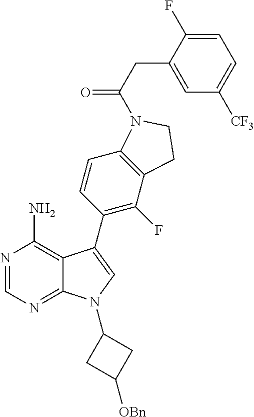 [SMILES: Nc1ncnc2c1c(-c1ccc3c(c1F)CCN3C(=O)Cc1cc(C(F)(F)F)ccc1F)cn2C1CC(OCc2ccccc2)C1]

The title compound was obtained following the general acid amine coupling procedure, starting from 7-(3-(benzyloxy)cyclobutyl)-5-(4-fluoroindolin-5-yl)-7H-pyrrolo[2,3-d]pyrimidin-4-amine (D17, 39 mg, 0.090 mmol) and 2-(2-fluoro-5-(trifluoromethyl)phenyl)acetic acid (20 mg, 0.090 mmol), and was obtained as a brown gum (15 mg, 26% yield). LCMS: 634.2 [M+H].

### Step 2: Synthesis of 1-(5-(4-amino-7-(3-hydroxycyclobutyl)-7H-pyrrolo[2,3-d]pyrimidin-5-yl)-4-fluoroindolin-1-yl)-2-(2-fluoro-5-(trifluoromethyl)phenyl)ethan-1-one

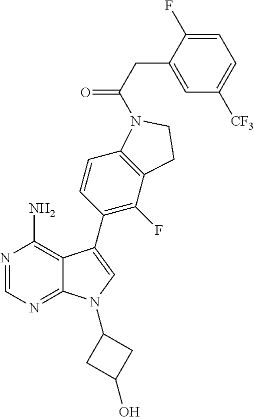 [SMILES: Nc1ncnc2c1c(-c1ccc3c(c1F)CCN3C(=O)Cc1cc(C(F)(F)F)ccc1F)cn2C1CC(O)C1]

Boron trichloride (1M in dichloromethane, 0.126 mL, 0.126 mmol) was added dropwise to a solution of 1-(5-(4-amino-7-(3-(benzyloxy)cyclobutyl)-7H-pyrrolo[2,3-d]pyrimidin-5-yl)-4-fluoroindolin-1-yl)-2-(2-fluoro-5-(trifluoromethyl)phenyl)ethan-1-one (0.010 g, 0.016 mmol) in dry DCM (2 mL) at −60° C. and the resulting solution was stirred at 0° C. for 3 h. Following completion of the reaction (as indicated by TLC and LCMS), the reaction mixture was cooled to −70° C., neutralized with NH₄OH (25% in water), and extracted with DCM (2×2 mL). The combined organic layers were dried over Na₂SO₄, filtered, and concentrated under reduced pressure to give crude material which was purified by preparative HPLC (eluting with 0.1% TFA in water and acetonitrile), affording the title product as a white solid (3 mg, 35% yield). ¹H NMR (400 MHz, CD₃OD) δ=8.33 (s, 1H), 8.02 (d, J 8.4 Hz, 1H), 7.70-7.75 (m, 3H), 7.30-7.38 (m, 2H), 5.59-5.61 (m, 1H), 4.62-4.67 (m, 1H), 4.43-4.48 (m, 2H), 4.09 (s, 2H), 3.33-3.40 (m, 2H), 2.85-2.92 (m, 2H), 2.60-2.64 (m, 2H); LCMS: 544.2 [M+H].

### Example 28

### 1-(5-(4-AMINO-7-(1-METHYLPIPERIDIN-4-YL)-7H-PYRROLO[2,3-D]PYRIMIDIN-5-YL)-4-FLUOROINDOLIN-1-YL)-2-(2-FLUORO-5-(TRIFLUOROMETHYL)PHENYL)ETHAN-1-ONE (Compound I-28)

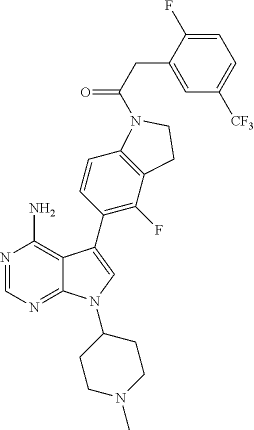 [SMILES: CN1CCC(n2cc(-c3ccc4c(c3F)CCN4C(=O)Cc3cc(C(F)(F)F)ccc3F)c3c(N)ncnc32)CC1]

The title compound was obtained following the general acid amine coupling procedure, starting from 5-(4-fluoroindolin-5-yl)-7-(1-methylpiperidin-4-yl)-7H-pyrrolo[2,3-d]pyrimidin-4-amine (D18, 18 mg, 0.045 mmol) and 2-(2-fluoro-5-(trifluoromethyl)phenyl)acetic acid (10 mg, 0.045 mmol), and was obtained as a white solid (8 mg, 31% yield). ¹H NMR (400 MHz, CD₃OD) δ=8.16 (s, 1H), 7.99 (d, J 8.4 Hz, 1H), 7.69-7.76 (m, 2H), 7.33-7.38 (m, 2H), 7.27 (t, J 8.0 Hz, 1H), 4.59-4.65 (m, 1H), 4.43-4.48 (m, 2H), 4.08 (s, 2H), 3.33 (s, 2H), 3.10-3.16 (m, 2H), 2.37-2.43 (m, 5H), 2.05-2.20 (m, 4H); LCMS: 571.7 [M+H].

### Example 29

### 1-(5-(4-AMINO−1-CYCLOPROPYL-1H-PYRAZOLO[4,3-C]PYRIDIN-3-YL)-4-FLUOROINDOLIN-1-YL)-2-(2-FLUORO-5-(TRIFLUOROMETHYL)PHENYL)ETHAN-1-ONE (Compound I-29)

### Step 1: Synthesis of 1-(5-bromo-4-fluoroindolin-1-yl)-2-(2-fluoro-5-(trifluoromethyl)phenyl)ethan-1-one

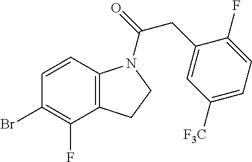 [SMILES: O=C(Cc1cc(C(F)(F)F)ccc1F)N1CCc2c1ccc(Br)c2F]

HATU (3.97 g, 10.44 mmol) and DIPEA (1.657 mL, 9.49 mmol) were added to a solution of 2-(2-fluoro-5-(trifluoromethyl)phenyl)acetic acid (2.108 g, 9.49 mmol) in DMF (5 mL) and the resulting mixture was stirred at 25° C. for 15 min. 5-Bromo-4-fluoroindoline (prepared as reported in PCT Publication No. WO 2021/093795 A1, 2.05 g, 9.49 mmol) was then added and the resulting mixture was stirred at 25° C. for another 12 h. Following completion of the reaction (as indicated by LCMS), the reaction mixture was concentrated under reduced pressure to afford crude material which was purified by flash chromatography (silica gel 230-400 mesh, eluting with 8% EtOAc in petroleum ether), giving the title compound as a pale brown solid. LCMS: 420.1 [M+H].

### Step 2: Synthesis of 1-(4-fluoro-5-(4,4,5,5-tetramethyl-1,3,2-dioxaborolan-2-yl)indolin-1-yl)-2-(2-fluoro-5-(trifluoromethyl)phenyl)ethan-1-one

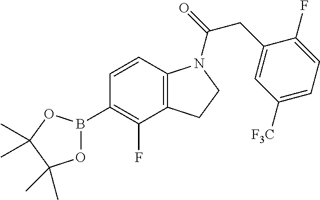 [SMILES: CC1(C)OB(c2ccc3c(c2F)CCN3C(=O)Cc2cc(C(F)(F)F)ccc2F)OC1(C)C]

The title compound was prepared via a similar procedure described for step 4 of intermediate B1, starting from 1-(5-bromo-4-fluoroindolin-1-yl)-2-(2-fluoro-5-(trifluoromethyl)phenyl)ethan-1-one (2.12 g, 5.05 mmol) and using a heating time of 40 h. It was obtained as an off-white solid (0.750 g, 31.8% yield). LCMS: 468.1 [M+H].

### Step 3: Synthesis of 1-(5-(1-cyclopropyl-4-((2,4-dimethoxybenzyl)amino)-1H-pyrazolo[4,3-c]pyridin-3-yl)-4-fluoroindolin-1-yl)-2-(2-fluoro-5-(trifluoromethyl)phenyl)ethan-1-one

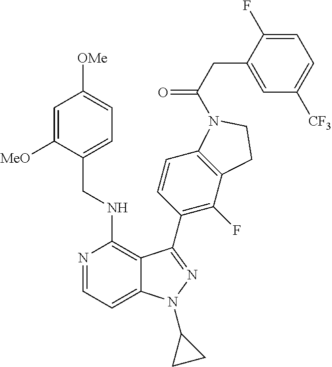 [SMILES: COc1ccc(CNc2nccc3c2c(-c2ccc4c(c2F)CCN4C(=O)Cc2cc(C(F)(F)F)ccc2F)nn3C2CC2)c(OC)c1]

The title compound was obtained following the general Suzuki-Miyaura procedure, starting from 1-cyclopropyl-N-(2,4-dimethoxybenzyl)-3-iodo-1H-pyrazolo[4,3-c]pyridin-4-amine (A15, 0.070 g, 0.155 mmol) and 1-(4-fluoro-5-(4,4,5,5-tetramethyl-1,3,2-dioxaborolan-2-yl)indolin-1-yl)-2-(2-fluoro-5-(trifluoromethyl)phenyl)ethan-1-one (0.073 g, 0.155 mmol), and was obtained as an off-white solid (0.03 g, 29% yield). LCMS: 664.3 [M+H].

### Step 4: Synthesis of 1-(5-(4-amino-1-cyclopropyl-1H-pyrazolo[4,3-c]pyridin-3-yl)-4-fluoroindolin-1-yl)-2-(2-fluoro-5-(trifluoromethyl)phenyl)ethan-1-one

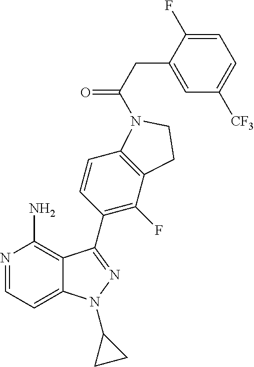 [SMILES: Nc1nccc2c1c(-c1ccc3c(c1F)CCN3C(=O)Cc1cc(C(F)(F)F)ccc1F)nn2C1CC1]

Triethylsilane (5.3 mg, 0.045 mmol) and TFA (5.2 mg, 0.045 mmol) were added to a solution of 1-(5-(1-cyclopropyl-4-((2,4-dimethoxybenzyl)amino)-1H-pyrazolo[4,3-c]pyridin-3-yl)-4-fluoroindolin-1-yl)-2-(2-fluoro-5-(trifluoromethyl)phenyl)ethan-1-one (30 mg, 0.045 mmol) in DCM (2 mL) at 0° C. and the resulting mixture was stirred at 25° C. for 12 h. Following completion of the reaction (as indicated by LCMS), the reaction mixture was concentrated under reduced pressure to give crude material which was purified by preparative HPLC (eluting with 0.1% TFA in water and acetonitrile), affording the title product as an off-white solid (18 mg, 77% yield). ¹H NMR (400 MHz, DMSO-d₆) δ=7.95 (d, J 8.4 Hz, 1H), 7.76-7.83 (m, 3H), 7.47 (t, J 8.8 Hz, 1H), 7.37 (t, J 8.0 Hz, 1H), 7.30 (d, J 7.2 Hz, 1H), 4.43 (t, J 8.4 Hz, 2H), 4.13 (s, 2H), 3.94-3.97 (m, 1H), 3.30 (t, J 8.4 Hz, 2H), 1.18-1.19 (m, 4H); LCMS: 514.2 [M+H].

### Example 30

### 1-(5-(4-AMINO-7-CYCLOPROPYL-7H-PYRROLO[2,3-D]PYRIMIDIN-5-YL)-4-FLUOROINDOLIN-1-YL)-2-(2-((4-METHYLPIPERAZIN-1-YL)METHYL)-5-(TRIFLUOROMETHYL)PHENYL)ETHAN-1-ONE (Compound I-30)

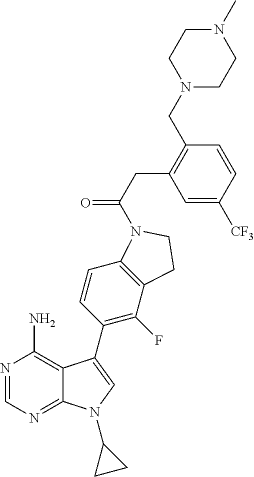 [SMILES: CN1CCN(Cc2ccc(C(F)(F)F)cc2CC(=O)N2CCc3c2ccc(-c2cn(C4CC4)c4ncnc(N)c24)c3F)CC1]

The title compound was obtained following the general acid amine coupling procedure, starting from 7-cyclopropyl-5-(4-fluoroindolin-5-yl)-7H-pyrrolo[2,3-d]pyrimidin-4-amine (D9, 53 mg, 0.171 mmol) and 2-(2-((4-methylpiperazin-1-yl)methyl)-5-(trifluoromethyl)phenyl)acetic acid (E2, 54 mg, 0.171 mmol), and was obtained as an off-white solid (3 mg, 2.9% yield). ¹H NMR (400 MHz, CD₃OD) δ=8.20 (s, 1H), 8.00 (d, J 8.4 Hz, 1H), 7.57-7.60 (m, 2H), 7.47-7.49 (m, 1H), 7.27 (t, J 8.0 Hz, 1H), 7.17 (s, 1H), 4.43-4.49 (m, 2H), 4.07 (s, 2H), 3.66 (s, 2H), 3.51-3.53 (m, 2H), 3.41-3.50 (m, 1H), 2.43-2.70 (m, 8H), 2.09 (s, 3H), 1.13-1.31 (m, 2H), 1.06-1.09 (m, 2H); LCMS: 608.3 [M+H].

### Example 31

### 1-(5-(4-AMINO-7-(1-METHYLPIPERIDIN-3-YL)-7H-PYRROLO[2,3-D]PYRIMIDIN-5-YL)-4-FLUOROINDOLIN-1-YL)-2-(2-FLUORO-5-(TRIFLUOROMETHYL)PHENYL)ETHAN-1-ONE (Compound I-31)

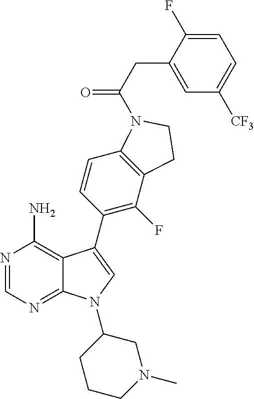 [SMILES: CN1CCCC(n2cc(-c3ccc4c(c3F)CCN4C(=O)Cc3cc(C(F)(F)F)ccc3F)c3c(N)ncnc32)C1]

The title compound was obtained following the general Suzuki-Miyaura coupling procedure, starting from 5-iodo-7-(1-methylpiperidin-3-yl)-7H-pyrrolo[2,3-d]pyrimidin-4-amine (A16, 14 mg, 0.039 mmol) and 1-(4-fluoro-5-(4,4,5,5-tetramethyl-1,3,2-dioxaborolan-2-yl)indolin-1-yl)-2-(2-fluoro-5-(trifluoromethyl)phenyl)ethan-1-one (step 2 of Example 29, 18 mg, 0.039 mmol), and was obtained as an off-white solid (3.9 mg, 18% yield). ¹H NMR (400 MHz, CD₃OD) δ=8.16 (s, 1H), 7.99 (d, J=8.0 Hz, 1H), 7.75 (d, J 6.8 Hz, 1H), 7.69-7.72 (m, 1H), 7.33-7.38 (m, 2H), 7.27 (t, J 8.0 Hz, 1H), 4.44 (t, J 8.4 Hz, 2H), 4.08 (s, 2H), 3.33-3.38 (m, 2H), 3.15-3.19 (m, 1H), 2.95-2.98 (m, 1H), 2.63-2.66 (m, 1H), 2.45 (s, 3H), 2.33 (bs, 1H), 2.11-2.13 (m, 1H), 1.84-1.97 (m, 4H); LCMS: 571.2 [M+H].

### Example 32

### 1-(5-(4-AMINO-7-CYCLOPROPYL-7H-PYRROLO[2,3-D]PYRIMIDIN-5-YL)-4-FLUOROINDOLIN-1-YL)-2-(3-((4-METHYLPIPERAZIN-1-YL)METHYL)-5-(TRIFLUOROMETHYL)PHENYL)ETHAN-1-ONE (Compound I-32)

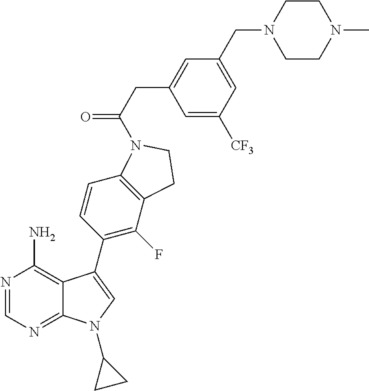 [SMILES: CN1CCN(Cc2cc(CC(=O)N3CCc4c3ccc(-c3cn(C5CC5)c5ncnc(N)c35)c4F)cc(C(F)(F)F)c2)CC1]

The title compound was obtained following the general acid amine coupling procedure, starting from 7-cyclopropyl-5-(4-fluoroindolin-5-yl)-7H-pyrrolo[2,3-d]pyrimidin-4-amine (D9, 30 mg, 0.097 mmol) and 2-(3-((4-methylpiperazin-1-yl)methyl)-5-(trifluoromethyl)phenyl)acetic acid (E3, 31 mg, 0.097 mmol), and was obtained as an off-white solid (7.5 mg, 13% yield). ¹H NMR (400 MHz, DMSO-d₆) δ=8.39 (s, 1H), 7.92 (d, J 8.0 Hz, 1H), 7.60 (bs, 2H), 7.55 (s, 1H), 7.48 (s, 1H), 7.23 (t, J 8.0 Hz, 1H), 4.36 (t, J 8.4 Hz, 2H), 4.05 (s, 2H), 3.66-3.71 (m, 3H), 3.39-3.41 (m, 2H), 3.27 (t, J 8.8 Hz, 2H), 2.90-3.05 (m, 4H), 2.79 (s, 3H), 2.40-2.42 (m, 2H), 1.09-1.10 (m, 4H); LCMS: 608.3 [M+H].

### Example 33

### 1-(5-(4-AMINO-7-(1-METHYLAZETIDIN-3-YL)-7H-PYRROLO[2,3-D]PYRIMIDIN-5-YL)-4-FLUOROINDOLIN-1-YL)-2-(2-FLUORO-5-(TRIFLUOROMETHYL)PHENYL)ETHAN-1-ONE (Compound I-33)

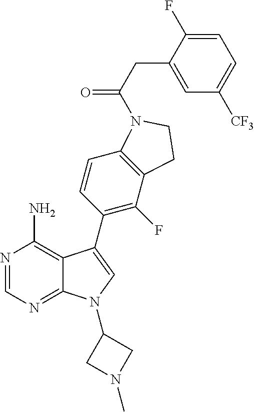 [SMILES: CN1CC(n2cc(-c3ccc4c(c3F)CCN4C(=O)Cc3cc(C(F)(F)F)ccc3F)c3c(N)ncnc32)C1]

The title compound was obtained following the general Suzuki-Miyaura coupling procedure, starting from 5-iodo-7-(1-methylazetidin-3-yl)-7H-pyrrolo[2,3-d]pyrimidin-4-amine (A17, 15 mg, 0.046 mmol) and 1-(4-fluoro-5-(4,4,5,5-tetramethyl-1,3,2-dioxaborolan-2-yl)indolin-1-yl)-2-(2-fluoro-5-(trifluoromethyl)phenyl)ethan-1-one (step 2 of Example 29, 21 mg, 0.046 mmol), and was obtained as an off-white solid (3 mg, 12% yield). ¹H NMR (400 MHz, DMSO-d₆) δ=8.28 (s, 1H), 7.91 (d, J 8.4 Hz, 1H), 7.76-7.82 (m, 3H), 7.47 (t, J 8.8 Hz, 1H), 7.23 (t, J 8.0 Hz, 1H), 6.74 (s, 2H), 5.58-5.62 (m, 1H), 4.81 (bs, 1H), 4.64-4.66 (m, 2H), 4.49 (bs, 1H), 4.40 (t, J 8.4 Hz, 2H), 4.10 (s, 2H), 3.29 (t, J=8.8 Hz, 2H), 2.68 (s, 3H); LCMS: 543.2 [M+H].

### Example 34

### 3-(4-AMINO-5-(4-FLUORO-1-(2-(2-FLUORO-5-(TRIFLUOROMETHYL)PHENYL)ACETYL)INDOLIN-5-YL)-7H-PYRROLO[2,3-D]PYRIMIDIN-7-YL)PROPANOIC ACID (Compound I-34)

### Step 1: Synthesis of tert-butyl 3-(4-amino-5-(4-fluoro-1-(2-(2-fluoro-5-(trifluoromethyl)phenyl)acetyl)indolin-5-yl)-7H-pyrrolo[2,3-d]pyrimidin-7-yl)propanoate

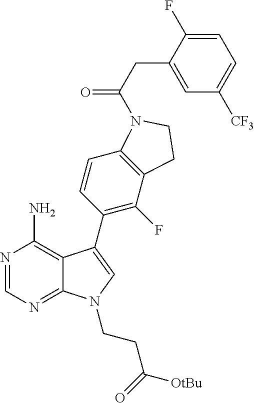 [SMILES: CC(C)(C)OC(=O)CCn1cc(-c2ccc3c(c2F)CCN3C(=O)Cc2cc(C(F)(F)F)ccc2F)c2c(N)ncnc21]

The title compound was obtained following the general Suzuki-Miyaura coupling procedure, starting from tert-butyl 3-(4-amino-5-iodo-7H-pyrrolo[2,3-d]pyrimidin-7-yl)propanoate (A18, 0.05 g, 0.129 mmol) and 1-(4-fluoro-5-(4,4,5,5-tetramethyl-1,3,2-dioxaborolan-2-yl)indolin-1-yl)-2-(2-fluoro-5-(trifluoromethyl)phenyl)ethan-1-one (step 2 of Example 29, 0.06 g, 0.129 mmol), and was obtained as a brown gum (0.02 g, 26% yield). LCMS: 602.2 [M+H].

### Step 2: Synthesis of 3-(4-amino-5-(4-fluoro-1-(2-(2-fluoro-5-(trifluoromethyl)phenyl)acetyl)indolin-5-yl)-7H-pyrrolo[2,3-d]pyrimidin-7-yl)propanoic acid

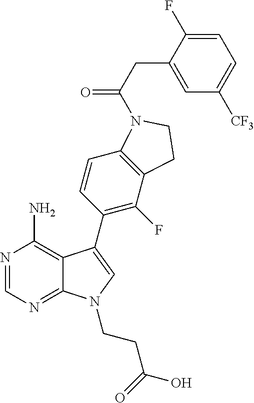 [SMILES: Nc1ncnc2c1c(-c1ccc3c(c1F)CCN3C(=O)Cc1cc(C(F)(F)F)ccc1F)cn2CCC(=O)O]

TFA (0.1 mL) was added to a solution of tert-butyl 3-(4-amino-5-(4-fluoro-1-(2-(2-fluoro-5-(trifluoromethyl)phenyl)acetyl)indolin-5-yl)-7H-pyrrolo[2,3-d]pyrimidin-7-yl)propanoate (20 mg, 0.033 mmol) in DCM (2 mL) at 0° C. and the resulting mixture was stirred at 25° C. for 12 h. Following completion of the reaction (as indicated by LCMS), the reaction mixture was concentrated under reduced pressure to afford crude material which was purified by preparative HPLC (eluting with 0.1% TFA in water and acetonitrile), giving the title product as an off-white solid (6 mg, 33% yield). ¹H NMR (400 MHz, DMSO-d₆) δ=12.48 (bs, 1H), 8.38 (s, 1H), 7.90 (d, J 8.4 Hz, 1H), 7.82 (d, J 6.4 Hz, 1H), 7.76-7.79 (m, 1H), 7.57 (s, 1H), 7.47 (t, J 9.2 Hz, 1H), 7.22 (t, J 8.0 Hz, 1H), 4.38-4.48 (m, 4H), 4.11 (s, 2H), 2.73-2.89 (m, 4H); LCMS: 546.2 [M+H].

### Biological Example 1

### NEK7 Enzymatic Assay

Casein substrate (from bovine milk, hydrolyzed and partially dephosphorylated mixture of α, β and κ caseins, obtained from Sigma Aldrich, catalogue #C4765, diluted in distilled water to a final concentration of 1 mg/mL, assay concentration 20 μM) and full-length recombinant human Nek7 (expressed by baculovirus in Sf9 insect cells using a N-terminal GST tag, obtained from SignalChem, catalogue #N09-10G, 0.1 μg/μL, assay concentration 70 nM) were mixed in assay buffer (20 mM Hepes pH 7.5, 10 mM MgCl₂, 1 mM EGTA, 0.02% Brij35, 0.02 mg/ml BSA, 0.1 mM NB3VO₄, 2 mM DTT, 1% DMSO). Compounds of interest (in DMSO, serial 3-fold dilution from 10 μM to 0.5 nM) or control (1% DMSO) were dispensed into the kinase reaction mixture by Acoustic technology (Echo550; nanoliter range). After incubation at room temperature for 20 minutes, the kinase reaction was initiated by addition of [³²P]-ATP (Specific activity 10 μCi/μl) and the mixture was incubated at room temperature for 2 hours. The reaction was then stopped by spotting the reaction mixture on strips of phosphocellulose P81 paper. Following washing, the radioactivity of the P81 paper was measured and kinase activity data were expressed as the percent remaining kinase activity in test samples compared to vehicle (dimethyl sulfoxide) reactions. IC₅₀ values and curve fits were obtained using Prism (GraphPad Software).

### Biological Example 2

### Biochemical Assays of Representative Compounds

Representative compounds were tested for inhibitory activity against NEK7 according to the procedure described above. Results are given in the table below.

TABLE 2
Activity of Representative compounds of Structure (I)

| | Nek7 |
|---|---|
| Compound | IC₅₀ |
| No. | (nM) |
| I-1 | *** |
| I-2 | **** |
| I-3 | * |
| I-4 | **** |
| I-5 | *** |
| I-6 | * |
| I-7 | **** |
| I-8 | ** |
| I-9 | ** |
| I-10 | **** |
| I-11 | ***** |
| I-12 | ***** |
| I-13 | **** |
| I-14 | ***** |
| I-15 | ***** |
| I-16 | ***** |
| I-17 | ***** |
| I-18 | ***** |
| I-19 | ***** |
| I-20 | ***** |
| I-21 | ***** |
| I-22 | ***** |
| I-23 | ***** |
| I-24 | ***** |
| I-25 | *** |
| I-26 | ***** |
| I-27 | ***** |
| I-28 | ***** |
| I-29 | ***** |
| I-30 | * |
| I-31 | ***** |
| I-32 | * |
| I-33 | ***** |
| I-34 | ***** |

For Nek 7 IC₅₀ activity in Table 2:
* represents a value above 1,500 nM
** represents a value between 501 up to 1,500 nM
*** represents a value between 301 up to 500 nM
**** represents a value between 151 up to 300 nM
***** represents a value below 150
— denotes a value was not determined

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including but not limited to Provisional Application Ser. No. 63/167,523 filed Mar. 29, 2021, and Provisional Application Ser. No. 63/185,274 filed May 6, 2021, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A compound having the following structure (I): 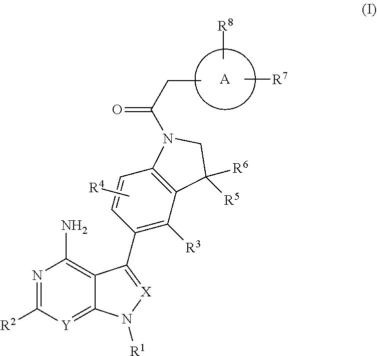
or a pharmaceutically acceptable salt, stereoisomer or prodrug thereof, wherein:
A is C₆-C₁₀ aryl or 5- or 6-membered heteroaryl;
X is N or CR⁹;
Y is N or CH;
R¹ is H, C₂-C₆ alkyl, C₂-C₆ alkenyl, C₂-C₆ alkynyl, C₃-C₈ cycloalkyl, 3-8 membered heterocyclyl, or 5- or 6-membered heteroaryl, each of which is optionally substituted with one more substituents selected from halo, hydroxyl, cyano, aminyl, carboxy, C₁-C₆ alkyl, C₂-C₆ alkenyl, C₂-C₆ alkynyl, C₁-C₆ alkoxy, and 3-8 membered heterocyclyl;
R² is H or C₁-C₆ alkyl;
R³ is halo;
R⁴ is H, halo, or C₁-C₆ alkyl;
R⁵ and R⁶ are each independently H, halo, C₁-C₆ alkyl, or C₁-C₆ haloalkyl, or R⁵ and R⁶, together with the carbon to which they are attached, form a C₃-C₈ cycloalkyl or 3-8 membered heterocyclyl;
R⁷ is C₁-C₆ alkyl, halo, C₁-C₆ haloalkyl, C₁-C₆ haloalkoxy, 3-8 membered heterocyclyl, or 3-8 membered heterocyclylalkyl;
R⁸ is H, halo, C₁-C₆ alkyl, C₁-C₆ haloalkyl, or C₁-C₆ haloalkoxy; and
R⁹ is H, C₁-C₆ alkyl, C₁-C₆ haloalkyl, C₃-C₈ cycloalkyl, or 3-8 membered heterocyclyl.

2. The compound of claim 1, wherein X is CR⁹ and R⁹ is H.

3-4. (canceled)

5. The compound of claim 1, wherein X is N.

6. The compound of claim 1, wherein Y is N.

7. The compound of claim 1, wherein Y is CH.

8. The compound of claim 1, wherein A is phenyl or pyridinyl.

9-15. (canceled)

16. The compound of claim 1, C₂-C₆ alkyl, cyclopropyl or cyclobutyl, piperidinyl, azetidinyl, oxetanyl, or tetrapyranyl.

17. The compound of claim 1, wherein R¹ has one of the following structures:

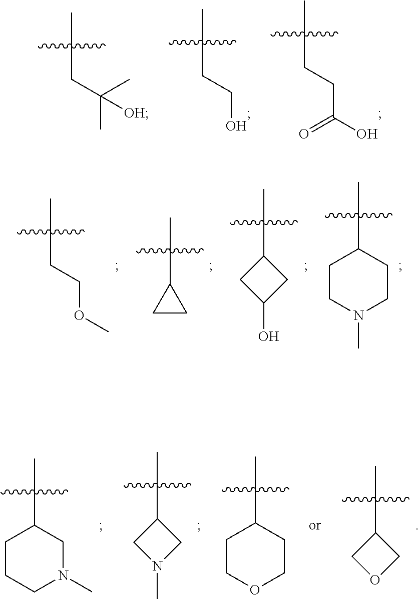

18-21. (canceled)

22. The compound of claim 1, wherein R² is H.

23. (canceled)

24. The compound of claim 1, wherein R³ is fluoro or chloro.

25-26. (canceled)

27. The compound of claim 1, wherein R⁴ is H.

28. (canceled)

29. The compound of claim 1, wherein R⁵ and R⁶ are both H.

30-43. (canceled)

44. The compound of claim 1, wherein R⁷ is methyl, fluoro, trifluoromethyl, trifluoromethoxy, or has the following structure:

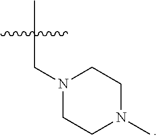 [SMILES: *CN1CCN(C)CC1]

45. The compound of claim 1, wherein R⁸ is H, trifluoromethyl, methyl, or fluoro.

46-52. (canceled)

53. A compound having one of the following structures: 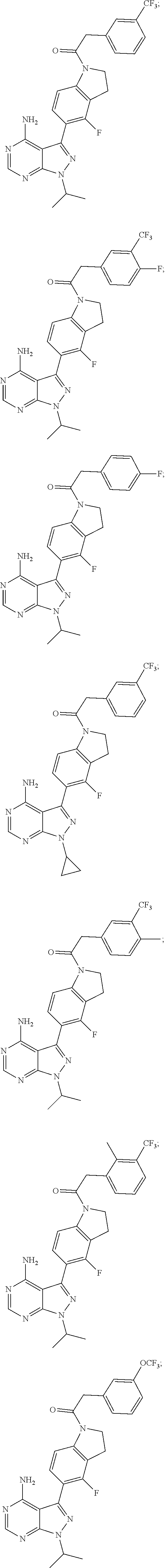 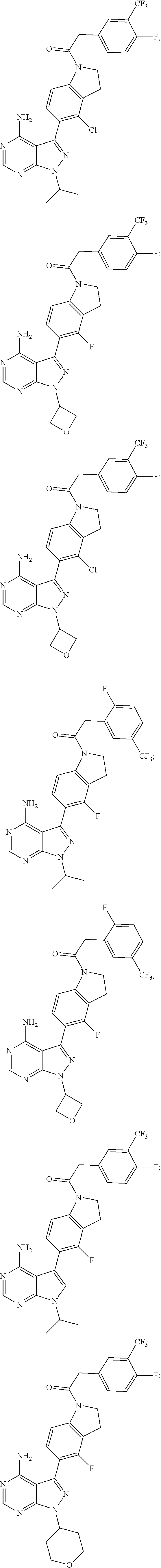 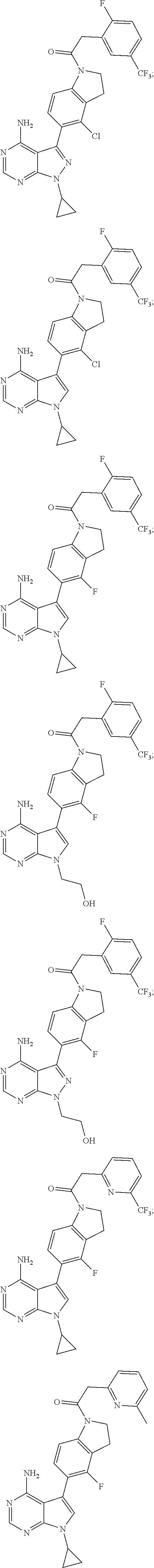 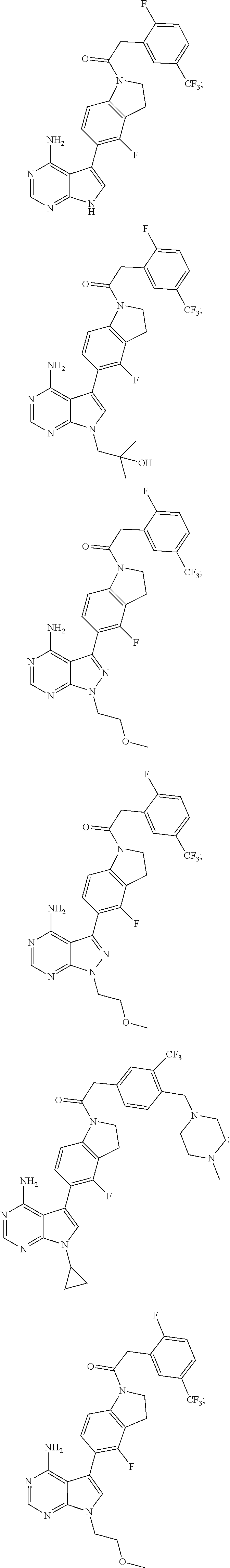 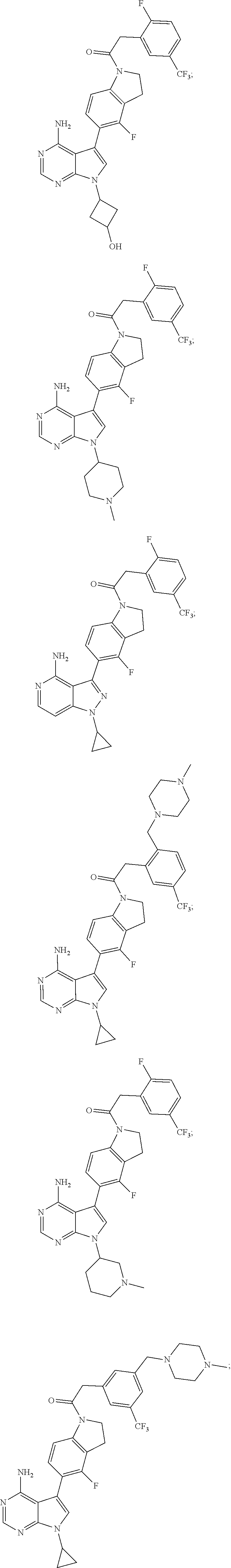 [SMILES: Nc1ncnc2c1c(-c1ccc3c(c1F)CCN3C(=O)Cc1cc(C(F)(F)F)ccc1F)cn2C1CC(O)C1 ; CN1CCC(n2cc(-c3ccc4c(c3F)CCN4C(=O)Cc3cc(C(F)(F)F)ccc3F)c3c(N)ncnc32)CC1 ; Nc1nccc2c1c(-c1ccc3c(c1F)CCN3C(=O)Cc1cc(C(F)(F)F)ccc1F)nn2C1CC1 ; CN1CCN(Cc2ccc(C(F)(F)F)cc2CC(=O)N2CCc3c2ccc(-c2cn(C4CC4)c4ncnc(N)c24)c3F)CC1 ; CN1CCCC(n2cc(-c3ccc4c(c3F)CCN4C(=O)Cc3cc(C(F)(F)F)ccc3F)c3c(N)ncnc32)C1 ; CN1CCN(Cc2cc(CC(=O)N3CCc4c3ccc(-c3cn(C5CC5)c5ncnc(N)c35)c4F)cc(C(F)(F)F)c2)CC1] 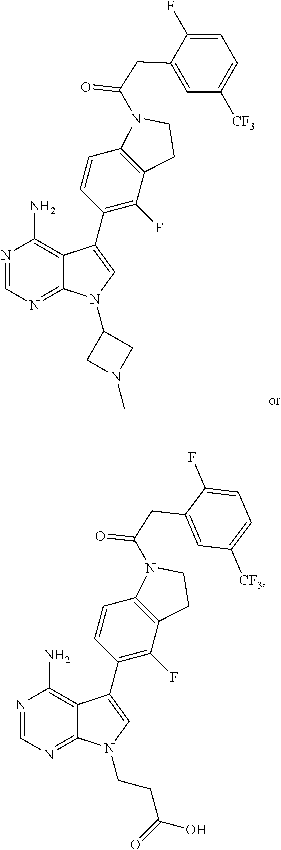 [SMILES: CN1CC(n2cc(-c3ccc4c(c3F)CCN4C(=O)Cc3cc(C(F)(F)F)ccc3F)c3c(N)ncnc32)C1 ; Nc1ncnc2c1c(-c1ccc3c(c1F)CCN3C(=O)Cc1cc(C(F)(F)F)ccc1F)cn2CCC(=O)O]
or a pharmaceutically acceptable salt, stereoisomer, or prodrug thereof.

54. The compound of claim 1, wherein the compound is an inhibitor of NEK7 or a modulator of the NLRP3 inflammasome.

55. (canceled)

56. A pharmaceutical composition comprising the compound of claim 1, and a pharmaceutically acceptable carrier, diluent, or excipient.

57. A method of treating an NLRP3-mediated disease or disorder, comprising administering a therapeutically effective amount of a compound of claim 1, to a subject in need thereof, wherein the disorder is selected from auto-immune, inflammatory disorders, cardiovascular diseases, neurodegenerative disorders, bacterial and viral infections, allergy, asthma, pancreatitis, multi-organ failure, kidney diseases, platelet aggregation, cancer, transplantation, sperm motility, erythrocyte deficiency, graft rejection, lung injuries, respiratory diseases, ischemic conditions, and combinations thereof.

58-59. (canceled)

60. The method of claim 57, wherein the disorder is selected from type II diabetes, atherosclerosis, Alzheimer's disease, aging, fatty liver, metabolic syndrome, asthma, psoriasis, obesity, acute and chronic tissue damage caused by infection, gout, arthritis, macular degeneration, enteritis, hepatitis, peritonitis, silicosis, UV-induced skin sunburn, contact hypersensitivity, sepsis, cancer, neurodegenerative disease, multiple sclerosis, and combinations thereof.

61. The method of claim 57, wherein the disease or disorder is cancer is selected from the group consisting of sarcoma, melanoma, carcinoma, a solid tumor, and combinations thereof.

62. (canceled)
